(19) United States
(12) Patent Application Publication
(10) Pub. No.: US 2024/0350419 A1
(43) Pub. Date: Oct. 24, 2024

(54) ORAL ROOM-TEMPERATURE STABLE YEAST PARTICLES (YP) AND METHODS OF USE

(71) Applicant: UNIVERSITY OF MASSACHUSETTS, Boston, MA (US)

(72) Inventors: Gary R. Ostroff, Worcester, MA (US); Ernesto Soto, Worcester, MA (US)

(21) Appl. No.: 18/611,933

(22) Filed: Mar. 21, 2024

Related U.S. Application Data
(60) Provisional application No. 63/453,800, filed on Mar. 22, 2023.

(51) Int. Cl. A61K 9/50; A61K 39/00; A61K 47/69; A61P 37/04

(52) U.S. Cl. CPC A61K 9/5068; A61K 9/5026; A61K 9/5036; A61K 39/0002; A61K 47/6901; A61P 37/04; A61K 2039/55588

(57) ABSTRACT

The present disclosure provides a gastric resistant, enteric coated nano-silica yeast particle (YP) delivery system. The disclosure further provides methods of making and methods of using a nano-silica yeast particle delivery system.

### RELATED APPLICATIONS

The present invention claims the benefit of U.S. Provisional Application No. 63/453,800, filed Mar. 22, 2023, the contents of which are incorporated herein by reference in its entirety for all purposes.

### STATEMENT OF FEDERALLY SPONSORED RESEARCH

This invention was made with government support under Grant No. R25 HL092610-11 awarded by the National Institutes of Health. The government has certain rights in the invention.

### BACKGROUND

Drug delivery systems are designed to provide a biocompatible reservoir of an active agent for the controlled release of the active agent dependent either on time, or on local conditions, such as pH. There has been continuing interest in microscopic drug delivery systems such as microcapsules, microparticles and liposomes.

Yeast particles (YPs) are hollow, spherical particles about 2-4 μm in diameter that can be used for delivery of a drug payload. Due to their beta-glucan content, yeast particles can be targeted to phagocytic cells, such as macrophages and cells of lymphoid tissue.

Previous efforts to encapsulate payloads inside YPs include loading of soluble payload and trapping polymer components through the glucan hydrocolloid shell, and reacting them to form insoluble complexes trapped inside the shells, or through the layer by layer (LbL) absorption of a soluble payload component(s) onto the surface of a pre-existing YP encapsulated polyplexes or preformed nanoparticles. These trapping methods offer the advantages of very efficient payload trapping, phagocytic cell-targeted uptake of the YPs, and payload release in macrophages and dendritic cells. YP encapsulating nanocomplexes composed of serum albumin-yeast RNA (yRNA), inorganic crystalline matrices such as insoluble calcium, alum hydrocolloid nanoplexes (chitosan, calcium alginate) are amongst vaccine formulation methods that have been published as effective and biocompatible, and resulting in strong, protective immune responses in animal models via systemic administration. However, these current YP drug delivery formulations still have limitations as these methods do not provide for room temperature thermal stability to eliminate the formulation cold chain storage process, and do not offer significant protection against acid and hydrolase degradation following oral delivery through the stomach and small intestine. Furthermore, there is limited uptake of current YP drug delivery formulations into the intestinal epithelial cells.

Thus, there is a need in the pharmaceutical arts for an oral delivery system for targeted delivery of payloads to the intestine while preventing enzymatic and thermal degradation in the upper digestive tract.

### SUMMARY

In one aspect a gastric resistant, enteric coated nano-silica yeast particle (YP) delivery system is provided comprising (i) a YP with a hollow inner cavity and at least one first payload, wherein the at least one first payload is substantially encapsulated by a nano-silica cage, and wherein the nano-silica cage and the at least one first payload are both confined within the hollow inner cavity of the YP; (ii) the YP surface is modified with a mucus penetrating layer, optionally a polydopamine (PDA) layer; (iii) the YP surface is further modified to display a receptor ligand; and (iv) The YP is coated with an enteric coat.

In another aspect a gastric resistant, enteric coated nano-silica yeast particle (YP) delivery system is provided comprising (i) a YP with a hollow inner cavity and at least one first payload, wherein the at least one first payload is completely encapsulated by a nano-silica cage, and wherein the nano-silica cage and the at least one first payload are both confined within the hollow inner cavity of the YP; (ii) the YP surface is modified with a mucus penetrating layer, optionally a polydopamine (PDA) layer; (iii) the YP surface is further modified to display a receptor ligand; and (iv) the YP is coated with an enteric coat.

In certain exemplary embodiments the, YP is selected from the group consisting of a yeast cell wall particle (YCWP), a glucan particle (GP), a yeast glucan particle (YGP), a yeast glucan-mannan particle (YGMP), a glucan lipid particle (GLP), a whole glucan particle (WGP), a glucan mannan lipid particle (GMLP), a glucan chitin particle (GCP) and a mixture thereof.

In certain exemplary embodiments, the at least one first payload is selected from the group consisting of a protein, a peptide, a peptide antigen, an enzyme, an antibody, a nanobody, an antigen binding fragment of an antibody, a single stranded nucleic acid, a double stranded nucleic acid, and a mixture thereof.

In certain exemplary embodiments, the nano-silica cage comprises a chemical selected from the group consisting of tetraethylorthosilicate (TEOS), tetraethylorthogermanate (TEOG), tetramethylorthosilicate (TMOS), aminopropyl triethoxysilicate (APTES), 3-(triethoxysylil)-propyl-isocyanate (TEPI), bis[3-triethoxysilyl) propyl]disulfide (BTEPDS), bis[3-(triethoxysilyl) propyl]tetrasulfide (BTEPTS) and a combination thereof.

In certain exemplary embodiments, the nano-silica cage comprises polymerized tetraethylorthosilicate (TEOS).

In certain exemplary embodiments, the nano-silica cage comprises polymerized tetraethylorthogermanate (TEOG).

In certain exemplary embodiments, the gastric resistant, enteric coated nano-silica yeast particle (YP) delivery system further comprises a coating polymer in the hollow inner cavity, wherein the coating polymer is located on the outside of the nano-silica cage, or on the outside of the YP, wherein the coating polymer is nontoxic and has no pharmacologic activity.

In certain exemplary embodiments, the coating polymer resists breakdown in the presence of gastric fluids in the oral cavity, esophagus, or stomach.

In certain exemplary embodiments, the coating polymer disintegrates in the small intestine.

In certain exemplary embodiments, the coating polymer is chosen from the group consisting of methacrylic acid methylmethacrylate copolymer, methacrylic acid ethyl acrylate copolymer, cellulose acetate phthalate (CAP), cellulose acetate trimellate (CAT), hydroxy propyl methyl cellulose phthalate (HPMCP), hydroxyl propyl methyl cellulose acetate succinate (HPMCAS), polyvinyl acetate (PVAP), methacrylic acid polymer, and any combination thereof.

In certain exemplary embodiments, the payload is stable after a short-term or a long-term exposure to high temperature.

In certain exemplary embodiments, the payload is stable after exposure to a temperature of 25° C., 45° C., or 95° C.

In certain exemplary embodiments, the payload is stable after exposure to the said high temperature for about 30 minutes, about 2 hours, about 5 hours, 15 days, 30 days, 45 days, 60 days, 75 days or 90 days.

In certain exemplary embodiments, the gastric resistant, enteric coated nano-silica yeast particle (YP) delivery system further comprises a second payload, wherein the second payload is not confined in the nano-silica cage.

In certain exemplary embodiments, the second payload is selected from the group consisting of a protein, a peptide, a peptide antigen, an enzyme, an antibody, an antigen binding fragment of an antibody, a single stranded nucleic acid, a double stranded nucleic acid, and a mixture thereof.

In certain exemplary embodiments, the receptor ligand is an intestinal epithelial cell surface receptor ligand.

In certain exemplary embodiments, the receptor ligand is an intestinal epithelial cell surface receptor ligand selected from a group consisting of gamma-polyglutamic acid (γ-PGA), wheat germ agglutinin (WGA), concanavalin-A, (ConA), C-terminal Src kinase (CSK) peptide, arginylglycylaspartic acid (RGD) peptide, Fc fragment and a combination thereof.

In certain exemplary embodiments, the enteric coat is a polymer.

In certain exemplary embodiments, the enteric coat is a polymer selected from a group consisting of cellulose acetate phthalate (CAP), cellulose acetate trimellate (CAT), hydroxy propyl methyl cellulose phthalate (HPMCP), hydroxyl propyl methyl cellulose acetate succinate (HPMCAS), polyvinyl acetate (PVAP), and methacrylic acid polymers.

In certain exemplary embodiments the gastric resistant, enteric coated nano-silica yeast particle (YP) delivery system further comprises a pharmaceutically acceptable carrier or excipient.

In another aspect a kit is provided comprising gastric resistant, enteric coated nano-silica yeast particle (YP) delivery system described in any one of the embodiments described above and optional instructions for use.

In another aspect, a method of preparing a gastric resistant, enteric coated nano-silica yeast particle (YP) delivery system is provided comprising the steps of: (a) loading a YP comprising a hollow inner cavity with at least one first payload; and (b) resuspending the YP in partially pre-polymerized tetrahydroorthosilicate (TEOS) in half hydrodynamic volume, wherein the partially pre-polymerized TEOS is partially pre-polymerized at a pH of about 2 to about 4, wherein the TEOS polymerizes to form a nano-silica cage within the hollow inner cavity, and wherein the nano-silica cage substantially encapsulates the at least one first payload at an encapsulation efficiency of at least 90%, and wherein the nano-silica cage and the at least one first payload are both confined within the hollow inner cavity of the YP, (c) coating the surface of the YP with a mucus penetrating layer, optionally a polydopamine (PDA) layer; (d) modifying the surface of the YP to display a receptor ligand.

In another aspect a method of preparing a gastric resistant, enteric coated nano-silica yeast particle (YP) delivery system comprising the steps of: (a) loading a YP comprising a hollow inner cavity with at least one first payload; and (b) resuspending the YP in partially pre-polymerized tetrahydroorthosilicate (TEOS) in half hydrodynamic volume, wherein the partially pre-polymerized TEOS is partially pre-polymerized at a pH of about 2 to about 4, wherein the TEOS polymerizes to form a nano-silica cage within the hollow inner cavity, and wherein the nano-silica cage substantially encapsulates the at least one first payload at an encapsulation efficiency of at least 90%, and wherein the nano-silica cage and the at least one first payload are both confined within the hollow inner cavity of the YP, (c) coating the surface of the YP with a mucus penetrating layer, optionally a polydopamine (PDA) layer; (d) modifying the surface of the YP to display a receptor ligand.

In certain exemplary embodiments of the method, the at least one first payload is selected from the group consisting of a protein, a peptide, a peptide antigen, an enzyme, an antibody, a nanobody, an antigen binding fragment of an antibody, a single stranded nucleic acid, a double stranded nucleic acid, and a mixture thereof.

In certain exemplary embodiments, the method further comprises the step of loading a coating polymer in the YP.

In certain exemplary embodiments, the method further comprises the step of loading a second payload in the YP.

In certain exemplary embodiments, the second payload is selected from the group consisting of a protein, a peptide, a peptide antigen, an enzyme, an antibody, an antigen binding fragment of an antibody, a single stranded nucleic acid, a double stranded nucleic acid, and a mixture thereof.

In certain exemplary embodiments, the receptor ligand is an intestinal epithelial cell surface receptor ligand.

In certain exemplary embodiments, the receptor ligand is an intestinal epithelial cell surface receptor ligand selected from a group consisting of gamma-polyglutamic acid (γ-PGA), wheat germ agglutinin (WGA), concanavalin-A, (ConA), CSKSSDYQC-6-aminohexanoic (Ahx) peptide (CSK) peptide, arginylglycylaspartic acid-6 aminohexanoic (Ahx) peptide (RGD), Fc fragment and a combination thereof.

In certain exemplary embodiments, the enteric coat is a polymer.

In certain exemplary embodiments, the enteric coat is a polymer selected from a group consisting of cellulose acetate phthalate (CAP), cellulose acetate trimellate (CAT), hydroxy propyl methyl cellulose phthalate (HPMCP), hydroxyl propyl methyl cellulose acetate succinate (HPMCAS), polyvinyl acetate (PVAP), and methacrylic acid polymers.

In another aspect, a pharmaceutical composition is provided comprising a gastric resistant, enteric coated nano-silica yeast particle (YP) delivery system comprising: (i) a YP with a hollow inner cavity and at least one first payload, wherein the at least one first payload is substantially encapsulated by a nano-silica cage, and wherein the nano-silica cage and the at least one first payload are both confined within the hollow inner cavity of the YP; (ii) the YP surface is modified with a mucus penetrating layer, optionally a polydopamine (PDA) layer; (iii) the YP surface is further modified to display a receptor ligand; and (iv) The YP is coated with an enteric coat.

In another aspect a pharmaceutical composition is provided comprising a gastric resistant, enteric coated nano-silica yeast particle (YP) delivery system comprising: (i) a YP with a hollow inner cavity and at least one first payload, wherein the at least one first payload is completely encapsulated by a nano-silica cage, and wherein the nano-silica cage and the at least one first payload are both confined within the hollow inner cavity of the YP; (ii) the YP surface is modified with a mucus penetrating layer, optionally a polydopamine (PDA) layer; (iii) the YP surface is further modified to display a receptor ligand; and (iv) The YP is coated with an enteric coat.

In certain exemplary embodiments, the pharmaceutical composition comprises at least one first payload is selected from the group consisting of a protein, a peptide, a nucleic acid, and any combination thereof.

In another aspect, a method of treating a disease condition in a subject is provided, comprising administering the pharmaceutical composition of any one of the previous embodiments to a subject in need thereof.

In another aspect a vaccine is provided comprising a gastric resistant, enteric coated nano-silica yeast particle (YP) delivery system comprising: (i) a YP with a hollow inner cavity and at least one first payload, wherein the at least one first payload is substantially encapsulated by a nano-silica cage, and wherein the nano-silica cage and the at least one first payload are both confined within the hollow inner cavity of the YP; (ii) the YP surface is modified with a mucus penetrating layer, optionally a polydopamine (PDA) layer; (iii) the YP surface is further modified to display a receptor ligand; and (iv) The YP is coated with an enteric coat.

In another aspect, a vaccine is provided comprising a gastric resistant, enteric coated nano-silica yeast particle (YP) delivery system comprising: (i) a YP with a hollow inner cavity and at least one first payload, wherein the at least one first payload is completely encapsulated by a nano-silica cage, and wherein the nano-silica cage and the at least one first payload are both confined within the hollow inner cavity of the YP; (ii) the YP surface is modified with a mucus penetrating layer, optionally a polydopamine (PDA) layer; (iii) the YP surface is further modified to display a receptor ligand; and (iv) The YP is coated with an enteric coat.

In certain exemplary embodiments, the at least one first payload is selected from the group consisting of a protein, a peptide, a glycoprotein, a lipoprotein, a toxoid, a polysaccharide, a nucleic acid and any combination thereof.

In another aspect a method of preventing a disease condition in a subject is provided, comprising administering to the subject a vaccine of any one of the previous embodiments.

### BRIEF DESCRIPTION OF THE DRAWINGS

The foregoing and other features and advantages of the present disclosure will be more fully understood from the following detailed description of illustrative embodiments taken in conjunction with the accompanying drawings. This patent or application file contains at least one drawing executed in color. Copies of this patent or patent application publication with color drawing(s) will be provided by the Office upon request and payment of the necessary fee.

FIG. 1 is a schematic diagram showing ensilication of payloads within hollow glucan particles (GPs).

FIG. 2 shows the ensilication efficiency of fluorescently labeled Cy3-siDNA under different conditions. Sample 1: pH 2, HCl, 1 hour at room temperature; sample 2: pH 2, HCl, 1 hour at room temperature, GP-siRNA buffered at pH 7.

FIGS. 3A-3G show the various steps of the procedure for ensilication of payloads within GPs. Initial TEOS (top phase, d=0.93 g/mL) and aqueous (acid or base) solution (bottom phase) (FIG. 3A) when mixed forms a TEOS-aqueous emulsion composed of partially pre-polymerized TEOS far from gel point (FIG. 3B). Upon further incubation TEOS-aqueous solution forms partially pre-polymerized TEOS close to gel point (FIG. 3C) which upon further incubation forms silica gel after full TEOS polymerization (FIG. 3D). When TEOS-aqueous solution close to gel point (FIG. 3C) is loaded (FIG. 3E) into GPs containing payloads (FIG. 3F), full polymerization of TEOS inside GPs leads to ensilication of payloads within GPs (FIG. 3G).

FIGS. 4A-FIG. 4B show efficiency of TEOS partially pre-polymerized at pH 2 to ensilicate rhodamine labeled bovine serum albumin (rBSA) within GPs. FIG. 4A shows a bar graph of percent of rBSA trapped in GPs using various TEOS:rBSA ratios compared conventional methods of trapping payloads using yRNA or Ca₃ (PO₄)₂. FIG. 4B shows brightfield and fluorescent microscopy overlays of fGP rBSA [SiO₂]ₙ formulation (at 12.5 w:w TEOS:rBSA).

FIG. 5 shows brightfield and fluorescent microscopy overlays of GPs with or without rBSA and fluorescent silica shell (TEOS/f-APTES).

FIGS. 6A-6B show the phagocytosis of fGP-BSA samples by macrophages. BSA was encapsulated within GPs via ensilication (FIG. 6A) or conventional trapping methods using yRNA (FIG. 6B).

FIG. 7 shows the cytotoxicity of fGP BSA [SiO₂]ₙ to B6 macrophage cell line.

FIG. 8 are fluorescent micrographs showing the intracellular release of rhodamine labeled BSA ensilicated in GPs or trapped within GPs using yRNA at 3 hours and 24 hours after uptake of GPs by macrophages.

FIG. 9 shows the effect of various pH and pre-polymerization times on TEOS ensilication efficiency. The W:W ratio of TEOS:BSA was 12.5:1. TEOS was partially pre-polymerized for 1, 6, or 20 hours prior to adding mixture to GP-BSA.

FIG. 10 shows the ensilication efficiency using TEOS partially pre-polymerized at pH 2 or pH 7 and GP-BSA prepared in water or phosphate buffered saline (PBS) at pH 7. The details of experimental conditions A, B, C, D are listed in Table 1.

FIG. 11 shows the ensilication efficiency after loading of BSA in GPs in the presence or absence of 6 M urea.

FIG. 12 shows the ensilication efficiency after loading of BSA in GPs in the presence or absence of high concentrations of sodium chloride (NaCl).

FIG. 13 shows the effect of TEOS pre-polymerization in strong or weak acids and TEOS-GP-BSA incubation conditions on ensilication efficiency.

FIG. 14A-FIG. 14C show the schematic of ensilication of first payload followed by encapsulation of a second payload within GPs (FIG. 14A), encapsulation efficiency of ensilicated BSA and chitosan within GPs (FIG. 14B), and brightfield and fluorescent micrographs showing location of rBSA and f-chitosan within GPs (FIG. 14C).

FIGS. 15A-15B depict efficient ensilication of tetraethylorthogermanate (TEOG) at pH 2 (FIG. 15A) and pH 10 (FIG. 15B).

FIG. 16 shows the percent of alginate bound to GP with only TEOS and GPs with cationic alginate or anionic poly-lysine polymers partially ensilicated with TEOS.

FIG. 17 shows the percent of chitosan bound to GPs with only TEOS and GPs with cationic alginate or anionic poly-lysine polymers partially ensilicated with TEOS.

FIGS. 18A-18C schematically depict the procedure to measure BSA after exposure of GP-BSA-[SiO₂]ₙ to simulated gastric fluid (SGF)/pepsin or simulated intestinal fluid (SIF)/pancreatin (FIG. 18A) and graphically depict the percent BSA recovered after exposure to PBS (control) or SGF/pepsin (FIG. 18B) or SIF/pancreatin (FIG. 18C).

FIG. 19 shows the schematic of the procedure for preparation of Eudragit® coated ensilicated payload within GPs.

FIGS. 20A-20B show a flowchart summarizing the steps for extraction and quantification of BSA after incubation of GP containing Eudragit coated ensilicated BSA with SGF/pepsin followed by SIF/pancreatin (FIG. 20A) and a graph showing the percent of BSA remaining in GPs after enzymatic digestion (FIG. 20B).

FIGS. 21A-21E show BSA retention in GPs after prolonged exposure of GPs containing ensilicated BSA to high temperature. Recovered rhodamine labeled BSA quantified by fluorescence (FIG. 21A), recovered BSA quantified by SDS-PAGE densitometry (FIG. 21B), recovered protein on SDS-PAGE (FIG. 21C), and recovered BSA on a native gel (FIG. 21D) are shown along with the flowchart summarizing steps of the experiment (FIG. 21E).

FIGS. 22A-22F show the various steps of the production of silicate capped yeast particles (GPS). Initial TEOS (top phase, d=0.93 g/mL) and aqueous ammonium hydroxide (NH₄OH) solution (bottom phase) (FIG. 22A) when mixed forms a TEOS-NH₄OH aqueous emulsion composed of partially pre-polymerized TEOS far from gel point (FIG. 22B). TEOS-NH₄OH aqueous solution forms partially pre-polymerized TEOS close to gel point (FIG. 22C) which rapidly fully polymerizes to form silica gel (FIG. 22D). When partially pre-polymerized TEOS close to gel point (FIG. 22C) is incubated with GPs containing payloads (FIG. 22E), very rapid (<10 minutes) polymerization of TEOS in basic NH₄OH prevents TEOS from diffusing inside GPs and instead results in polymerization of TEOS on the GP surface resulting in plug sealed or silicate capped YPs (FIG. 22F).

FIGS. 23A-23B show the percent of BSA trapped in GPs in absence of TEOS (control), in the presence of TEOS (ensilication approach), and in the presence of TEOS and ammonium hydroxide (NH₄OH; plug seal approach) (FIG. 23A). (FIG. 23B) shows the fluorescent micrographs of GPs containing ensilicated BSA and plug sealed GP-BSA.

FIG. 24 shows percent of BSA trapped in GPs via ensilication in the presence of 0, 0.1%, 1%, and 10% ammonium hydroxide (NH₄OH) as a polymerization catalyst.

FIG. 25 shows anti chicken ovalbumin (OVA) IgG antibody titers in unvaccinated mice (control) and mice immunized with GP-OVA in which OVA was encapsulated in GPs either by trapping with yRNA or by ensilication.

FIG. 26 shows the survival rate after exposure to a lethal cryptococcal infection of unvaccinated mice and mice vaccinated by standard GP Cpd1 MSA/yRNA and GP Cpd1 TEOS vaccines

FIGS. 27A-27B show the survival rate after exposure to a lethal cryptococcal infection of unvaccinated mice (control) and mice vaccinated with GP with Cda trapped using yRNA and GPs with Cda2 trapped by ensilication (FIG. 27A) and the Cryptococcus colony forming units (CFU) in mice at day 70 post infection (FIG. 27B).

FIG. 28 shows the efficiency of ensilication of lysozyme within GPs at various TEOS:lysozyme weight ratios.

FIGS. 29A-29C schematically depict the procedure to measure lysozyme (FIG. 29A), and shows percent of lysozyme remaining in dry (FIG. 29B) or 0.9% saline (FIG. 29C) GP-lysozyme samples after prolonged exposure to room or high temperature. Free lysozyme was used as control.

FIG. 30 shows the percent lysozyme activity retained after exposure to high temperature in samples containing free lysozyme or GP with ensilicated lysozyme (either dry/lyophilized sample or sample suspended in 0.9% saline).

FIGS. 31A-31B show the percent lysozyme activity retained by free lysozyme, lysozyme encapsulated within GPs, lysozyme trapped in GPs with yRNA, and lysozyme ensilicated within GPs during short-term incubation at 45° C. Samples were stored either as dry, lyophilized powders (FIG. 31A) or liquid samples stored in PBS with 2 mM sodium azide (NaN₃) (FIG. 31B).

FIG. 32 shows the percent lysozyme activity retained by free lysozyme and lysozyme ensilicated within GPs during long-term incubation at 45° C.

FIGS. 33A-33B show the release kinetics of GP-IgG-(SiO2)n IgG in B6 cell line. The GP-IgG-(SiO₂)ₙ diffused throughout the cell cytoplasm after phagocytosis (FIG. 33A) and was detected in intact form for 5 hours (FIG. 33B).

FIGS. 34A-34C show the efficiency with which hairpin peptide was ensilicated within GPs or GMLPs using various TEOS:peptide weight ratios (FIG. 34A); fluorescent micrograph of GMLP-Peptide-SiO2 prepared with TEOS:peptide ratio of 31.1 (FIG. 34B); and percent peptide extracted from GPs using HCl or NaF (FIG. 34C).

FIG. 35 shows the SDS-PAGE of GP pellet and supernatant after ensilication.

FIGS. 36A-36B show the encapsulation efficiency yRNA, dsRNA, siRNA ensilicated within GPs using 90% TEOS and 10% 3-aminopropylthriethoxysilane (APTES) (FIG. 36A) and the molecular structure of TEOS and APTES (FIG. 36B).

FIGS. 37A-37D show the ensilication efficiency of the 300-mer Cy3 RNA within GPs using HCl at pH2 and pH 4 (FIG. 37A); location of RNA within GPs (FIG. 37B); extraction efficiency of RNA from GPs with NaF (FIG. 37C); RNA extracted from GPs (FIG. 37D).

FIG. 38 shows the ensilication efficiency of fluorescently labeled ssDNA under different conditions.

FIGS. 39A-39B show a schematic representation of polydopamine (PDA) coating on glucan particles (GPS) followed by surface derivatization/modification with receptor ligands (FIG. 39A) and the enhanced uptake of ligand-derivatized PDA-YPs in the intestine (FIG. 39B).

FIGS. 40A-40C depict a schematic for payload loading in surface derivatized PDA-coated fluorescent GPs (PDA-fGPs) (FIG. 40A), a bar graph of percent of ovalbumin trapped in fGPs, PDA-fGPs, and surface derivatized PDA-fGPs (FIG. 40B), and fluorescent microscopy images of Cy3 labeled ovalbumin payload encapsulated within fGPs, PDA-fGPs, and PDA-fGPs surface derivatized [ligands used γ-PGA: A-gamma-polyglutamic acid; WGA: wheat germ agglutinin; and ConA-concanavalin-A] (FIG. 40C).

FIGS. 41A-41C show schematic depiction of the intestinal loop model for ex vivo evaluation of GP uptake (FIG. 41A), bar graph showing number of GPs per area of intestine observed in the ex vivo (FIG. 41B), and fluorescent microscopy images control and GPs carrying surface ligands [ligands used: γ-PGA: gamma-polyglutamic acid; WGA —wheat germ agglutinin; and ConA: concanavalin-A] taken up by intestinal cells (FIG. 41C).

FIGS. 42A-42B show the uptake of fGP, PDA-fGPs or ligand-PDA-fGPs [ligands used: γ-PGA: gamma-polyglutamic acid; WGA-wheat germ agglutinin; and ConA: concanavalin-A] by three different cell lines. FIG. 42A shows fluorescent microscopy images of cell lines and FIG. 42B shows bar graphs with percent uptake of GPs.

FIG. 43 shows the biodistribution of fGP, PDA-fGPs or ligand-PDA-fGPs [ligands used: γ-PGA-gamma-polyglutamic acid; RGD-arginylglycylaspartic acid-6 aminohexanoic (Ahx) peptide; and CSK:CSKSSDYQC-Ahx peptide] in the duodenum, jejunum, cecum, colon and spleen of mice after intestinal uptake.

FIG. 44 shows the migration of fGP, PDA-fGPs or ligand-PDA-fGPs [ligands used: Y-PGA-gamma-polyglutamic acid; RGD-arginylglycylaspartic acid-6 aminohexanoic (Ahx) peptide; and CSK:CSKSSDYQC-Ahx peptide] into the underlying gut-associated lymphoid tissue (GALT) of the mouse small intestine after intestinal uptake.

FIGS. 45A-45B show a schematic representation for coating of glucan particles (GPs) with polydopamine (PDA) followed by surface derivatization/modification with receptor ligands (FIG. 45A) and coating of surface derivatized payload carrying GPs with an enteric coat (FIG. 45B) for enhanced resistance to degradation in the digestive tract.

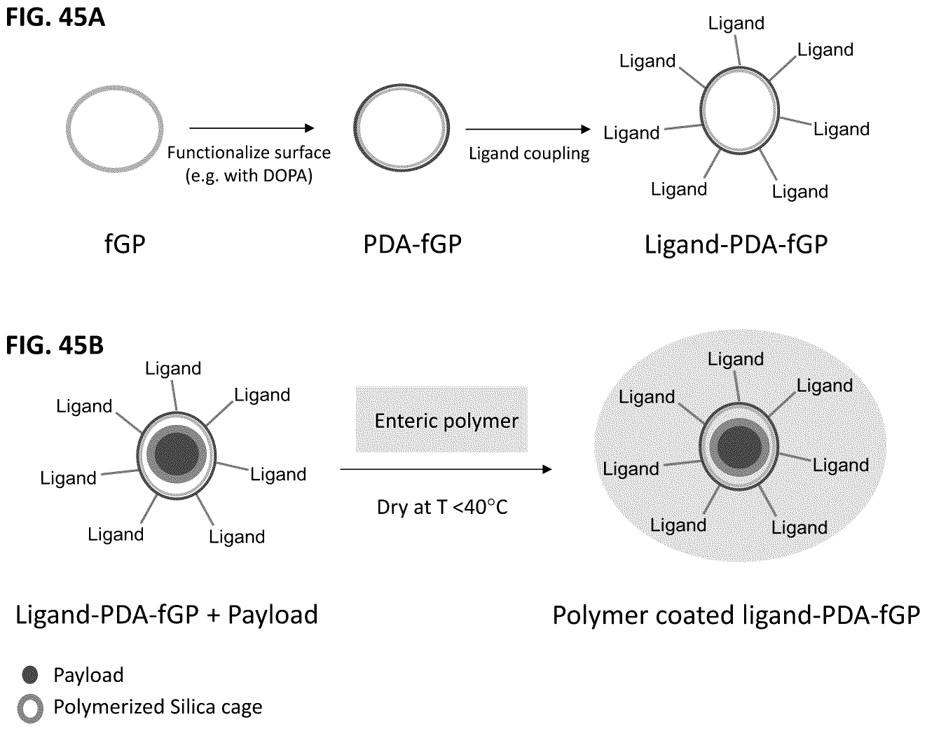

FIGS. 46A-46B show in tabular format the molecular weights, isoelectric points of lysozyme (Lys), ovalbumin (OVA), transferrin (TFN), and glucose oxidase (GOx) and their percent encapsulation efficiency when loaded into GPs with ensilication using 100% TEOS quantified by SDS-PAGE (FIG. 46B) along with the image of the SDS-PAGE gel used for quantification (FIG. 46A).

FIGS. 47A-47B show fluorescence photomicrographs showing mCherry protein expression within 3T3-D1 cells after transfection with either GP yRNA: 25k bPEI: mCherry mRNA control (FIG. 47A) or GP ensilicated mCherry mRNA (FIG. 47B). Upper panels show brightfield micrograph overlaid over mCherry fluorescence photomicrograph and lower panels show only mCherry fluorescence. Ensilicated samples were prepared with optimized method (10% APTES+90% TEOS) for ensilication of nucleic acids.

FIGS. 48A-48C show a schematic representation of different methods that can be used for adding an enteric coating polymer to YPs. YPs can be packed in commercially available enteric coated capsules (FIG. 48A); YPs can be individually coated in a hydrodynamic volume of enteric polymer (FIG. 48B); or agglomerated YPs can be coated with an enteric polymer (FIG. 48C).

### DETAILED DESCRIPTION

The present disclosure improves upon conventional encapsulation technologies by providing a yeast particle (YP) delivery system comprising an extracted yeast cell wall and an ensilicated payload.

Silica based trapping methods have been previously reported for many drug delivery applications. Mesoporous silica nanoparticles (MSN) formed by polymerization of tetraethylorthosilane (TEOS) around a sacrificial micelle frame have been extensively used for drug encapsulation. MSNs have been previously used for encapsulation of the chemotherapeutic doxorubicin (DOX). However, the chemical synthetic conditions needed for the formation of MSNs cannot be generated inside YPs. To overcome this synthetic hurdle, DOX delivery was attempted by non-covalently liking MSNs to the outer surface of YPs functionalized with anionic polymers. Although this approach worked, its drawbacks included limited payload loading capacity and poor stability of the MSN-DOX bound on the outer surface of YPs.

Encapsulation of proteins in silica-based “cages” prepared from tetraethylorthosilicate (TEOS) has been previously described by Chen et al. (Chen, Y-C, Smith, T, Hicks, R. H, Doekhie, A, Koumanov, F, Wells, S. A, Edler, K. J, Van Den Elsen, J, Holman, G. D, Marchbank, K. J, Sartbaeva, A. Tailored stability, storage and release of proteins with tailored fit in silica, Nature Scientific Reports, 2017, 7:46568) and Wahid et al. (Wahid, A. A., Doekhie, A., Sartbaeva, A. et al. Ensilication Improves the Thermal Stability of the Tuberculosis Antigen Ag85b and an Sbi-Ag85b Vaccine Conjugate. Sci Rep 9, 11409 (2019)). The protein-silica complex provides protection against heat denaturing of protein.

The use of TEOS and other silicates on yeast particles has been reported for the preparation of vaccines comprising YPs loaded with the vaccine and a polymerized silicate contacting the YP such that the YP is capped by the silicate (plug seal approach) (Wagner, T. E, Vaccine Delivery Systems Using Yeast Cell Wall Particles, 2017, U.S. Pat. No. 10,166,195, and United States Patent Application publication 2017/0007688 A1).

In the “ensilication” approach, a partially pre-polymerized TEOS is reacted with a payload leading to formation of a rigid silica shell surrounding the payload, thus protecting the payload from external factors that could lead to denaturing and/or degradation to form an insoluble complex. The reported procedure was developed to produce large ensilicated protein particles (average diameter >700 nanometers). Such large particles cannot be loaded inside YPs.

The subject disclosure is based in part on the discovery that partially pre-polymerized TEOS could be loaded inside YPs to trap payloads via ensilication in YPs. Dry YP loaded with payloads (e.g., nanoparticles, proteins, antibodies, nucleic acids, and the like) were incubated with partially pre-polymerized TEOS or other silane compounds. Partially pre-polymerized TEOS was then diffused into the hollow YPs and formed a silica shell around the payload. The payloads were trapped with high efficiency inside the silica shell in the YP cavity. The YP carrying ensilicated payloads could be phagocytosed by macrophage cells. The payload was efficiently released inside phagocytic cells. In addition to retaining biological activity of payload while offering similar or higher encapsulation efficiency as previous methods, the instant YP ensilication method offers improved payload stability due to higher resistance to thermal or enzymatic degradation.

In the present disclosure, YPs carrying payload molecules are incubated with partially pre-polymerized TEOS. Diffusion of TEOS into the YPs leads to ensilication of the payload inside the YPs such that the chemical or biologic activities of the payloads are not permanently altered or diminished. The compositions and methods of the present disclosure can yield a highly stable YP-payload delivery system with an increased resistance to thermal or enzymatic degradation, thereby, providing for a significant improvement over existing technology.

Other compounds can also be used for producing a silica shell. These include the tetraethylorthogermanate (TEOG), tetramethylorthosilicate (TMOS), aminopropyl tricthoxysilicate (APTES), 3-(tricthoxysylil)-propyl-isocyanate (TEPI), bis[3-tricthoxysilyl)propyl]disulfide (BTEPDS), bis[3-(tricthoxysilyl) propyl]tetrasulfide (BTEPTS) and a combination thereof. The sulfur-sulfur bonds in BTEPDS and PTEPTS allow for encapsulation of payloads in a silica shell with S—S bonds that are susceptible for cleavage by glutathione inside the cells. This allows for controlled release of payloads in the cells (faster release from payloads trapped in a silica shell composed of TEOS and BTEPDS than TEOS alone).

The disclosures of patents, patent applications, and publications in their entireties are hereby incorporated by reference into this application in order to more fully describe the state of the art as known to those skilled therein as of the date of the disclosure described and claimed herein (e.g., U.S. Pat. Nos. 7,740,861, 8,389,485, 9,242,857, 9,655,360, 10,004,229, 10,166,195; European Patent No. 1711058, WO2005070213A2, WO2005113128A1 and associated patents/patent applications). The instant disclosure will govern in the instance that there is any inconsistency between the patents, patent applications, and publications and this disclosure.

That the disclosure may be more readily understood, select terms are defined below.

The term “about” in connection with numerical values and ranges means that the number comprehended is not limited to the exact number set forth herein, and is intended to refer to ranges substantially within the quoted range while not departing from the scope of the disclosure. As used herein, “about” will be understood by persons of ordinary skill in the art. For example, “about” means that ±10% of a particular numerical value following the term.

As used herein, the term “ensilication” or “ensilicated” means that a polymeric structure, like a “mesh net,” covers or coats the payload such that payload inside the YPs is retained or entrapped within a polymeric structure (“cage”) formed by a silicate.

As used herein, the term “capping” or “capped” means that a polymeric structure, like a “mesh net,” covers or coats the YPs such that the payload loaded within the YPs is retained or entrapped within. The polymeric structure can be formed by a silicate.

### Yeast Particles

As used herein, a “yeast particle” (YP) refers to readily available, biodegradable, substantially spherical, hollow particles of about 2-4 μm in diameter. YPs may be obtained as a byproduct of some food grade Baker's yeast (i.e., Saccharomyces cerevisiae) extract manufacturing processes. YPs include, but are not limited to, commercially available YPs (for example, BIORIGIN® and SAFMANNAN®), extracted yeast cell wall particles (YCWPs), yeast cell particles (YCPs), glucan particles (GPs), yeast glucan particles (YGPs), yeast glucan-mannan particle (YGMP), glucan lipid particles (GLPs), whole glucan particles (WGPs), glucan mannan lipid particles (GMLPs) and the like. YPs comprise a shell composed of remnants of cell wall components after yeast cell extraction and a large a hollow inner cavity. Methods of preparing extracted yeast cell wall particles are known in the art, and are described, for example in U.S. Pat. Nos. 4,992,540, 5,082,936, 5,028,703, 5,032,401, 5,322,841, 5,401,727, 5,504,079, 5,968,811, 6,444,448, 6,476,003, published U.S. applications 2003/0216346 A1, 2004/0014715 A1, 2021/0023017 A1, and PCT published application WO 02/12348 A2, which are specifically incorporated herein by reference.

### Hydration of Yeast Particles

A sufficient level of hydration of YPs is needed for encapsulation and release of payloads.

Dry YPs can be hydrated by incubation with a variety of aqueous solutions. Suitable aqueous solutions include, but are not limited to: water; saline, e.g., phosphate buffered saline; any buffer solution known in the art with a pH between 3 and 11; any acid solution known in the art with a pH≥1.5; any basic solution known in the art with a pH <11; any salt solution known in the art that does not chemically interfere with the payload, and the like.

### Payload Molecules

The nano-silica YPs of the present disclosure are useful for in vivo or in vitro delivery of payload molecules to a cell or an organism. Any molecular payload that can be ensilicated within the YP is envisioned by the present disclosure.

Payload can be a protein, a peptide, a nucleic acid, a polysaccharide or a combination thereof.

### A. Antimicrobial Payloads

Certain exemplary embodiments of the present disclosure provide for compositions and methods for the loading and delivery of hydrophobic payload molecules antimicrobial peptides effective against classes of organisms such as Gram positive bacteria, Gram negative bacteria, fungi, and viruses.

A peptide with microbicidal or microbiostatic inhibitory properties can be applied to an environment either presently exhibiting microbial growth (i.e., therapeutic treatment) or to an environment at risk of supporting such growth (i.e., prevention or prophylaxis). An environment capable of sustaining microbial growth refers to a fluid, substance, or organism where microbial growth can occur or where microbes can exist. Such environments can be, for example, animal tissue or bodily fluids, water and other liquids, food, food products or food extracts, crops, and certain inanimate objects. It is not necessary that the environment promote the growth of the microbe, only that it permit its subsistence.

The antimicrobial peptide component may comprise a single microbial or a mixture of antimicrobials.

Controlled release pharmaceutical dosage forms can be used to optimize drug delivery and enhance patient compliance. A pharmaceutical dosage form can deliver more than one drug, each at a modified rate.

### B. Other Payloads

The YP delivery system of the present disclosure is useful for in vivo or in vitro delivery of payload molecules including, but limited to, polynucleotides such as oligonucleotides, antisense constructs, siRNA, enzymatic RNA, and recombinant DNA constructs, including expression vectors.

In other exemplary embodiments, the YP delivery system of the present disclosure is useful for in vivo or in vitro delivery of payload molecules such as amino acids, peptides and proteins. By “protein” is meant a sequence of amino acids for which the chain length is sufficient to produce the higher levels of tertiary and/or quaternary structure. This is to distinguish from “peptides” or other small molecular weight drugs that do not have such structure. Typically, the protein herein will have a molecular weight of at least about 15-20 kD, or at least about 20 kD.

Examples of proteins encompassed within the definition herein include: mammalian proteins, such as, e.g., growth hormone (GH), including human growth hormone, bovine growth hormone, and other members of the GH supergene family; growth hormone releasing factor; parathyroid hormone; thyroid stimulating hormone; lipoproteins; alpha-1-antitrypsin; insulin A-chain; insulin B-chain; proinsulin; follicle stimulating hormone; calcitonin; luteinizing hormone; glucagon; clotting factors such as factor VIIIC, factor IX tissue factor, and von Willebrand's factor; anti-clotting factors such as Protein C; atrial natriuretic factor; lung surfactant; a plasminogen activator, such as urokinase or tissue-type plasminogen activator (t-PA); bombazine; thrombin; alpha tumor necrosis factor, beta tumor necrosis factor; enkephalinase; RANTES (regulated on activation normally T-cell expressed and secreted); human macrophage inflammatory protein (MIP-1-alpha); serum albumin such as human serum albumin; mullerian-inhibiting substance; relaxin A-chain; relaxin B-chain; prorelaxin; mouse gonadotropin-associated peptide; DNase; inhibin; activin; vascular endothelial growth factor (VEGF); receptors for hormones or growth factors; an integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-beta; platelet-derived growth factor (PDGF); fibroblast growth factor such as aFGF and bFGF; epidermal growth factor (EGF); transforming growth factor (TGF) such as TGF-alpha and TGF-beta, including TGF-beta1, TGF-beta2, TGF-beta3, TGF-beta4, or TGF-beta5; insulin-like growth factor-I and -II (IGF-I and IGF-II); des (1-3)-IGF-I (brain IGF-D; insulin-like growth factor binding proteins; CD proteins such as CD3, CD4, CD8, CD19 and CD20; osteoinductive factors; immunotoxins; a bone morphogenetic protein (BMP); T-cell receptors; surface membrane proteins; decay accelerating factor (DAF); a viral antigen such as, for example, a portion of the AIDS envelope; transport proteins; homing receptors; addressins; regulatory proteins; immunoadhesins; antibodies; and biologically active fragments or variants of any of the above-listed polypeptides.

The members of the GH supergene family include growth hormone, prolactin, placental lactogen, erythropoietin, thrombopoietin, interleukin-2, interleukin-3, interleukin-4, interleukin-5, interleukin-6, interleukin-7, interleukin-9, interleukin-10, interleukin-11, interleukin-12 (p35 subunit), interleukin-13, interleukin-15, oncostatin M, ciliary neurotrophic factor, leukemia inhibitory factor, alpha interferon, beta interferon, gamma interferon, omega interferon, tau interferon, granulocyte-colony stimulating factor, granulocyte-macrophage colony stimulating factor, macrophage colony stimulating factor, cardiotrophin-1 and other proteins identified and classified as members of the family.

The protein payload molecule is typically essentially pure and desirably essentially homogeneous (i.e. free from contaminating proteins etc.). “Essentially pure” protein means a composition comprising at least about 90% by weight of the protein, based on total weight of the composition, or at least about 95% by weight. “Essentially homogeneous” protein means a composition comprising at least about 99% by weight of protein, based on total weight of the composition. Proteins may be derived from naturally occurring sources or produced by recombinant technology. Proteins include protein variants produced by amino acid substitutions or by directed protein evolution (Kurtzman, A. L., et al., Advances in directed protein evolution by recursive genetic recombination: applications to therapeutic proteins, Curr Opin Biotechnol. 2001 12 (4): 361-70) as well as derivatives, such as PEGylated proteins.

In certain embodiments, the protein is an antibody or an antigen-binding fragment thereof. The antibody or antigen-binding fragment thereof may bind to any of the above-mentioned molecules, for example. Exemplary molecular targets for antibodies encompassed by the present disclosure include but are not limited to: CD proteins such as CD3, CD4, CD8, CD19, CD20 and CD34; members of the HER receptor family such as the EGF receptor, HER2, HER3 or HER4 receptor; cell adhesion molecules such as LFA-1, Mol, p150,95, VLA-4, ICAM-1, VCAM and alphav/beta3 integrin including either alpha or beta subunits thereof (e.g. anti-CD11a, anti-CD18 or anti-CD11b antibodies); growth factors such as VEGF; and the like.

Other active agents that can be incorporated in the delivery system of the present disclosure include: gastrointestinal therapeutic agents such as aluminum hydroxide, calcium carbonate, magnesium carbonate, sodium carbonate and the like; digestants, enzymes and the like.

### Nano-Silica Cage

In certain embodiments, at least one payload of a YP is substantially encapsulated by a nano-silica cage. As used herein, a “nano-silica cage” refers to a covalently bonded silica matrix that surrounds a payload without chemically interacting with the payload, wherein the shape of the silica matrix closely matches the shape of the payload and provides a physical barrier to release of the payload from the silica matrix. In certain embodiments, a payload is “substantially encapsulated” by a nano-silica cage if it is about 50% to about 100% encapsulated. For example, the payload can be about 50%, about 55%, about 65%, about 70%, about 75%, about 80%, about 90%, about 91%, about 92%, about 93%, about 94%, about 95%, about 96%, about 97%, about 98%, about 99%, or about 100% encapsulated by a nano-silica cage.

### Payload Encapsulation Efficiency and Encapsulation Yield

Two parameters used to evaluate the payload encapsulation process are encapsulation yield (EY) and encapsulation efficiency (EE). EY, expressed as a percent value, is the weight ratio between payload and YP. EE, expressed as a percent value, is the weight ratio of the payload loaded or encapsulated in a YP with respect to the payload's initial mass. High EE is desirable to maximize payload delivery. In certain embodiments, EE can range from about 50% to about 100%. For example, the payload encapsulation efficiency can be about 50%, about 55%, about 65%, about 70%, about 75%, about 80%, about 90%, about 91%, about 92%, about 93%, about 94%, about 95%, about 96%, about 97%, about 98%, about 99%, or about 100%.

### Vaccines

In exemplary embodiments, the YP delivery system of the present disclosure is useful in providing oral delivery of vaccines. In exemplary embodiments, the system is used to deliver antigens, such as antigens of such microorganisms as Neisseria gonorrhea, Mycobacterium tuberculosis, Herpes virus (humonis, types 1 and 2), Candida albicans, Candida tropicalis, Trichomonas vaginalis, Haemophilus vaginalis, Group B Streptococcus sp., Microplasma hominis, Hemophilus ducreyi, Granuloma inguinale, Lymphopathia venereum, Treponema pallidum, Brucella abortus. Brucella melitensis, Brucella suis, Brucella canis, Campylobacter fetus, Campylobacter fetus intestinalis, Leptospira pomona, Listeria monocytogenes, Brucella ovis, equine herpes virus 1, equine arteritis virus, IBR-IBP virus, BVD-MB virus, Chlamydia psittaci, Trichomonas foetus, Toxoplasma gondii, Escherichia coli, Actinobacillus equuli, Salmonella abortus ovis, Salmonella abortus equi, Pseudomonas aeruginosa, Corynebacterium equi, Corynebacterium pyogenes, Actinobaccilus seminis, Mycoplasma bovigenitalium, Aspergillus fumigatus, Absidia ramosa, Trypanosoma equiperdum, Babesia caballi, Clostridium tetani, Clostridium botulinum and the like. In other embodiments, the system can be used to deliver neutralizing antibodies that counteract the above microorganisms.

In other embodiments, the system can be used to deliver enzymes such as ribonuclease, neuraminidase, trypsin, glycogen phosphorylase, sperm lactic dehydrogenase, sperm hyaluronidase, adenosinetriphosphatase, alkaline phosphatase, alkaline phosphatase esterase, amino peptidase, trypsin chymotrypsin, amylase, muramidase, acrosomal proteinase, diesterase, glutamic acid dehydrogenase, succinic acid dehydrogenase, beta-glycophosphatase, lipase, ATP-ase alpha-peptate gamma-glutamylotranspeptidase, sterol-3-beta-ol-dehydrogenase, DPN-diaphorase, glucocerebrosidase and other lysosomal hydrolases used for enzyme replacement therapies.

In exemplary embodiments, the system can deliver antigens of bioterrorism critical biological agents, including Category A agents such as variola major (smallpox), Bacillus anthracis (anthrax), Yersinia pestis (plague), Clostridium botulinum toxin (botulism), Francisella tularensis (tularaemia), filoviruses (Ebola hemorrhagic fever, Marburg hemorrhagic fever), arenaviruses (Lassa (Lassa fever), Junin (Argentine hemorrhagic fever) and related viruses); Category B agents such as Coxiella burnetti (Q fever), Brucella species (brucellosis), Burkholderia mallei (glanders), alphaviruses (Venezuelan encephalomyelitis, castern & western equine encephalomyelitis), ricin toxin from Ricinus communis (castor beans), epsilon toxin of Clostridium perfringens; Staphylococcus enterotoxin B, Salmonella species, Shigella dysenteriae, Escherichia coli strain 0157: H7, Vibrio cholerae, Cryptosporidium parvum; and Category C agents such as nipah virus, hantaviruses, tickborne hemorrhagic fever viruses, tickborne encephalitis viruses, yellow fever, and multidrug-resistant tuberculosis.

In exemplary embodiments, the system can be used to deliver inactivated antigenic toxins, such as anatoxin antigens, including toxoids (inactivated but antigenic toxins), and toxoid conjugates. In exemplary embodiments, the toxoid is an inactivated microbial toxin. In other embodiments, the toxoid is an inactivated plant toxin. In further embodiments, the toxoid is an inactivated animal toxin. In certain embodiments, the system can be used to deliver toxoids such as pertussis toxoid, Corynebacterium diphtheriae toxoid, tetanus toxoid, Haemophilus influenzae type b-tetanus toxoid conjugate, Clostridium botulinum D toxoid, Clostridium botulinum E toxoid, toxoid produced from Toxin A of Clostridium difficile, Vibrio cholerae toxoid, Clostridium perfringens Types C and D toxoid, Clostridium chauvoei toxoid, Clostridium novyi (Type B) toxoid, Clostridium septicum toxoid, recombinant HIV tat IIIB toxoid, Staphylococcus toxoid, Actinobacillus pleuropneumoniae Apx I toxoid, Actinobacillus pleuropneumoniae Apx II toxoid, Actinobacillus pleuropneumoniae Apx III toxoid, Actinobacillus pleuropneumoniae outer membrane protein (OMP) toxoid, Pseudomonas aeruginosa clastase toxoid, snake venom toxoid, ricin toxoid, Mannheimia haemolytica toxoid, Pasteurella multocida toxoid, Salmonella typhimurium toxoid, Pasteurella multocida toxoid, and Bordetella bronchiseptica toxoid.

### Solvents, Loading Solvent, and Leave-in Solvents

Solvents may be added during the encapsulation process to facilitate loading of payloads in the YPs. Certain payloads of the present disclosure are water-insoluble or have low water solubility and may be loaded into YPs with a solvent that is compatible with yeast particles. In certain nonlimiting embodiments, the solvent may be an organic solvent. Suitable solvents include, but are not limited to, be acetone, dichloromethane, ethyl acetate, alcohols such as ethanol or methanol, dimethylsulfoxide (DMSO), methanol-chloroform, hexane, petroleum ether, toluene, Neobee and the like. After a payload is completely encapsulated, the yeast particle and payloads may be processed to remove the solvent from the YP-payload formulation. Organic solvents such as acetone, dichloromethane, ethyl acetate, methanol, and DMSO may be unsafe for human administration and should be removed after a payload is completely encapsulated. Alternatively, the solvent used to facilitate payload encapsulation may be safe for human administration and can be left inside the YP along with the water-insoluble payload as a “leave-in solvent.”

### Surfactants

The term “surfactant,” as used herein, refers to any molecule having both a hydrophilic group (e.g., a polar group), which energetically prefers solvation by water, and a hydrophobic group which is not well solvated by water. The term “nonionic surfactant” is a known term in the art and generally refers to a surfactant molecule whose hydrophilic group (e.g., polar group) is not electrostatically charged.

Surfactants are generally low to moderate weight compounds which contain a hydrophobic portion, which is generally readily soluble in oil, but sparingly soluble or insoluble in water, and a hydrophilic portion, which is sparingly soluble or insoluble in oil, but readily soluble in water. In addition to protecting against growth and aggregation and stabilizing the organic compound delivery vehicle, surfactants are also useful as excipients in organic compound delivery systems and formulations because they increase the effective solubility of an otherwise poorly soluble or non-soluble organic compound, and may decrease hydrolytic degradation, decrease toxicity and generally improve bioavailability. Surfactants may also provide selected and advantageous effects on drug release rate and selectivity of drug uptake. Surfactants are generally classified as either anionic, cationic, or nonionic.

Suitable surfactants include, but are not limited to, sodium lauryl sulphate, polysorbate 20, polysorbate 80, polysorbate 40, polysorbate 60, polyglyceryl ester, mono fatty acid ester of polyoxyethylene sorbitan, polyglyceryl monooleate, decaglyceryl monocaprylate, propylene glycol dicaprilate, polyethylenepolypropylene glycol, triglycerol monostearate, polyoxyethylenesorbitan, monooleate, Tween®, Span® 20, Span® 40, Span® 60, Span® 80, IGEPAL®, Triton X-100, Neobec Brij 30 and the like, and any mixtures thereof.

### Temperature Stabilizing Agents

The storage stability of YPs containing payloads may be improved by addition of one or more temperature stabilizing agents. Common temperature stabilizing agents include sugars such sucrose, trehalose, glycerol, or sorbitol. Disaccharides such as sucrose and trehalose are natural cryoprotectants with good protective properties. A temperature stabilizing agent may comprise a carbohydrate component including between about 10% and 80% oligosaccharide, between about 5% and 30% disaccharide or between about 1% and 10% polysaccharide, and a protein component including between about 0.5% and 40% protein, e.g., hydrolyzed animal or plant proteins, based on the total weight of the composition. Ascorbic acid ions may be used in some embodiments for stabilization at higher temperature and humidity exposure. Alternatively, a combination of citrate and/or ascorbate ions with protein or protein hydrolysate may be used. In certain nonlimiting embodiments, the temperature stabilizing agent may be a glycerin. In certain nonlimiting embodiment temperature stabilizing agent may be glycerin at a concentration of about 5%, about 10%, about 15%, about 20%, about 25%, about 30%, about 35%, about 40%, about 45% or about 50%. In certain nonlimiting embodiment temperature stabilizing agent may be glycerin at a concentration of 1-5%, 5-10%, 10-15%, 15-20%, 20-25%, 25-30%, 35-40%, 40-45%, or 45%-50%.

### Derivatization of Yeast Particle Surface for Improved Uptake

The oral administration of drugs and vaccines is often limited by the fast degradation of drugs in the stomach. GPs are transported across the small intestine via specialized M-cells located in Peyer's Patches (PP), which constitute less than 1% of the intestinal epithelium. As specialized enterocytes, M-cells are located throughout the follicle-associated epithelium (FAE), a single layer of epithelial cells that separates the external luminal environment from the immune cells within the PP. M-cells allow for the controlled and effective sampling of luminal contents making PPs the immune sensors and inductive sites of the small intestine. Below the FAE, in the subepithelial dome (SED) region, there is an extensive network of phagocytes that are postulated to be involved in the active sampling of antigens following M-cell transport. Following M-cell-mediated transepithelial transport, GPs accumulate in a subset of SED CD11c+ DCs distinguished from other PP DC subsets by their expression of Langerin, a C-type lectin receptor that plays a role in pathogen recognition.

In the gut associated lymphatic tissue, particle uptake by phagocytosing immune cells is mediated by receptors that recognize β-1,3-D-glucan, such as Dectin-1 (D1) and Complement Receptor 3 (CR3). Similarly, recognition of specific ligands by cell surface receptors localized in intestinal epithelial cells leads to the transport of materials across the targeted biological barrier, towards the bloodstream. Thus, targeting surface derivatized GPs to cells other than PP M-cells can increase the intestinal uptake of GPs. An enhanced GP absorption by the intestines makes them promising vehicles for the oral delivery of vaccines, insulin, monoclonal antibodies and other drugs that are currently administered intravenously or have low bioavailability.

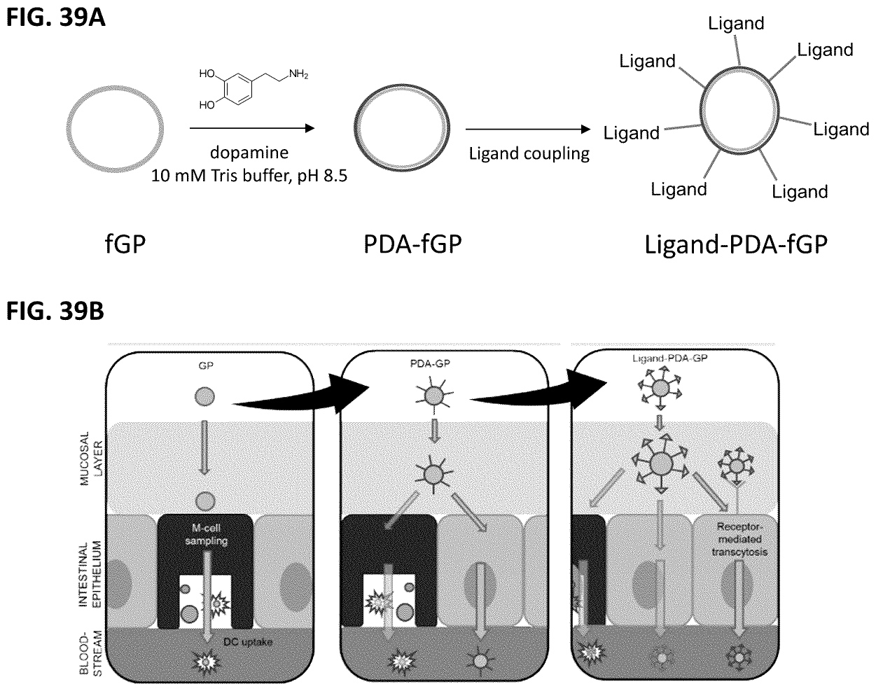

One approach to functionalize surfaces is based on the polymerization of dopamine on the surface. Film coatings can be obtained by polymerization of dopamine hydrochloride (DOPA) under mild basic conditions (see FIG. 39A). Polymerization of DOPA allows the incorporation of several functional groups on the coated surface (catechol, amine, and imine) that can serve as starting points for additional surface modifications such as ligand coupling. PDA coating is known to enhance mucopenetration and in vitro cell uptake of polystyrene nanoparticles (Poinard et al., 2019). As schematically represented in FIG. 39B, PDA-coated YPs can enhance transport across the intestinal epithelium following oral administration in mice. PDA coating of YPs provides for a novel approach to further develop GPs for oral drug delivery.

### Enhanced Mucus/Mucin Penetration

Mucus is a viscoelastic, adhesive gel that coats all exposed epithelial surfaces not covered by skin, such as those of the respiratory, gastrointestinal (GI), and female reproductive tracts, and the surface of the eye. Mucus protects the underlying epithelium by both lubricating the surface and trapping and removing foreign particulates. Mucus is an effective barrier to sustained local drug delivery. Large drug-releasing particles become trapped in the upper layers of the mucus and are rapidly eliminated; small drug molecules pass readily through the mucus but are then quickly absorbed into the circulatory system and removed from the locally diseased tissue. Penetrating deep into the mucus barrier without compromising its protective properties could lead to improved prophylactic and therapeutic treatments for diseases. Coating particles that improve mucus/mucin penetration significantly improve the particle's diffusion through mucus. Substances that have mucus/mucin penetrating properties include polydopamine (PDA), chitosan, poly(acrylic acid), alginate, poly(methacrylic acid) and sodium carboxymethyl cellulose.

### Addition of Cell Surface Receptor Ligands

More intestinal cells can be recruited for YP uptake, by attaching ligands known to target receptors at the intestinal epithelial lining are added to YP surface. PDA coating provides primary amines at the surface of YPs which are useful for the attachment of various ligands by reaction with their carboxylic acid functionalities. FIG. 39A shows a schematic for the surface derivatization/modification of YPs with receptor ligands. Some ligands are known to target receptors found on enterocytes and goblet cells, which greatly outnumber M-cells in the intestinal epithelium. Thus, the use of these ligand-derivatized PDA-YPs can provide a more efficient vehicle for the oral delivery of vaccines and drugs inside YPs, targeting the intestinal epithelial barrier. FIG. 39B shows the schematic representation of enhanced uptake of ligand-derivatized PDA-YPs in the intestine.

### Enteric Coating Polymers

Pharmaceutical dosage forms or preparations with enteric coating are well known in the art. The typical purpose of using such enteric coatings is protecting acid sensitive drugs from gastric acid and/or protecting the gastric mucosa from irritation or damage resulting from drugs that are not well-tolerated when released in the stomach. When realizing such enteric coatings, usually the physical and physiological environment in the human digestive tracts has to be considered, as well as the passing time in the gastrointestinal tracts. In the gastrointestinal tracts of healthy persons, the physiological pH changes usually from acidic (pH of from about 1.8 to 4.5) to neutral (pH of from about 6.5 to 8.0) from the stomach to the small intestine and no difference is presumed to exist in the physiological environment between the small intestine and large intestine. The retention time of the preparation in the human stomach is from 0.5 to 10 hours and significantly depends on the individual person, a concurrent food intake and the size of the preparation to be administered. On the other hand, the fluctuations of a passing time through the small intestine are relatively small and the passing time is said to be 3+/−1 hours (h) (Journal of Controlled Release, 2, 27-38 (1985)).

Enteric coatings are well known in the art. An enteric coating, also known as gastro-resistant coating is a barrier applied to oral medication that controls the location in the digestive tract where it is absorbed. The term “enteric” refers to the small intestine; therefore, enteric coatings resist breakdown of medication before it reaches the small intestine. Enteric coatings are employed when the drug substance is inactivated or destroyed in the acid secretion of the stomach or is particularly irritating to the gastric mucosa or when bypass of the stomach substantially enhances drug absorption.

For instance, the commercially available enteric coating polymers sold under the tradename EUDRAGIT® (Degussa, Germany) do not dissolve in the stomach. Accordingly, EUDRAGIT®S 100 dissolves at a pH value above 7.0 which corresponds to the conditions as present in the colon. This leads to the effect that the enteric coating does not dissolve in the stomach, but in the colon. A further example of an enteric coating polymer is EUDRAGIT® L100-55, which dissolves at a pH value above 5.5, which means that it dissolves in the duodenum.

Modern enteric coatings are usually formulated with synthetic polymeric material often referred to as polyacids. These polymers contain ionizable functional groups that render them water-soluble at a specific pH value. Examples of coating polymers include methyl acrylate, methyl methacrylate, methacrylic acid, cellulose acetate phthalate (CAP), cellulose acetate trimellate (CAT), hydroxy propyl methyl cellulose phthalate (HPMCP), hydroxyl propyl methyl cellulose acetate succinate (HPMCAS), and polyvinyl acetate (PVAP). Several different types of Eudragit polymers with enteric release capabilities are commercially available in a wide range of different physical forms (aqueous dispersion, organic solution, granules and powders). The pH at which these polymers dissolve is dependent on the content of the carboxylic acid in the copolymer. Methacrylic acid methylmethacrylate copolymers (Eudragit L and S), and methacrylic acid ethyl acrylate copolymer (Eudragit L30D) are the exemplary choice of coating polymers for enteric formulations. They allow targeting of specific areas of the intestine.

The term “gastric resistant” refers to a composition whose bioactivity is affected as little as possible under in-vitro conditions that mimic the conditions of a vertebrate stomach, i.e., pH 1.2 for at least about 2 hours. Gastric resistance is imparted to the composition by a coating layer (enteric coating layer) which reduces or prevents the release of the ingredients in the composition during exposure to stomach acids. Gastric resistance ensures that acid-sensitive ingredients in the composition are protected against inactivation and that ingredients in the composition which may be irritate the stomach mucosa are not set free in too high amounts. Upon entering the intestine, the enteric coating layer dissolves at the slightly higher intestinal pH (˜6.8) thereby releasing/exposing the contents of the composition to the intestinal environment.

### Articles of Manufacture, Compositions, and Methods

Certain embodiments of the present disclosure provide compositions and methods for use in controlling sucking and biting pests, including e.g., mosquitoes, ticks, lice, fleas, mites, flies, and spiders.

Certain embodiments of the present disclosure provide for compositions and methods for use in controlling nematodes. Nematodes (Kingdom: Animalia; Phylum: Nematoda) are microscopic round worms. They can generally be described as aquatic, triploblastic, unsegmented, bilaterally symmetrical roundworms, that are colorless, transparent, usually bisexual, and worm-shaped (vermiform), although some can become swollen (pyroform).

Many nematodes are obligate parasites and a number of species constitute a significant problem in agriculture. Thus, methods to control their parasitic activities are an important feature in maximizing crop production in modern intensive agriculture.

Nematodes are not just parasitic to plants but a number of species are parasitic to animals, both vertebrate and invertebrate. Around 50 species attack humans and these include Hookworm (Anclyostoma), Strongylids (Strongylus), Pinworm (Enterolobius), Trichinosis (Trichina), Elephantitis (Wuchereria), Heartworm (Dirofilaria), and Ascarids (Ascaris).

In some embodiments of the present disclosure, any of the compositions described above may be formulated in a deliverable form suited to a particular application. Deliverable forms that can be used in accordance with embodiments of the present disclosure include, but are not limited to, liquids, emulsions, emulsifiable concentrates, solids, aqueous suspensions, oily dispersions, pastes, granules, powders, dusts, fumigants, and aerosol sprays. Suitable deliverable forms can be selected and formulated by those skilled in the art using methods currently known in the art. The compositions can be provided in combination with an agriculturally, food, or pharmaceutically acceptable carrier or excipient in a liquid, solid, or gel-like form. For solid compositions, suitable carriers include pharmaceutical or food grades of mannitol, lactose, starch, magnesium stearate, sodium saccharin, talc, cellulose, glucose, sucrose, and magnesium carbonate. Suitably, the formulation is in tablet or pellet form. As suitable carrier could also be a human or animal food material. Additionally, conventional agricultural carriers could also be used.

In some embodiment of the present disclosure, payload delivery strategies include placing the YP payload in an enteric capsule, agglomerating in a disintegrating matrix, and coat granules or compress into a tablet and enteric coat tablet, etc.

The composition of the present disclosure may alternatively be applied via irrigation. This is suitable for treating nematodes or other soil borne pathogens or parasites.

In certain embodiments, the present disclosure provides for compositions in the form of granules and methods of controlling pests using the same. Granules allow for the use of less selective herbicides, pesticides, and combinations thereof, and thus offer a means to control pests that are not otherwise easily controlled. Granules are a convenient application form for producers with small allotments such as paddy rice farmers, or for growers of turf where spays are complicated by the needs of near neighbors sensitive to drift or odor or for broad acre farmers who wish to apply fertilizers and herbicides together and who do not have convenient access to water.

The granules may be used in flooded paddies, recently irrigated turf, or in areas where it is inconvenient or impossible to remove irrigation water. The granules allow small holders the means to apply crop protection chemicals without expensive equipment, and without risk of exposing airways or eyes to aerosols or spray materials. Granules can be easily measured and distributed by hand. Using granules that are designed for uniform dispersal is advantageous because it ensures even application, prevents post-harvest decay, and allows coating of seeds.

### Pharmaceutical Compositions and Administration

In addition, the compositions and methods of the present disclosure are useful in the fields of industrial and consumer products and medicines, e.g., in food, human and animal drugs, and cosmetics, and the like. In some embodiments, the disclosure provides for compositions and methods for use in both human and veterinary medicine. In certain embodiments, the present disclosure relates to therapeutic treatment of mammals, birds, and fish. For example, the compositions and methods of the present disclosure are useful for therapeutic treatment of mammalian species including, but not limited to, human, bovine, ovine, porcine, equine, canine, and feline species.

Routes of administration of the delivery system include but are not limited to oral, buccal, sublingual, pulmonary, transdermal, transmucosal, as well as subcutaneous, intraperitoneal, intravenous, and intramuscular injection. Exemplary routes of administration are oral, buccal, sublingual, pulmonary, and transmucosal.

The YP delivery system of the present disclosure is administered to a patient in a therapeutically effective amount. The YPs can be administered alone or as part of a pharmaceutically acceptable composition. In addition, a compound or composition can be administered all at once, as for example, by a bolus injection, multiple times, such as by a series of tablets, or delivered substantially uniformly over a period of time, as for example, using a controlled release formulation. It is also noted that the dose of the compound can be varied over time. The YP delivery system can be administered using an immediate release formulation, or using a controlled release formulation, or combinations thereof. The term “controlled release” includes sustained release, delayed release, and combinations thereof, as well as release mediated by chemical (e.g., pH) and/or biological (e.g., enzyme) hydrolysis.

A pharmaceutical composition of the disclosure can be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient that would be administered to a patient or a convenient fraction of such a dosage such as, for example, one-half or one-third of such a dosage.

The relative amounts of the active ingredient, the pharmaceutically acceptable carrier, and any additional ingredients in a pharmaceutical composition of the disclosure will vary, depending upon the identity, size, and condition of the animal or human treated, and further depending upon the route by which the composition is to be administered. By way of example, the composition can comprise between 0.1% and 100% (w/w) active ingredient. For example, the active ingredient weight in the pharmaceutical composition may be at least about 1%, 5%, 10%, 15%, 20%, 25%, 30%, 35%, 40%, 45%, 50%, 55%, 60%, 65%, 70%, 75%, 80%, 85%, 90%, 95%, or 100%. A unit dose of a pharmaceutical composition of the disclosure will generally comprise from about 100 milligrams to about 2 grams of the active ingredient, or from about 200 milligrams to about 1.0 gram of the active ingredient.

In addition, YP delivery system of the present disclosure can be administered alone, in combination with YPs with a different payload, or with other pharmaceutically active compounds. The other pharmaceutically active compounds can be selected to treat the same condition as the YPs with ensilicated payloads or a different condition.

If the patient is to receive or is receiving multiple pharmaceutically active compounds, the compounds can be administered simultaneously or sequentially in any order. For example, in the case of tablets, the active compounds may be found in one tablet or in separate tablets, which can be administered at once or sequentially in any order. In addition, it should be recognized that the compositions can be different forms. For example, one or more compounds may be delivered via a tablet, while another is administered via injection or orally as a syrup.

Another aspect of the disclosure relates to a kit comprising a pharmaceutical composition of the disclosure and instructional material. Instructional material includes a publication, a recording, a diagram, or any other medium of expression which is used to communicate the usefulness of the pharmaceutical composition of the disclosure for one of the purposes set forth herein in a human. The instructional material can also, for example, describe an appropriate dose of the pharmaceutical composition of the disclosure. The instructional material of the kit of the disclosure can, for example, be affixed to a container which contains a pharmaceutical composition of the disclosure or be shipped together with a container which contains the pharmaceutical composition. Alternatively, the instructional material can be shipped separately from the container with the intention that the instructional material and the pharmaceutical composition be used cooperatively by the recipient.

The disclosure also includes a kit comprising a pharmaceutical composition of the disclosure and a delivery device for delivering the composition to a human. By way of example, the delivery device can be a squeezable spray bottle, a metered-dose spray bottle, an aerosol spray device, an atomizer, a dry powder delivery device, a self-propelling solvent/powder-dispensing device, a syringe, a needle, a tampon, or a dosage-measuring container. The kit can further comprise an instructional material as described herein.

For example, a kit may comprise two separate pharmaceutical compositions comprising respectively a first composition comprising a particulate delivery system and a pharmaceutically acceptable carrier; and composition comprising second pharmaceutically active compound and a pharmaceutically acceptable carrier. The kit also comprises a container for the separate compositions, such as a divided bottle or a divided foil packet. Additional examples of containers include, without limitation, syringes, boxes, and bags. Typically, a kit comprises directions for the administration of the separate components. The kit form is advantageous when the separate components are administered in different dosage forms (e.g., oral and parenteral), are administered at different dosage intervals, or when titration of the individual components of the combination is desired by the prescribing physician.

An example of a kit is a blister pack. Blister packs are well known in the packaging industry and are being widely used for the packaging of pharmaceutical unit dosage forms, e.g., tablets and capsules. Blister packs generally consist of a sheet of relatively stiff material covered with a foil of, e.g., a transparent plastic material. During the packaging process, recesses are formed in the plastic foil. The recesses have the size and shape of the tablets or capsules to be packed. Next, the tablets or capsules are placed in the recesses and a sheet of relatively stiff material is sealed against the plastic foil at the face of the foil which is opposite from the direction in which the recesses were formed. As a result, the tablets or capsules are sealed in the recesses between the plastic foil and the sheet. The strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen that the tablets or capsules so specified should be ingested. Another example of such a memory aid is a calendar printed on the card, e.g., as follows “First Week, Monday, Tuesday, . . . etc. . . . , Second Week, Monday, Tuesday,” etc. Other variations of memory aids will be readily apparent.

Dosing can be hourly, e.g., every hour, every two hours, every four hours, every eight hours etc. Dosing can be weekly, biweekly, every four weeks, etc. A “daily dose” can be a single tablet or capsule or several pills or capsules to be taken on a given day. Also, a daily dose of a particulate delivery system composition can consist of one tablet or capsule, while a daily dose of the second compound can consist of several tablets or capsules and vice versa. The memory aid should reflect this and assist in correct administration.

In another embodiment of the present disclosure, a dispenser designed to dispense the daily doses one at a time in the order of their intended use is provided. The dispenser may be equipped with a memory aid, so as to further facilitate compliance with the dosage regimen. An example of such a memory aid is a mechanical counter, which indicates the number of daily doses that have been dispensed. Another example of such a memory aid is a battery-powered micro-chip memory coupled with a liquid crystal readout, or audible reminder signal which, for example, reads out the date that the last daily dose has been taken and/or reminds one when the next dose is to be taken.

A YP delivery system composition, optionally comprising other pharmaceutically active compounds, can be administered to a patient either orally, rectally, parenterally, (for example, intravenously, intramuscularly, or subcutaneously) intracisternally, intravaginally, intraperitoneally, intravesically, locally (for example, powders, ointments or drops), or as a buccal or nasal spray.

Parenteral administration of a pharmaceutical composition includes any route of administration characterized by physical breaching of a tissue of a human and administration of the pharmaceutical composition through the breach in the tissue. Parenteral administration thus includes administration of a pharmaceutical composition by injection of the composition, by application of the composition through a surgical incision, by application of the composition through a tissue-penetrating non-surgical wound. Parenteral administration includes subcutaneous, intraperitoneal, intravenous, intraarterial, intramuscular, or intrasternal injection and intravenous, intraarterial, or kidney dialytic infusion techniques.

Compositions suitable for parenteral injection comprise the active ingredient combined with a pharmaceutically acceptable carrier such as physiologically acceptable sterile aqueous or nonaqueous solutions, dispersions, suspensions, or emulsions, or may comprise sterile powders for reconstitution into sterile injectable solutions or dispersions. Examples of suitable aqueous and nonaqueous carriers, diluents, solvents, or vehicles include water, isotonic saline, ethanol, polyols, e.g., propylene glycol, polyethylene glycol, and glycerol, and suitable mixtures thereof, triglycerides, including vegetable oils such as olive oil, or injectable organic esters such as ethyl oleate. Proper fluidity can be maintained, for example, by the use of a coating such as lecithin, by the maintenance of the required particle size in the case of dispersions, and/or by the use of surfactants. Such formulations can be prepared, packaged, or sold in a form suitable for bolus administration or for continuous administration. Injectable formulations can be prepared, packaged, or sold in unit dosage form, such as in ampules, in multi-dose containers containing a preservative, or in single-use devices for auto-injection or injection by a medical practitioner.

Formulations for parenteral administration include suspensions, solutions, emulsions in oily or aqueous vehicles, pastes, and implantable sustained-release or biodegradable formulations. Such formulations can further comprise one or more additional ingredients including suspending, stabilizing, or dispersing agents. In one embodiment of a formulation for parenteral administration, the active ingredient is provided in dry (e.g., powder or granular) form for reconstitution with a suitable vehicle (e.g., sterile pyrogen-free water) prior to parenteral administration of the reconstituted composition. The pharmaceutical compositions can be prepared, packaged, or sold in the form of a sterile injectable aqueous or oily suspension or solution. This suspension or solution can be formulated according to the known art, and can comprise, in addition to the active ingredient, additional ingredients such as the dispersing agents, wetting agents, or suspending agents described herein. Such sterile injectable formulations can be prepared using a non-toxic parenterally-acceptable diluent or solvent, such as water or 1,3-butanediol, for example. Other acceptable diluents and solvents include Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation can comprise pharmaceutically acceptable polymeric or hydrophobic materials such as an emulsion, an ion exchange resin, a sparingly soluble polymer, or a sparingly soluble salt.

These compositions may also contain adjuvants such as preserving, wetting, emulsifying, and/or dispersing agents. Prevention of microorganism contamination of the compositions can be accomplished by the addition of various antibacterial and antifungal agents, for example, parabens, chlorobutanol, phenol, and sorbic acid. It may also be desirable to include isotonic agents, for example, sugars, and sodium chloride. Prolonged absorption of injectable pharmaceutical compositions can be brought about by the use of agents capable of delaying absorption, for example, aluminum monostearate and/or gelatin.

Dosage forms can include solid or injectable implants or depots. In certain embodiments, the implant comprises an aliquot of the particulate delivery system and a biodegradable polymer. In certain embodiments, a suitable biodegradable polymer can be selected from the group consisting of a polyaspartate, polyglutamate, poly(L-lactide), a poly(D,L-lactide), a poly(lactide-co-glycolide), a poly(ε-caprolactone), a polyanhydride, a poly(beta-hydroxy butyrate), a poly(ortho ester), and a polyphosphazene.

Solid dosage forms for oral administration include capsules, tablets, powders, and granules. In such solid dosage forms, the particulate delivery system is optionally admixed with at least one inert customary excipient (or carrier) such as sodium citrate or dicalcium phosphate or (a) fillers or extenders, as for example, starches, lactose, sucrose, mannitol, or silicic acid; (b) binders, as for example, carboxymethylcellulose, alginates, gelatin, polyvinylpyrrolidone, sucrose, or acacia; (c) humectants, as for example, glycerol; (d) disintegrating agents, as for example, agar-agar, calcium carbonate, potato or tapioca starch, alginic acid, certain complex silicates, or sodium carbonate; (c) solution retarders, as for example, paraffin; (f) absorption accelerators, as for example, quaternary ammonium compounds; (g) wetting agents, as for example, cetyl alcohol or glycerol monostearate; (h) adsorbents, as for example, kaolin or bentonite; and/or (i) lubricants, as for example, talc, calcium stearate, magnesium stearate, solid polyethylene glycols, sodium lauryl sulfate, or mixtures thereof. In the case of capsules and tablets, the dosage forms may also comprise buffering agents.

A tablet comprising the particulate delivery system can, for example, be made by compressing or molding the active ingredient, optionally with one or more additional ingredients. Compressed tablets can be prepared by compressing, in a suitable device, the active ingredient in a free-flowing form such as a powder or granular preparation, optionally mixed with one or more of a binder, a lubricant, an excipient, a surface-active agent, and a dispersing agent. Molded tablets can be made by molding, in a suitable device, a mixture of the active ingredient, a pharmaceutically acceptable carrier, and at least sufficient liquid to moisten the mixture. Pharmaceutically acceptable excipients used in the manufacture of tablets include inert diluents, granulating and disintegrating agents, binding agents, and lubricating agents. Known dispersing agents include potato starch and sodium starch glycolate. Known surface active agents include sodium lauryl sulfate. Known diluents include calcium carbonate, sodium carbonate, lactose, microcrystalline cellulose, calcium phosphate, calcium hydrogen phosphate, and sodium phosphate. Known granulating and disintegrating agents include corn starch and alginic acid. Known binding agents include gelatin, acacia, pre-gelatinized maize starch, polyvinylpyrrolidone, and hydroxypropyl methylcellulose. Known lubricating agents include magnesium stearate, stearic acid, silica, and talc.

Tablets can be non-coated or they can be coated using known methods to achieve delayed disintegration in the gastrointestinal tract of a human, thereby providing sustained release and absorption of the particulate delivery system, e.g. in the region of the Peyer's patches in the small intestine. By way of example, a material such as glyceryl monostearate or glyceryl distearate can be used to coat tablets. Further by way of example, tablets can be coated using methods described in U.S. Pat. Nos. 4,256,108; 4,160,452; and 4,265,874 to form osmotically-controlled release tablets. Tablets can further comprise a sweetening agent, a flavoring agent, a coloring agent, a preservative, or some combination of these in order to provide pharmaceutically elegant and palatable preparation.

Solid dosage forms such as tablets, dragees, capsules, and granules can be prepared with coatings or shells, such as enteric coatings and others well known in the art. They may also contain opacifying agents, and can also be of such composition that they release the particulate delivery system in a delayed manner. Examples of embedding compositions that can be used are polymeric substances and waxes. The active compounds can also be in micro-encapsulated form, if appropriate, with one or more of the above-mentioned excipients.

Solid compositions of a similar type may also be used as fillers in soft or hard filled gelatin capsules using such excipients as lactose or milk sugar, as well as high molecular weight polyethylene glycols. Hard capsules comprising the particulate delivery system can be made using a physiologically degradable composition, such as gelatin. Such hard capsules comprise the particulate delivery system, and can further comprise additional ingredients including, for example, an inert solid diluent such as calcium carbonate, calcium phosphate, or kaolin. Soft gelatin capsules comprising the particulate delivery system can be made using a physiologically degradable composition, such as gelatin. Such soft capsules comprise the particulate delivery system, which can be mixed with water or an oil medium such as peanut oil, liquid paraffin, or olive oil.

Oral compositions can be made, using known technology, which specifically release orally administered agents in the small or large intestines of a human patient. For example, formulations for delivery to the gastrointestinal system, including the colon, include enteric coated systems, based, e.g., on methacrylate copolymers such as poly(methacrylic acid, methyl methacrylate), which are only soluble at pH 6 and above, so that the polymer only begins to dissolve on entry into the small intestine. The site where such polymer formulations disintegrate is dependent on the rate of intestinal transit and the amount of polymer present. For example, a relatively thick polymer coating is used for delivery to the proximal colon (Hardy et al., 1987 Aliment. Pharmacol. Therap. 1:273-280). Polymers capable of providing site-specific colonic delivery can also be used, wherein the polymer relies on the bacterial flora of the large bowel to provide enzymatic degradation of the polymer coat and hence release of the drug. For example, azopolymers (U.S. Pat. No. 4,663,308), glycosides (Friend et al., 1984, J. Med. Chem. 27:261-268) and a variety of naturally available and modified polysaccharides (see PCT application PCT/GB89/00581) can be used in such formulations.

Pulsed release technology such as that described in U.S. Pat. No. 4,777,049 can also be used to administer the particulate delivery system to a specific location within the gastrointestinal tract. Such systems permit delivery at a predetermined time and can be used to deliver the particulate delivery system, optionally together with other additives that may alter the local microenvironment to promote stability and uptake, directly without relying on external conditions other than the presence of water to provide in vivo release.

Liquid dosage forms for oral administration include pharmaceutically acceptable emulsions, solutions, suspensions, syrups, and elixirs. In addition to the active compounds, the liquid dosage form may contain inert diluents commonly used in the art, such as water or other solvents, isotonic saline, solubilizing agents and emulsifiers, as for example, ethyl alcohol, isopropyl alcohol, ethyl carbonate, ethyl acetate, benzyl alcohol, benzyl benzoate, propylene glycol, 1,3-butylene glycol, dimethylformamide, oils, e.g., almond oil, arachis oil, coconut oil, cottonseed oil, groundnut oil, corn germ oil, olive oil, castor oil, sesame seed oil, MIGLYOL™, glycerol, fractionated vegetable oils, mineral oils such as liquid paraffin, tetrahydrofurfuryl alcohol, polyethylene glycols, fatty acid esters of sorbitan, or mixtures of these substances. Besides such inert diluents, the composition can also include adjuvants, such as wetting agents, emulsifying and suspending agents, demulcents, preservatives, buffers, salts, sweetening, flavoring, coloring and perfuming agents. Suspensions, in addition to the active compound, may contain suspending agents, as for example, ethoxylated isostearyl alcohols, polyoxyethylene sorbitol or sorbitan esters, microcrystalline cellulose, hydrogenated edible fats, sodium alginate, polyvinylpyrrolidone, gum tragacanth, gum acacia, agar-agar, and cellulose derivatives such as sodium carboxymethylcellulose, methylcellulose, hydroxypropylmethylcellulose, aluminum metahydroxide, bentonite, or mixtures of these substances. Liquid formulations of a pharmaceutical composition of the disclosure that are suitable for oral administration can be prepared, packaged, and sold either in liquid form or in the form of a dry product intended for reconstitution with water or another suitable vehicle prior to use.

Known dispersing or wetting agents include naturally-occurring phosphatides such as lecithin, condensation products of an alkylene oxide with a fatty acid, with a long chain aliphatic alcohol, with a partial ester derived from a fatty acid and a hexitol, or with a partial ester derived from a fatty acid and a hexitol anhydride (e.g. polyoxyethylene stearate, heptadecacthyleneoxycetanol, polyoxyethylene sorbitol monooleate, and polyoxyethylene sorbitan monooleate, respectively). Known emulsifying agents include lecithin and acacia. Known preservatives include methyl, ethyl, or n-propyl-para-hydroxybenzoates, ascorbic acid, and sorbic acid. Known sweetening agents include, for example, glycerol, propylene glycol, sorbitol, sucrose, and saccharin. Known thickening agents for oily suspensions include, for example, beeswax, hard paraffin, and cetyl alcohol.

For topical administration liquids, suspension, lotions, creams, gels, ointments, drops, suppositories, sprays and powders may be used. Conventional pharmaceutical carriers, aqueous, powder or oily bases, and thickeners can be used as necessary or desirable.

In other embodiments, the pharmaceutical composition can be prepared as a nutraceutical, i.e., in the form of, or added to, a food (e.g., a processed item intended for direct consumption) or a foodstuff (e.g., an edible ingredient intended for incorporation into a food prior to ingestion). Examples of suitable foods include candies such as lollipops, baked goods such as crackers, breads, cookies, and snack cakes, whole, pureed, or mashed fruits and vegetables, beverages, and processed meat products. Examples of suitable foodstuffs include milled grains and sugars, spices and other seasonings, and syrups. The particulate delivery systems described herein are not exposed to high cooking temperatures for extended periods of time, in order to minimize degradation of the compounds.

Compositions for rectal or vaginal administration can be prepared by mixing a particulate delivery system with suitable non-irritating excipients or carriers such as cocoa butter, polyethylene glycol or a suppository wax, which are solid at ordinary room temperature, but liquid at body temperature, and therefore, melt in the rectum or vaginal cavity and release the particulate delivery system. Such a composition can be in the form of, for example, a suppository, a retention enema preparation, and a solution for rectal or colonic irrigation. Suppository formulations can further comprise various additional ingredients including antioxidants and preservatives. Retention enema preparations or solutions for rectal or colonic irrigation can be made by combining the active ingredient with a pharmaceutically acceptable liquid carrier. As is known in the art, enema preparations can be administered using, and can be packaged within, a delivery device adapted to the rectal anatomy of a human. Enema preparations can further comprise various additional ingredients including antioxidants and preservatives.

A pharmaceutical composition of the disclosure can be prepared, packaged, or sold in a formulation suitable for pulmonary administration via the buccal cavity. Such compositions are conveniently in the form of dry powders for administration using a device comprising a dry powder reservoir to which a stream of propellant can be directed to disperse the powder or using a self-propelling solvent/powder-dispensing container such as a device comprising the particulate delivery system suspended in a low-boiling propellant in a sealed container. Dry powder compositions may include a solid fine powder diluent such as sugar and are conveniently provided in a unit dose form. Low boiling propellants generally include liquid propellants having a boiling point below 65 degrees F. at atmospheric pressure. Generally, the propellant can constitute 50 to 99.9% (w/w) of the composition, and the active ingredient can constitute 0.1 to 20% (w/w) of the composition. The propellant can further comprise additional ingredients such as a liquid non-ionic or solid anionic surfactant or a solid diluent, e.g., having a particle size of the same order as particles comprising the particulate delivery system.

Pharmaceutical compositions of the disclosure formulated for pulmonary delivery can also provide the active ingredient in the form of droplets of a suspension. Such formulations can be prepared, packaged, or sold as aqueous or dilute alcoholic suspensions, optionally sterile, comprising the particulate delivery system, and can conveniently be administered using any nebulization or atomization device. Such formulations can further comprise one or more additional ingredients including a flavoring agent such as saccharin sodium, a volatile oil, a buffering agent, a surface-active agent, or a preservative such as methylhydroxybenzoate.

The formulations described herein as being useful for pulmonary delivery are also useful for intranasal delivery of a pharmaceutical composition of the disclosure. Another formulation suitable for intranasal administration is a coarse powder comprising the particulate delivery system. Such a formulation is administered in the manner in which snuff is taken, i.e., by rapid inhalation through the nasal passage from a container of the powder held close to the nares.

A pharmaceutical composition of the disclosure can be prepared, packaged, or sold in a formulation suitable for buccal administration. Such formulations can, for example, be in the form of tablets or lozenges made using conventional methods, and can, for example, comprise 0.1 to 20% (w/w) particulate delivery system, the balance comprising an orally dissolvable or degradable composition and, optionally, one or more of the additional ingredients described herein. Alternately, formulations suitable for buccal administration can comprise a powder or an aerosolized or atomized solution or suspension comprising the particulate delivery system.

Reference will now be made to specific examples illustrating the disclosure. It is to be understood that the examples are provided to illustrate exemplary embodiments and that no limitation to the scope of the disclosure is intended thereby.

### EXAMPLES

### Example 1: Production of Yeast Particles

YPs are typically 3-5 μm hollow and porous microparticles derived from Baker's yeast that are composed primarily of ˜80% 1→6-β branched, 1→3-β-glucan, 2-4% chitin and 40% mannan w/w. Yeast particles are readily available, biodegradable, substantially spherical particles about 2-4 μm in diameter.

Methods of preparing extracted yeast cell wall particles are known in the art, and are described, for example, in U.S. Pat. Nos. 4,992,540, 5,082,936, 5,028,703, 5,032,401, 5,322,841, 5,401,727, 5,504,079, 5,968,811, 6,444,448 B1, 6,476,003 B1, published U.S. applications 2003/0216346 A1, 2004/0014715 A1, and published PCT application WO 02/12348 A2, the disclosures of which are incorporated herein by reference.

A form of extracted yeast cell wall particles, referred to as “whole glucan particles” or “WPGs” (See U.S. Pat. Nos. 5,032,401 and 5,607,677) may be modified to facilitate improved retention and/or delivery of payload molecules. Such improvements feature trapping molecules and nanoparticles as well as pluralities of said trapping molecules and nanoparticles, formulated in specific forms to achieve the desired improved delivery properties. As used herein, a WGP is typically a whole glucan particle of >90% beta glucan purity.

### Preparation of Glucan Particles (GPs)

Glucan particles (GPS), also referred to herein as yeast glucan particles (“YGPs”), are a purified hollow yeast cell ‘ghost’ containing a rich β-glucan sphere, generally 2-4 microns in diameter. In general, glucan particles can be prepared from yeast cells by the extraction and purification of the alkali-insoluble glucan fraction from the yeast cell walls. The yeast cells can be treated with an aqueous hydroxide solution without disrupting the yeast cell walls, which digests the protein and intracellular portion of the cell, leaving the glucan wall component devoid of significant protein contamination, and having substantially the unaltered cell wall structure of β(1-6) and β(1-3) linked glucans. The 1,3-β-glucan outer shell provides for receptor-mediated uptake by phagocytic cells, e.g., macrophages, expressing β-glucan receptors.

Glucan particles can be made as follows. Yeast particles (S. cerevisiae), Biorigin MOS55, are suspended in 1 liter of 1M NaOH and heated to 85° C. The cell suspension is stirred vigorously for 1 hour at this temperature. The insoluble material containing the cell walls is recovered by centrifuging. This material is then suspended in 1M NaOH, heated, and stirred vigorously for 1 hour. The suspension is allowed to cool to room temperature and the extraction is continued for a further 16 hours. The insoluble residue is recovered by centrifugation. This material is finally extracted in water brought to pH 4.5 with HCl. The insoluble residue is recovered by centrifugation and washed three times with water, isopropanol, and acetone. The resulting slurry is placed in glass trays and dried under reduced pressure to produce a fine white powder.

### Preparation of Glucan Lipid Particles (GLPs)

GLPs retain some of the yeast cellular lipid content, which creates a more hydrophobic inner cavity ideal for loading of hydrophobic payloads. GLPs are prepared by modifying the method of preparation of GPs described above. For preparation of GLPs, washing with isopropanol and acetone is eliminated and instead the insoluble residue recovered by centrifugation is washed three times with water. The particles are dried by lyophilization or spray drying.

### Commercial Yeast Particles (YPs)

Yeast particles (YPs) were purchased from Biorigin (Louisville, KY, USA) or Lesaffre (Marcq-en-Baroeul, France). These YPs contained sufficient amounts of lipids to provide for a hydrophobic reservoir that attracts hydrophobic payloads to diffuse into the center of the particle accomplishing loading.

### Whole Glucan Particles (WGPs)

A more detailed description of processes for preparing WPGs can be found in U.S. Pat. Nos. 4,810,646, 4,992,540, 5,028,703, 5,607,677, and 5,741,495 (incorporated herein by reference). For example, U.S. Pat. No. 5,028,703 discloses that yeast WGP particles can be produced from yeast strain R4 cells in fermentation culture. The cells are harvested by batch centrifugation at 8000 rpm for 20 minutes in a Sorval RC2-B centrifuge. The cells are washed twice in distilled water in order to prepare them for the extraction of the whole glucan. The first step involved resuspending the cell mass in 1 liter 4% w/v NaOH and heating to 100° C. The cell suspension is stirred vigorously for 1 hour at this temperature. The insoluble material containing the cell walls is recovered by centrifuging at 2000 rpm for 15 minutes. This material is suspended in 2 liters, 3% w/v NaOH and heated to 75° C. The suspension is stirred vigorously for 3 hours at this temperature. The suspension is then allowed to cool to room temperature and the extraction can be continued for a further 16 hours. The insoluble residue is recovered by centrifugation at 2000 rpm for 15 minutes. This material is finally extracted in 2 liters, 3% w/v NaOH brought to pH 4.5 with HCl, at 75° C. for 1 hour. The insoluble residue is recovered by centrifugation and washed three times with 200 milliliters water, once with 200 milliliters dehydrated ethanol, and twice with 200 milliliters dehydrated ethyl ether. The resulting slurry is placed on petri plates and dried.

Varying degrees of purity of glucan particles are achieved by modifying the extraction/purification process. In general, these GPs are on the order of 80-85% pure on a w/w basis of beta glucan and, following the introduction of payload, trapping, or other components, become of a slightly lesser “purity.” In exemplary embodiments, GPs are <90% beta glucan purity.

### Preparation of YCP Particles

Yeast cells (Rhodotorula sp.) derived from cultures obtained from the American Type Culture Collection (ATCC, Manassas, Va.) are acrobically grown to stationary phase in YPD at 30° C. Rhodotorula sp. cultures available from ATCC include Nos. 886, 917, 9336, 18101, 20254, 20837 and 28983. Cells are harvested by batch centrifugation at 2000 rpm for 10 minutes. The cells are then washed once in distilled water and then re-suspended in water brought to pH 4.5 with HCl, at 75° C. for 1 hour. The insoluble material containing the cell walls is recovered by centrifuging. This material is then suspended in 1 liter, 1M NaOH and heated to 90° C. for 1 hour. The suspension is allowed to cool to room temperature and the extraction is continued for a further 16 hours. The insoluble residue is recovered by centrifugation and washed twice with water, isopropanol, and acetone. The resulting slurry is placed in glass trays and dried at room temperature to produce 2.7 g of a fine light brown powder.

In alternative embodiments, YGPs, e.g., activated YGPs, are grafted with chitosan on the surface, for example, to increase total surface chitosan. Chitosan can further be acetylated to form chitin (YGCP), in certain embodiments. Such particles have equivalent properties in vivo when detected by the immune system of a subject or patient.

### Preparation of YGMP Particles

S. cerevisiae (100 g Fleishman's Baker's yeast) was suspended in 1 liter 1M NaOH and heated to 55° C. The cell suspension was mixed for 1 hour at this temperature. The insoluble material containing the cell walls was recovered by centrifuging at 2000 rpm for 10 minutes. This material was then suspended in 1 liter of water and brought to pH 4-5 with HCl, and incubated at 55° C. for 1 hour. The insoluble residue was recovered by centrifugation and washed once with 1000 milliliters water, four times with 200 milliliters dehydrated isopropanol and twice with 200 milliliters acetone. The resulting slurry was placed in a glass tray and dried at room temperature to produce 12.4 g of a fine, slightly off-white powder.

S. cerevisiae (75 g SAF-Mannan) was suspended in 1 liter water and adjusted to pH 12-12.5 with 1M NaOH and heated to 55° C. The cell suspension was mixed for 1 hour at this temperature. The insoluble material containing the cell walls was recovered by centrifuging at 2000 rpm for 10 minutes. This material was then suspended in 1 liter of water and brought to pH 4-5 with HCl, and incubated at 55° C. for 1 hour. The insoluble residue was recovered by centrifugation and washed once with water, dehydrated isopropanol, and acetone. The resulting slurry was placed in a glass tray and dried at room temperature to produce 15.6 g of a fine slightly off-white powder.

### Preparation of Glucan Mannan Lipid Particles (GMLPs)

GMLPs were prepared by the procedure described above for preparation of YGLPs but without the steps requiring washing with isopropanol and acetone.

### Example 2: Ensilication of Payload in Glucan Particles

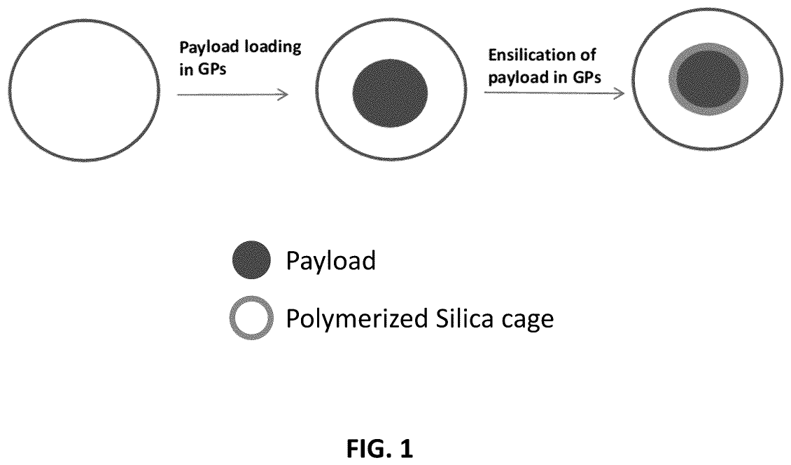

FIG. 1 shows the schematic diagram for ensilicating payloads inside YPs.

The chemical reactions resulting in polymerization and hydrolysis of tetraethylorthosilicate (TEOS) are described by Buckley et al. (Buckley, A. M., & Greenblatt, M. (1994). The sol-gel preparation of silica gels. Journal of Chemical Education, 71 (7), 599-602).

Procedure for ensilication of payloads with GPs was devised by modifying the procedure described by Chen et al. (Chen, Y-C; et al. Thermal stability, storage and release of proteins with tailored fit in silica. Scientific Reports, 2017, 7, 46568). Partially pre-polymerized TEOS of a particular size capable of diffusing into hydrated YPs was used. The TEOS could dissolve the payload and polymerize to trap the payload in a silicate glass to provide increased stability. The speed of the ensilication reaction was decreased and the reaction was carried out in half hydrodynamic volume. These modifications ensured that the ensilicated payload remained inside the YPs. In this procedure no polymerization occurred outside the particles. As polymerization outside the YPs leadings to particle clumping, the instant modified procedure ensured that YPs remained as individual particles without aggregating. The TEOS polymerization on the interior of YPs ensured that payload trapping/ensilication occurred inside the YPs instead of on the outside of YPs.

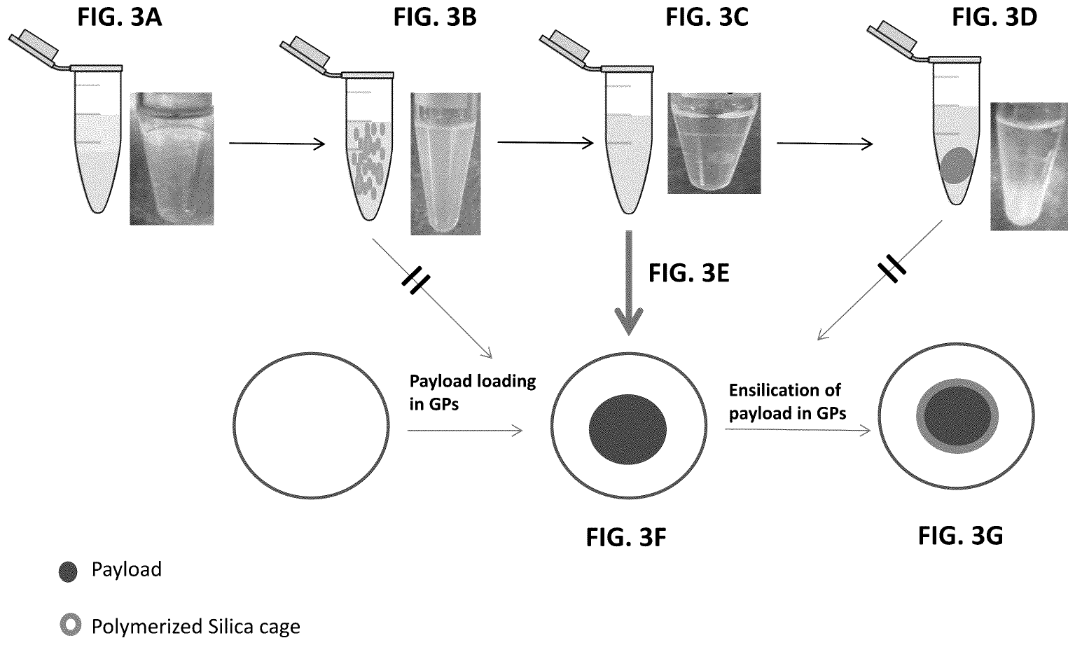

Ensilication procedure of payloads within GPs involves several steps which are shown in FIG. 3A-G. Initial TEOS (top phase, d=0.93 g/mL) and aqueous (acid or base) solution (bottom phase) (FIG. 3A) when mixed forms a TEOS-aqueous emulsion composed of partially pre-polymerized TEOS far from gel point (FIG. 3B). Upon further incubation TEOS-aqueous solution forms partially pre-polymerized TEOS close to gel point (FIG. 3C) which upon further incubation forms silica gel as TEOS fully polymerizes (FIG. 3D). To ensilicate payloads in GPs, TEOS-aqueous solution close to gel point is loaded into GPs containing payloads. The TEOS then full polymerizes inside GPs thereby forming a silica cage around the payloads within GPs (FIG. 3G).

### Example 3: Ensilication of Bovine Serum Albumin (BSA, MW=66.5 kD) within GPs

The efficiency of ensilication of BSA within GPs was tested. 20 μL of a 25 mg rhodamine labeled BSA (rBSA)/mL aqueous solution was added to a dry sample of 5 mg fluorescent GPs (fGPs). The samples were mixed, incubated at room temperature for 30 minutes, frozen and lyophilized to remove solvent. The loading cycle was repeated by adding 10 μL of water to the dry fGP rBSA sample, the sample was mixed and lyophilized to remove solvent.

TEOS was mixed with 0.01 M HCl solution (pH 2). The mixture was mixed for 1 hour to achieve TEOS pre-polymerization and formation of a single aqueous phase in which the TEOS was close to gel point. Solutions with different ratios of TEOS and 0.01 M HCl were prepared to target TEOS:rBSA weight ratios from 0 to 24.9. The partially pre-polymerized TEOS mixture was added to the dry fGP-rBSA pellet (20 μL/5 mg fGP). The sample was mixed and incubated at room temperature for 1 hour.

The ensilicated fGP-rBSA samples were washed three times with 0.9% saline. The ensilicated fGP rBSA pellets were evaluated by microscopy to assess efficient trapping of rBSA within the cavity of fGPs, and percent protein encapsulation was quantified by SDS-PAGE. Encapsulation of rBSA within fGPs was visualized by brightfield and fluorescent microscopy overlays of the fGP rBSA [SiO₂]ₙ formulation.

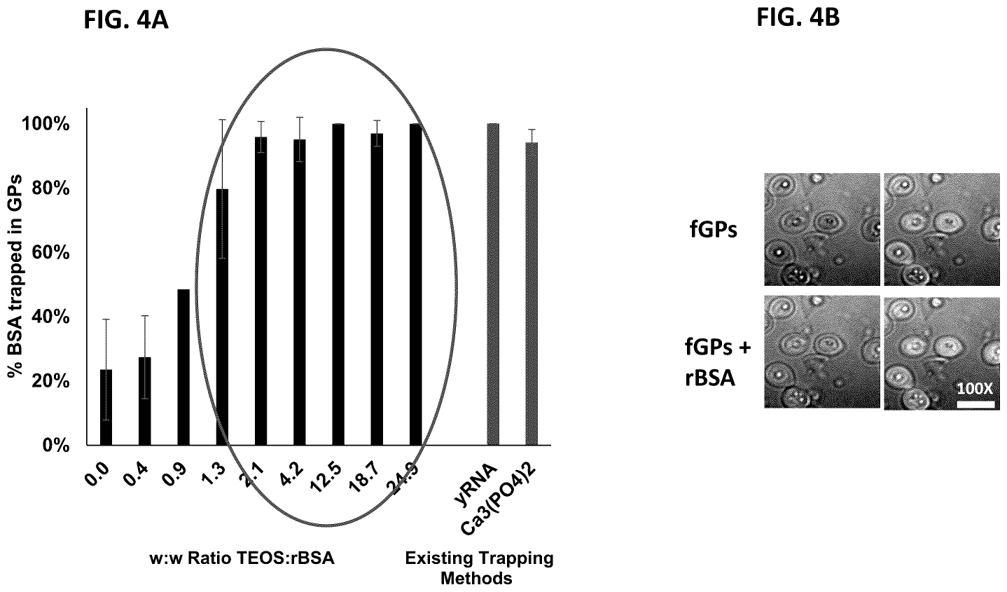

TEOS partially pre-polymerized at pH 2 and used at TEOS:rBSA ratios of 1.3 to 24.9 ensilicated and trapped rBSA within GPs at high efficiency (>80%) comparable to conventional methods of trapping payload within GPs using yRNA or Ca₃(PO₄)₂ (FIG. 4A). Fluorescent micrography confirmed that the rBSA was encapsulated within GPs (FIG. 4B).

Ensilication of rBSA in GPs Using a Fluorescent Silica Shell (TEOS/f-APTES)

Fluorescent precursor for ensilication (f-APTES) was synthesized by reaction of 3-amino-propyl-triethoxysilane with fluorescein-isothiocyanate (FITC) in ethanol at room temperature under nitrogen atmosphere. A mixture of 0.01% f-APTES and 99.99% TEOS was used for ensilication.

Fluorescent rBSA was loaded in GPs as described above. GPs used were non-fluorescent. The fAPTES/TEOS ensilication mixture was prepared in 0.01 M HCl as described above. Encapsulation of rBSA within GPs was visualized by brightfield and fluorescent microscopy overlays.

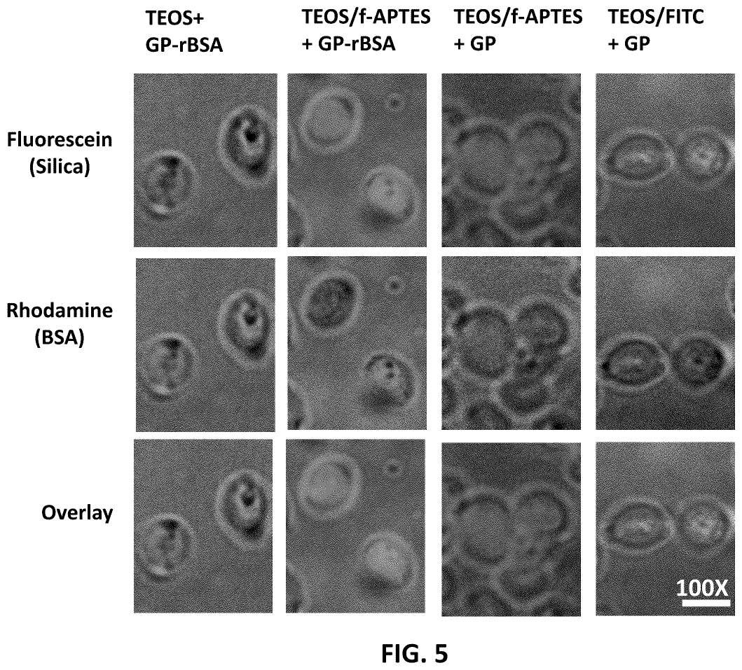

FIG. 5 shows brightfield and fluorescent microscopy overlays of GPs with or without rBSA and fluorescent silica shell (TEOS/f-APTES) and confirms that fully polymerized silica shells were located inside GPs.

### Example 4: Interaction of Ensilicated fGP-BSA Samples with Macrophages

Macrophages Efficiently Uptake fGP BSA [SiO₂]ₙ Via Phagocytosis

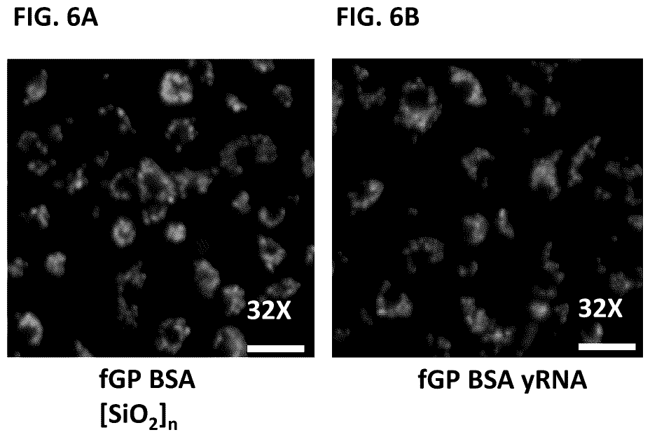

Sterile fGP samples in 0.9% saline were diluted at a concentration of 1×10⁸ particles/mL. 10 μL of fGP samples (1×10⁶ particles) were added to wells in a 96-well plate containing ˜1×10⁵ B6 macrophage cells per well (fGP:cell ratio of 10:1) in complete Dulbecco's Modified Eagle Medium (DMEM). The plates were incubated at 37° C., 5% CO₂ for 24 hours and then particle uptake was assessed by tracking of fGPs by fluorescent microscopy. FIG. 6 shows that fGPs containing ensilicated BSA were phagocytosed with equal efficiency as the control fGPs containing BSA trapped via conventional methods using yRNA.

### Ensilicated GP-BSA Samples are not Toxic to Macrophages

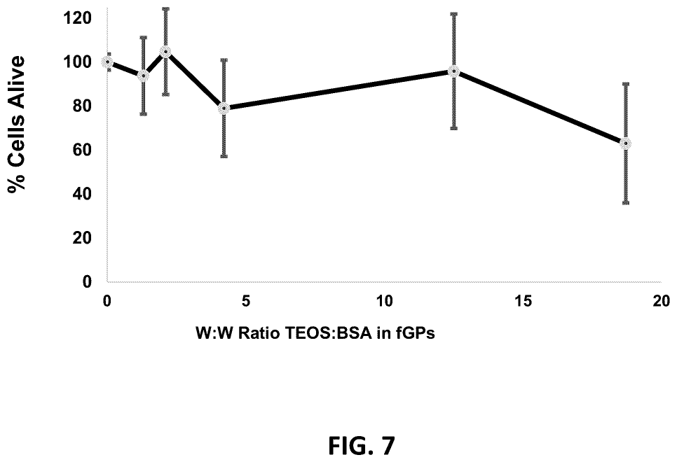

Sterile ensilicated fGP BSA samples in 0.9% saline were added to wells in a 96-well plate containing B6 macrophage cells. The fGPs were added at a ratio of 10:1 particles:cell. The plates were incubated at 37° C., 5% CO₂ for 24 hours. ALAMAR BLUE™ solution was added (10 μL per well) and the plate was incubated for 30 minutes at 37° C., 5% CO₂. ALAMAR BLUE™ fluorescence was measured (excitation wavelength =530 nm, emission wavelength=590 nm). Fluorescence response is dependent on the reduction of the ALAMAR BLUE™ indicator by metabolically active cells and thus, is a measure of cell number and viability. The percent of live cells was calculated from the fluorescence response of the sample relative to the response of control wells containing buffer (PBS) or empty GPs. FIG. 7 shows that at low W:W ratio of TEOS:BSA, fGPs exhibited very little toxicity on the macrophage cell line. Even at higher TEOS:BSA W:W ratios, more than 60% of the cells were viable after exposure to fGPs containing ensilicated BSA. The results show that GPs containing ensilicated payloads were not cytotoxic to macrophages.

### Ensilicated BSA is Efficiently Released Inside B6 Macrophage Cells

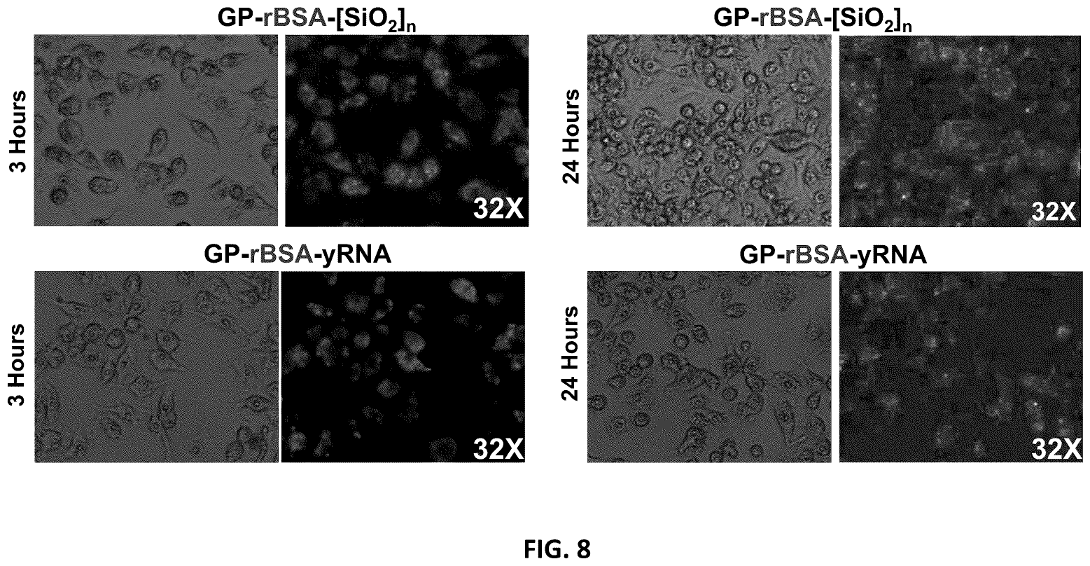

Following uptake of GPs containing ensilicated rBSA, release of rBSA was tracked as described above using rhodamine labeled BSA to track the location of the protein at the 3 hour and 24 hour time points following GP uptake by B6 cells. GPs encapsulating BSA trapped with yRNA were used as control. FIG. 8 shows that both ensilicated BSA and BSA trapped with yRNA were located within GPs at the 3 hour time point but were released within macrophage cells at the 24 hour time point. This result confirmed that ensilicated payload can be efficiently released inside macrophage cells.

### Example 5: Optimization of Ensilication of Bovine Serum Albumin (BSA, MW=66.5 kD)

### Effect of pH on Ensilication Efficiency

TEOS was partially pre-polymerized as described before. Solutions of different concentrations of HCl and NaOH were used to generate different pH conditions. TEOS was allowed to pre-polymerize for 1, 6 or 20 hours prior to loading it into GPs containing BSA payload.

Acid catalyzed TEOS forms long, linear polymers that easily entangle and gel. Base catalyzed TEOS forms short, branch clusters that may not trap payload with high efficiency. (Buckley et al., J. Chem. Ed. 1994). Percent of BSA trapped in GPs was measured as described before.

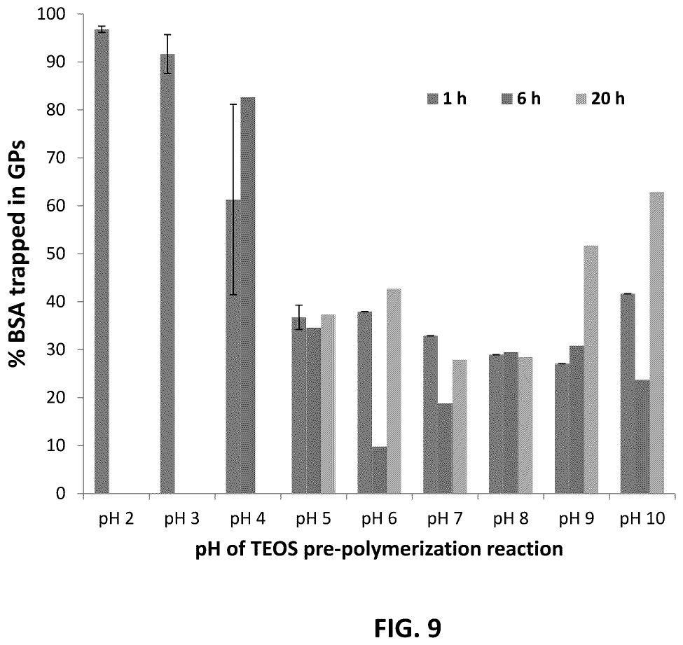

FIG. 9 shows the BSA trapping efficiency of TEOS partially pre-polymerized at various pH values for varying amounts of time prior to loading it into GPs containing BSA payload. Results show that 1 hour pre-polymerization time at pH 2 or pH3 is the most optimal condition for achieving highest BSA trapping efficiency. At pH of 4, a high efficiency could be achieved by increasing the pre-polymerization time to 6 hours. At pH of 9 or 10, a moderate trapping efficiency (50-70%) could be achieved by increasing pre-polymerization time to 20 hours. At pH values of 5-8, a very slow hydrolysis/condensation of TEOS occurred which reduced BSA trapping efficiency to below 40% at all pre-polymerization time points used.

Ensilication of Buffered GP-BSA with TEOS Partially Pre-Polymerized at pH2

The effect of the pH of GP-BSA and TEOS mixture on ensilication efficiency was tested. TEOS mixture was partially pre-polymerized at a pH 2 and mixed with GP-BSA to achieve a TEOS:BSA w/w ratio of 12.5:1. The various experimental conditions tested are summarized in Table 1.

TABLE 1
Experimental conditions used for testing ensilication
efficiency of TEOS partially pre-polymerized at
pH 2 with GP-BSA prepared in water or buffer.

| | | Post-loading | | |
|---|---|---|---|---|
| | | Resuspension | TEOS | |
| Experimental | GP-BSA | of GP-BSA in | Mixture at | Mixing |
| conditions | loading in | a buffer at pH | pH | Procedure |
| A (pH 2) | water | — | pH 2 | Resuspend |
| | | | | GP-BSA in |
| | | | | TEOS mixture |
| B (pH 2/pH 7 | Water | pH 7 | pH 2 | Add TEOS |
| Sequential | | | | mixture to |
| load) | | | | GP-BSA |
| C (pH 2/pH 7 | PBS* pH 7 | — | pH 2 | Resuspend |
| co-load) | | | | GP-BSA in |
| | | | | TEOS mixture |
| D (pH 7) | PBS pH 7 | — | PBS pH 7 | Resuspend |
| | | | | GP-BSA in |
| | | | | TEOS mixture |

*PBS = Phosphate buffered saline

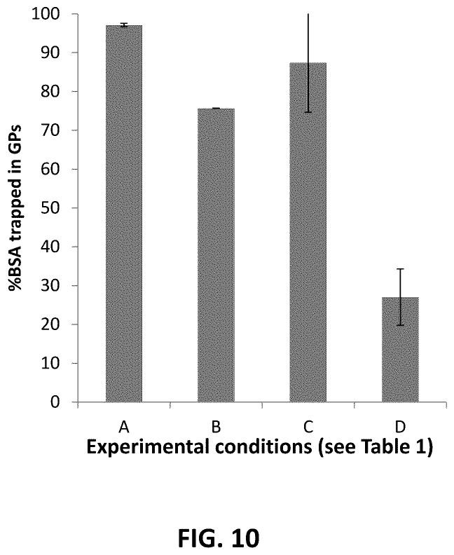

FIG. 10 shows that ensilication efficiency was the highest when GP-BSA was prepared in water and resuspended in TEOS mixture partially pre-polymerized at pH 2.

### Effect of Loading of BSA in GPs in the Presence of 6M Urea on Ensilication Efficiency

To test whether proteins could be loaded in GPs in the presence of denaturing conditions, BSA was loaded into GPs in the presence of water or in high concentration of urea (6M). Ensilication reaction was carried out as described before using TEOS partially pre-polymerized at pH 2. The w/w ratio of TEOS:BSA used was 12.5:1.

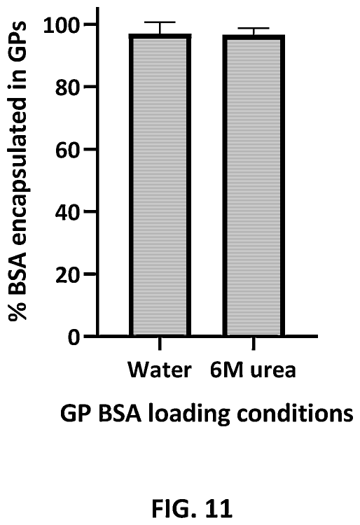

FIG. 11 shows that ensilication efficiency with BSA was unaffected in the presence of 6M urea.

### Effect of Loading of BSA in GPs Under High Salt Concentration on Ensilication Efficiency

To test whether proteins could be loaded in GPs under high salt concentrations, BSA was loaded into GPs in the presence of water or sodium chloride (NaCl) at a concentration of 1M or 5M. Ensilication reaction was carried out as described before using TEOS partially pre-polymerized at pH 2. The w/w ratio of TEOS:BSA used was 12.5:1.

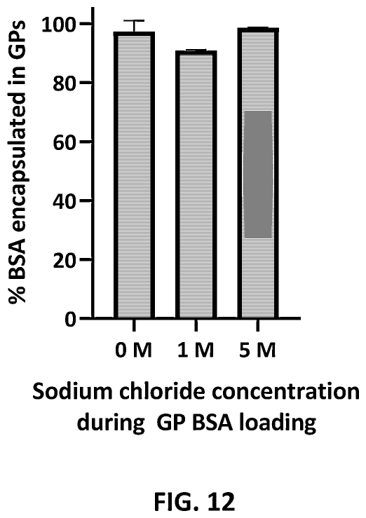

FIG. 12 shows that ensilication efficacy was not impacted when BSA was loaded in GPs in the presence of high concentrations of sodium chloride.

### Effect of Strong or Weak Acids and Incubation Conditions on Ensilication Efficiency

BSA was loaded in GPs as described before. TEOS was partially pre-polymerized as described before but at pH 4 using a strong (HCl) or a weak (acetic) acid. After mixing the partially pre-polymerized TEOS with GP-BSA, the mixture was allowed to incubate for 1, 4 or 18 hours at a temperature of 4° C. or 23° C. Percent of BSA trapped in GPs was calculated as described before.

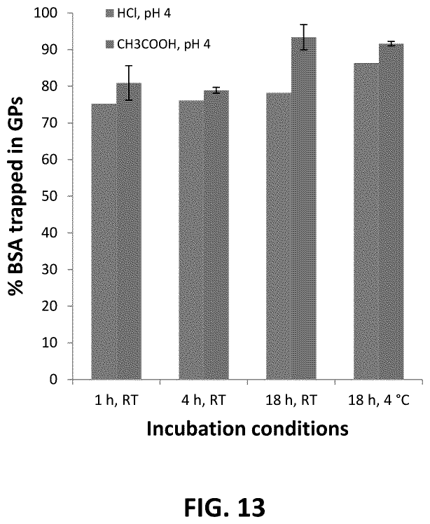

FIG. 13 shows that ensilication efficiency was high when TEOS was partially pre-polymerized in a weak acid and the TEOS-GP-BSA mixture was incubated at low temperatures for prolonged periods. These results indicate that proteins unstable at room temperature or in strong (pH 2) acids can still be efficiency encapsulated and ensilicated within GPs using alternative methods.

### Example 5: Encapsulation of Two Different Payloads in GPs

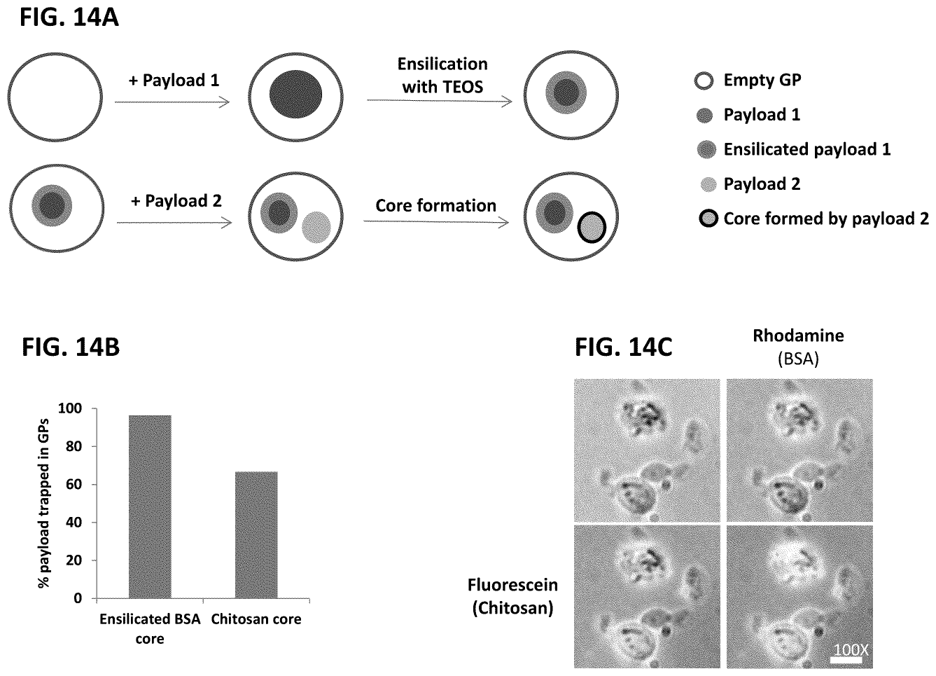

To test whether another payload could be added to GPs after ensilication of the first payload, chitosan was added to GPs after the GPs were loaded with BSA. FIG. 14A shows the schematic of the procedure to load two payloads in GPs. GPs were loaded with rhodamine labeled BSA (rBSA) as described before. GPs loaded with rBSA were then subjected to the ensilication protocol as described before. After ensilication of rBSA, fluorescent labeled chitosan (f-chitosan) was loaded in half hydrodynamic volume (5 μL/mg GP) using a solution of f-chitosan at pH 5, lyophilized and then phosphate buffer (pH 7) was added to precipitate the f-chitosan core.

FIG. 14B shows that both BSA and chitosan could be efficiently trapped within GPs following the sequential loading procedure shown in FIG. 14A. FIG. 14C shows that both BSA and chitosan payloads were trapped within GPs. This data confirms after encapsulation of one payload via ensilication, a second payload could be encapsulated within GPs.

### Example 6: Ensilication with Tetraethylorthogermanate (TEOG)

The ensilication efficiency of tetraethylorthogermanate (TEOG) was compared with TEOS. GPs were loaded with BSA as described above. TEOG was partially pre-polymerized by the same process as described before for TEOS at pH 2 and pH 10. GP-BSA was incubated with partially pre-polymerized TEOG to promote ensilication of BSA within GPs. TEOS was used as control for comparison of trapping efficiency.

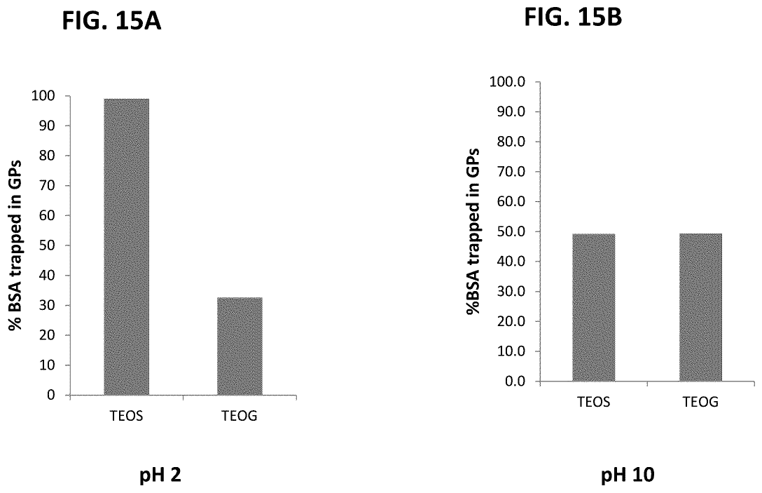

FIGS. 15A and B show the BSA trapping efficiencies of TEOG and TEOS at pH 2 and pH 10, respectively. At pH 2, TEOG gels rapidly, i.e., in less than 1 minute, thus preventing efficient loading of TEOG in GPs. However, at pH 10 TEOG gels more slowly over 10-20 minutes. Thus, the best trapping efficiency could be achieved with TEOG at pH 10.

### Example 7: Ensilicated Cores for Layer-by-Layer (LbL) Payload Absorption

### Layer-by-Layer Adsorption of Charged Polymers on GP Ensilicated Cores

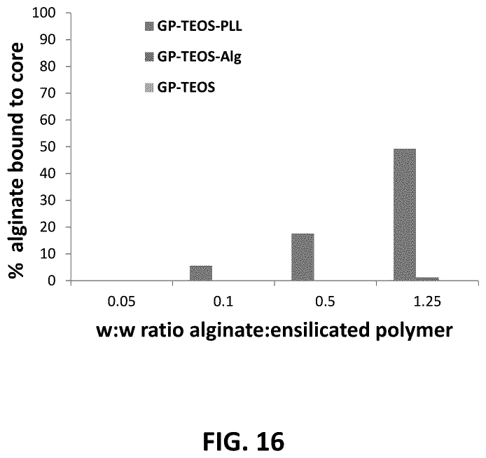

The ability of anionic alginate to adsorb on to GP partially ensilicated PLL or alginate cores was tested. Anionic fluorescent alginate was used. Data presented in FIG. 16 shows that anionic fluorescent alginate bound only to a GP cationic ensilicated PLL core. The percent alginate bound to the core increased as the w/w ratio of alginate:ensilicated polymer increased.

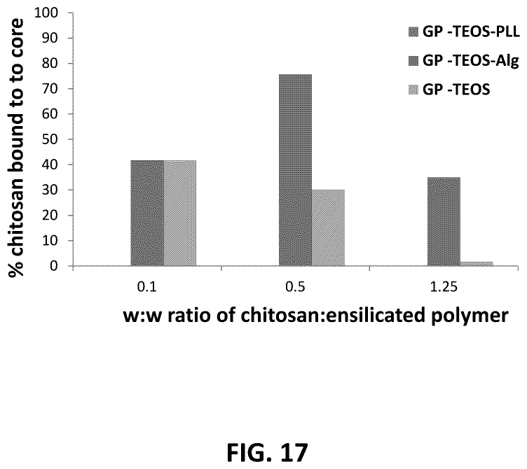

The ability of cationic fluorescent chitosan (MW 15 kD) to adsorb on to partially ensilicated GP PLL or alginate ensilicated cores were tested. Data presented in FIG. 17 shows that cationic fluorescent chitosan bound more efficiently to just GP TEOS or GP anionic alginate cores partially ensilicated by TEOS.

### Example 8: Stability of BSA Ensilicated within GPs

Ability of ensilication process to protect BSA from thermal denaturation and enzymatic digestion was tested.

### Stability of BSA in the Presence of Proteases

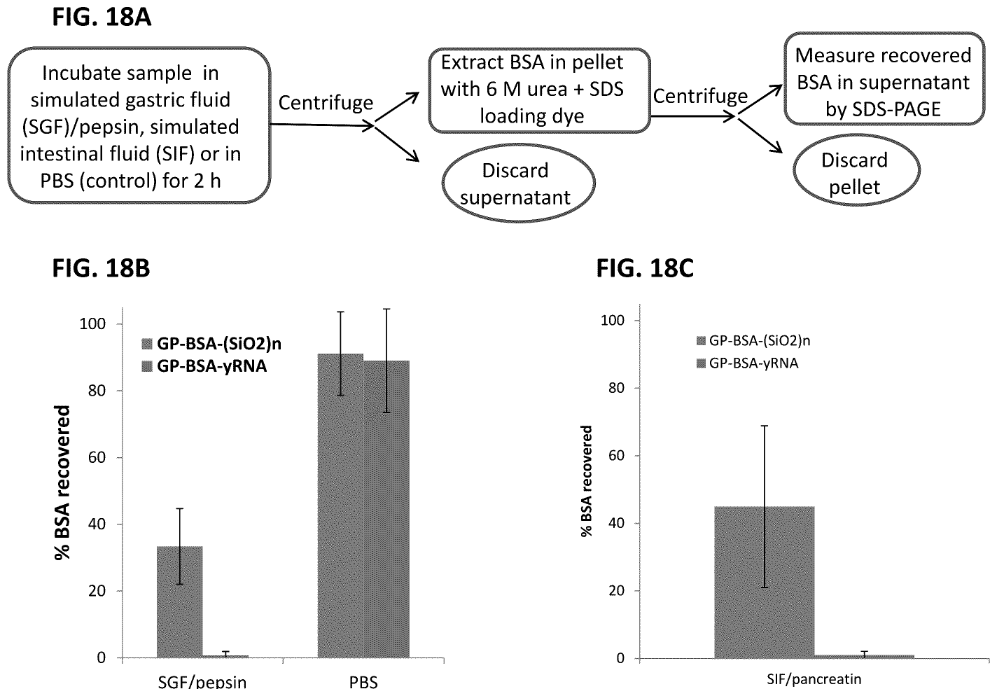

Samples of GPs containing ensilicated BSA were incubated with simulated gastric fluid (SGF), pepsin, simulated intestinal fluid (SIF) or pancreatin for 2 hours. Sample was centrifuged and supernatant was discarded. The BSA in the pellet was extracted with 6M Urca and SDS PAGE loading dye. The extracted BSA was centrifuged. The precipitated protein in the pellet was discarded and BSA remaining in the supernatant was quantified by SDS-PAGE. FIG. 18A shows a flowchart summarizing the steps for extraction and quantification of BSA after incubation of GP containing ensilicated BSA with SGF/pepsin.

Ensilicated BSA was more resistant to enzymatic digestion compared to BSA encapsulated via yRNA trapping when exposed to SGF/pepsin (FIG. 18B) or SIF/pancreatin (FIG. 18C).

Improved Stability of Ensilicated GP-BSA with Eudragit® Coating

EUDRAGIT® L100 is a commonly used polymethacrylate-based coating polymer available from Evonik Industries. Eudragit® L100 coats are water insoluble at pH<5.5 but dissolve at pH above 5.5. Eudragit® coating layer is useful in preventing drug release and degradation in the stomach.

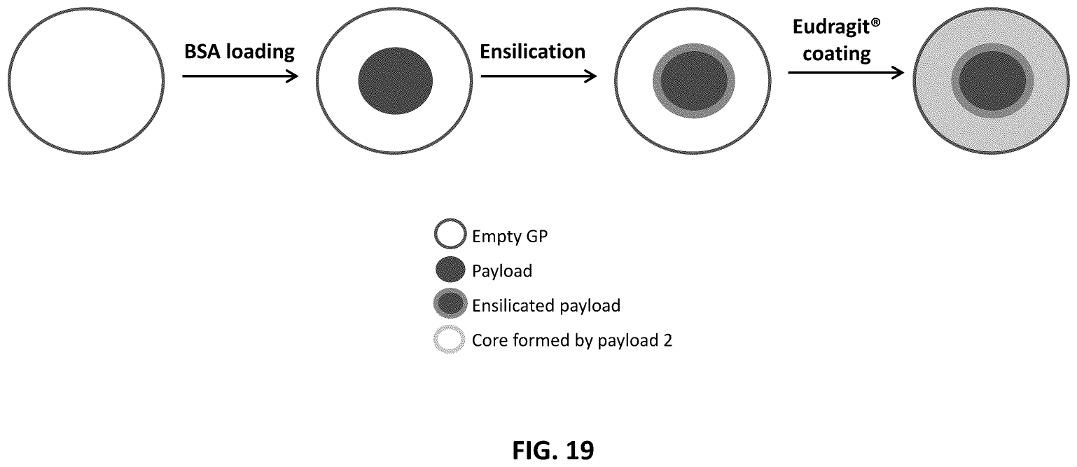

The ability of EUDRAGIT® coat to further enhance the stability of ensilicated BSA was tested. GPs containing ensilicated BSA were coated with EUDRAGIT® FIG. 19 shows the schematic of the procedure for preparation of EUDRAGIT® coated ensilicated payload within GPs.

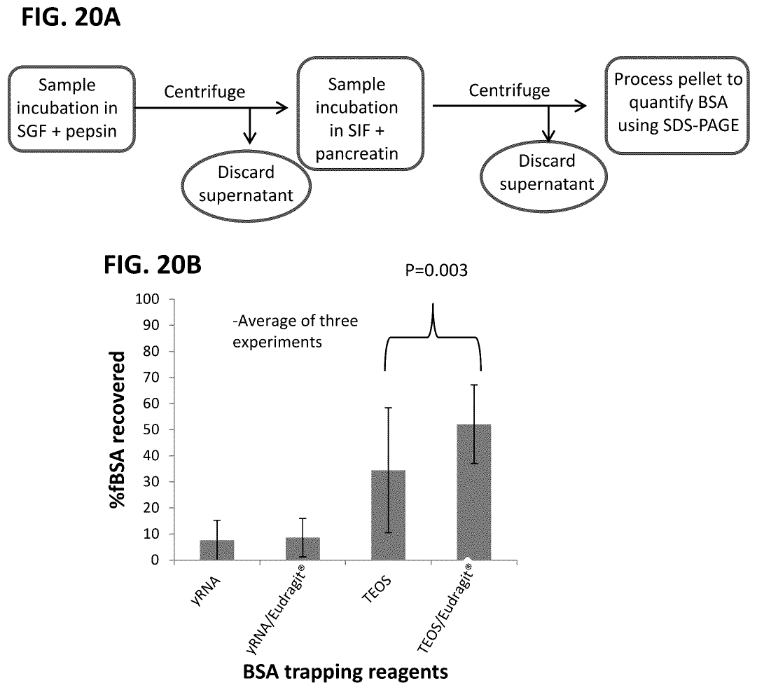

Samples of GPs containing ensilicated BSA coated with EUDRAGIT® were incubated with SGF/pepsin for 2 hours. Samples were centrifuged and supernatants discarded. Samples were further incubated with SIF/pancreatin for 2 hours. Samples were centrifuged and supernatants discarded. The BSA in the pellet was extracted with 6M urea and SDS loading dye. The extracted BSA was centrifuged. The precipitated protein in the pellet was discarded and BSA remaining in the supernatant was quantified by SDS-PAGE. FIG. 20A shows a flowchart summarizing the steps for extraction and quantification of BSA after incubation of GP containing EUDRAGIT® coated ensilicated BSA with SGF/pepsin followed by SIF/pancreatin. FIG. 20B shows percent of BSA remaining in GPs after exposure to enzymatic digestion. Data shown is the average from three different experiments.

Results confirm that in EUDRAGIT® L100 coated samples, BSA was more resistant to enzymatic digestion.

### Thermal Stability of BSA Ensilicated in GPs

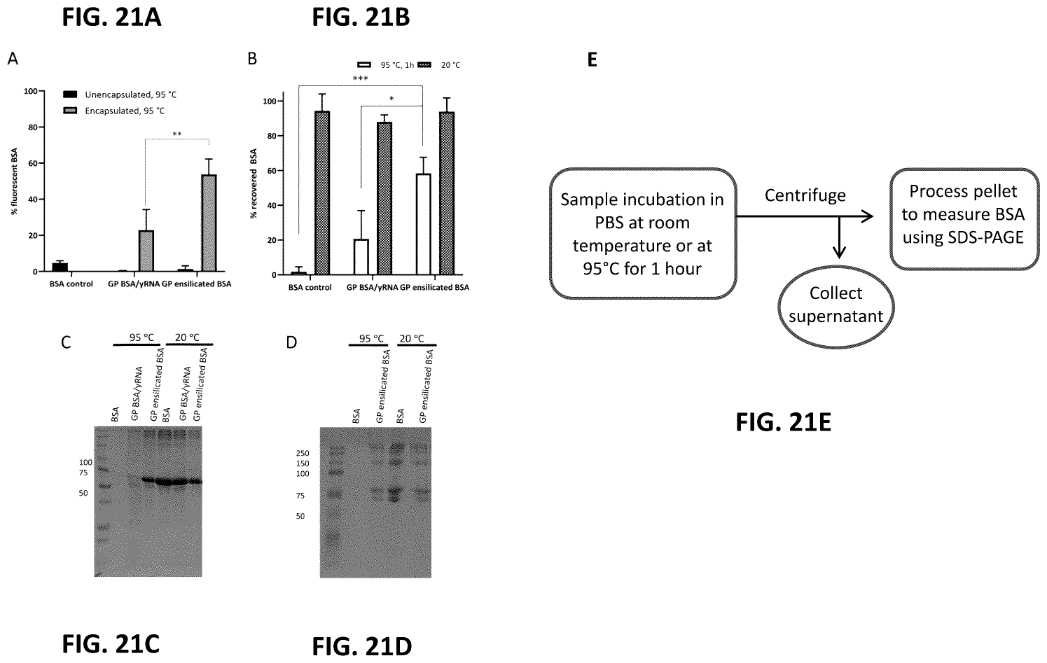

BSA was loaded in GPs as described before. The BSA was then ensilicated by TEOS as described before. GPs containing ensilicated BSA were incubated in PBS at 95° C. for 1 hour (see FIG. 21E). Samples were centrifuged and supernatants discarded. The BSA in the pellet was extracted with 6M Urea and SDS-PAGE loading dye. The extracted BSA was centrifuged. The precipitated protein in the pellet was discarded and BSA remaining in the supernatant was quantified by SDS-PAGE. FIG. 21 shows percent of BSA remaining in GPs after prolonged exposure to high temperature. Free BSA and BSA encapsulated in GPs by trapping with yRNA were used as control. Ensilication provides BSA protection against thermal degradation: FIG. 21A shows recovered BSA quantified by fluorescence in the supernatant of unencapsulated and GP encapsulated BSA samples after heating at 95° C. for 1 h (** p<0.01, n=4). FIG. 21B shows quantification by SDS-PAGE densitometry of recovered BSA from heat treated and control (20° C.) samples (* p<0.1, *** p<0.001, n=3). FIG. 21C shows recovered protein on SDS-PAGE. FIG. 21D shows a native gel showing intact BSA monomer, dimer and trimer recovery from GP ensilicated BSA. Samples were extracted with acidic sodium fluoride solution. Extraction with acidic sodium fluoride does not give quantitative recovery from ensilicated samples. Thus ˜65% of each BSA structure was recovered in sample incubated at 20° C. and ˜38% from sample incubated at 95° C. (percentage values are relative to the free BSA control at 20° C.).

### Example 9: Plug Seal Approach: Silicate Capped GPs

### Production of Silicate Capped YPs

To prepare silicate capped GPs, the method disclosed by T. E. Wagner was adapted (Wagner, T. E.; Vaccine Delivery Systems Using Yeast Cell Wall Particles, US patent application publication No. US20170007688A1).

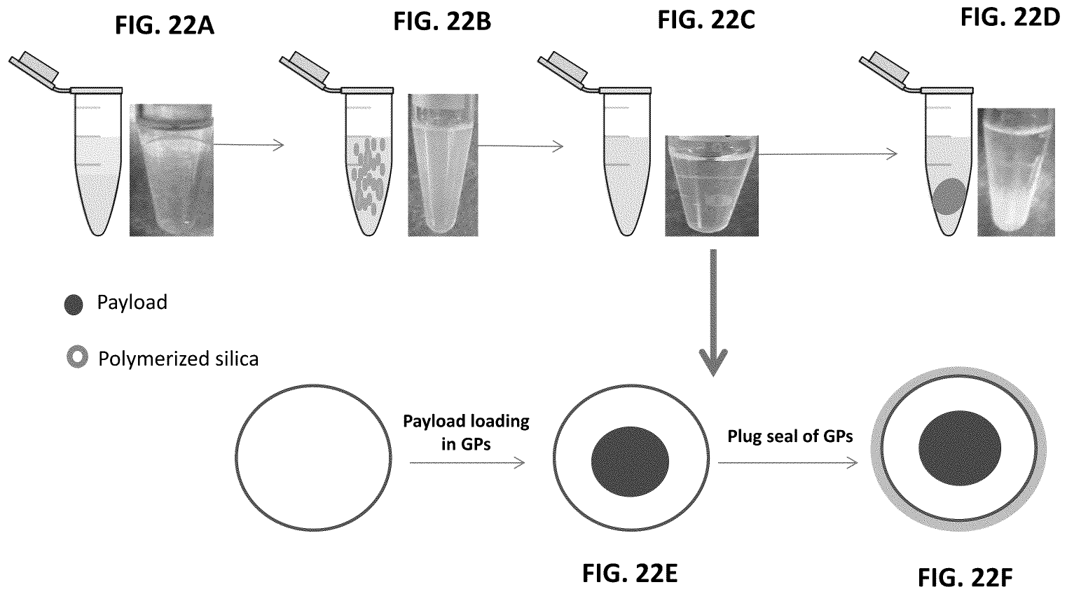

The procedure for production of silicate capped GPs involves several steps which are shown in FIG. 22. Initial TEOS (top phase, d=0.93 g/mL) and aqueous NH₄OH solution (bottom phase) (FIG. 22A) when mixed forms a TEOS-NH₄OH aqueous emulsion composed of partially pre-polymerized TEOS far from gel point (FIG. 22B). Upon further incubation TEOS-aqueous solution forms pre-polymerized TEOS close to gel point (FIG. 22C) which rapidly (<10 minutes) fully polymerizes to form a silica gel (FIG. 22D). When TEOS-NH₄OH aqueous solution close to gel point (FIG. 22C) is incubated with GPs containing payloads (FIG. 22E), the very rapid polymerization of TEOS in basic NH₄OH prevents TEOS from diffusing inside GPs and instead results in polymerization of TEOS on the GP surface resulting in plug sealed or silicate capped GPs (FIG. 22F).

GPs loaded with fluorescent BSA (fBSA) were plug sealed (silicate capped) by the above protocol using 10% NH₄OH as catalyst. GPs containing ensilicated fBSA were also prepared as described before at the optimal TEOS polymerization conditions with 0.01 M HCl (pH 2) for use as control. Percent of BSA trapped in GPs was measured as described before. The plug seal protocol requires a large volume (1 mL per mg GP) and use of ethanol to prevent premature release of BSA from GPs. In contrast, a key difference is that in the method described herein, ensilication is done in half hydrodynamic volume (5 μL per mg GP) trapping the BSA payload inside the GPs.

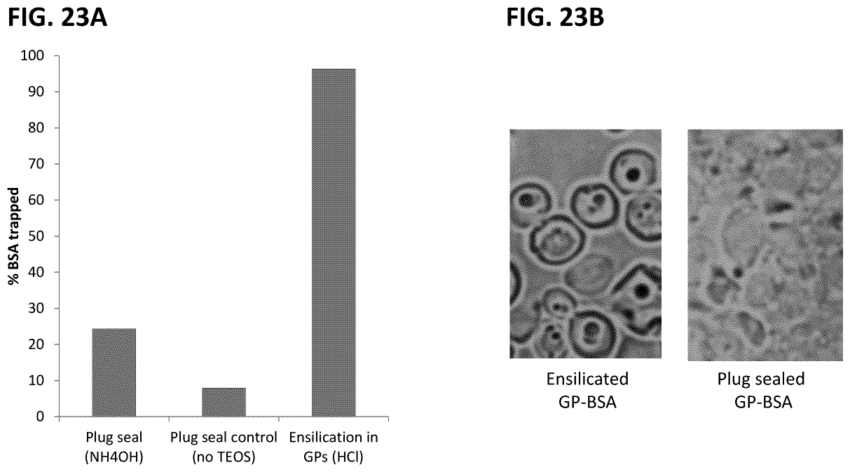

FIG. 23A shows the percent of BSA trapped in GPs in control (no TEOS), ensilication, and plug seal approach. Data indicates that ensilication of BSA within GPs is a significantly better trapping strategy as nearly 100% of the BSA was trapped in GPs using ensilication approach. The plug seal approach was much less efficient in retaining BSA within GPs, as it resulted in trapping of just over 20% of BSA. FIG. 23B shows fluorescent micrographs indicating location of fBSA. The images also show that plug sealed GPs clumped together due to rapid silica gel formation.

### In Vitro Leaking Assay

To detect leaking of payload from plug sealed or silicate capped YPs, an in vitro leaking assay was performed. Dry YPs were loaded with a payload (e.g., peptide or fluorescent albumin) and then were resuspended in ethanol (1 mg YP/mL). TEOS (100 μL) and 10% aqueous ammonia solution (100 μL) was added to the YP suspension. Mixture was incubated 15 min at room temperature. Silicate capped YPs were washed with ethanol and stored in ethanol at 4° C.

An in vitro leaking assay was performed on silicate capped YPs containing fluorescent albumin by the following procedure. Silicate capped YPs containing fluorescent albumin were incubated in PBS. Samples of supernatant were collected at 0, 1 and 2 hour time points. Fluorescent albumin in supernatant was measured. After 1 hour, uncapped YPs leaked 24.73% of fluorescent payload and silicate capped YPs leaked 15.81% of fluorescent payload. After 2 hours, uncapped YPs leaked 16.6% and silicate capped YPs leaked 6.65% of payload. The total loss of payload due to leakage was 41.33% in uncapped YPs but only 22.46% in capped YPs. While uncapped YPs retained 58.67% of the payload protein, capped YPs retained significantly more, 77.54% of the payload.

### Example 10: BSA Ensilication in GPs with NH₄OH as a Catalyst

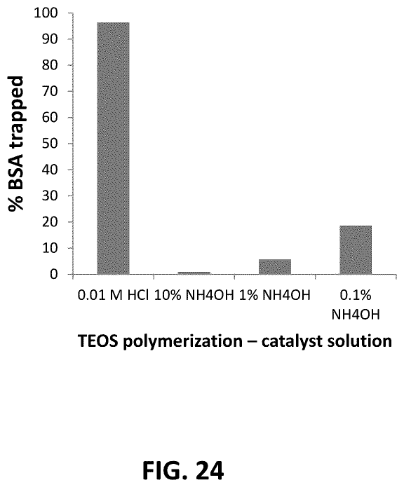

The ability of NH₄OH to catalyze ensilication of BSA within GPs was tested. NH₄OH was added as a polymerization catalyst at a concentration of 0.1%, 1%, and 10% to the mixture of GP-BSA and partially pre-polymerized TEOS. Percent of BSA trapped in GPs was measured as described before and is shown in FIG. 24. Rapid gel formation of TEOS in ammonium hydroxide prevents efficient GP loading of pre-polymerized TEOS.

### Example 11: Use of Ensilicated Payloads in GPs as Vaccines

### Ensilicated OVA and MSA in GPs

GPs were loaded with chicken ovalbumin (OVA) and mouse serum albumin (MSA) payloads. Payloads were either trapped with yRNA or ensilicated with TEOS by the procedure described before.

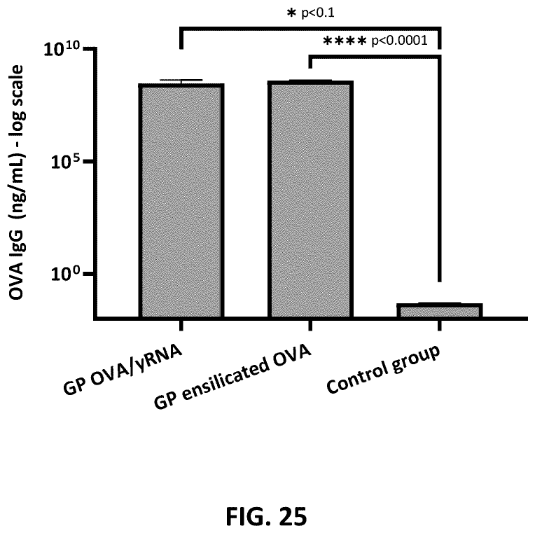

Mice were immunized with intravenous (IV) administration of GPs containing OVA payload trapped within GPs via ensilication or yRNA. Anti-OVA antibodies in mouse serum were measured using ELISA. Mice developed high IgG antibody titers regardless of the method used to trap antigen payload (FIG. 25). The results show that ensilicated GP-OVA-MSA served as effective a vaccine as the control GP-OVA-MSA/yRNA vaccine.

### Ensilicated GP Cryptococcal Vaccine Antigens

Meningitis due to Cryptococcus neoformans is responsible for upwards of 180,000 deaths worldwide annually, mostly in immunocompromised individuals such as AIDS patients. Currently there are no licensed fungal vaccines, and even with anti-fungal drug treatment, cryptococcal meningitis is often fatal. Thus, Cryptococcosis remains a significant cause of morbidity and mortality world-wide.

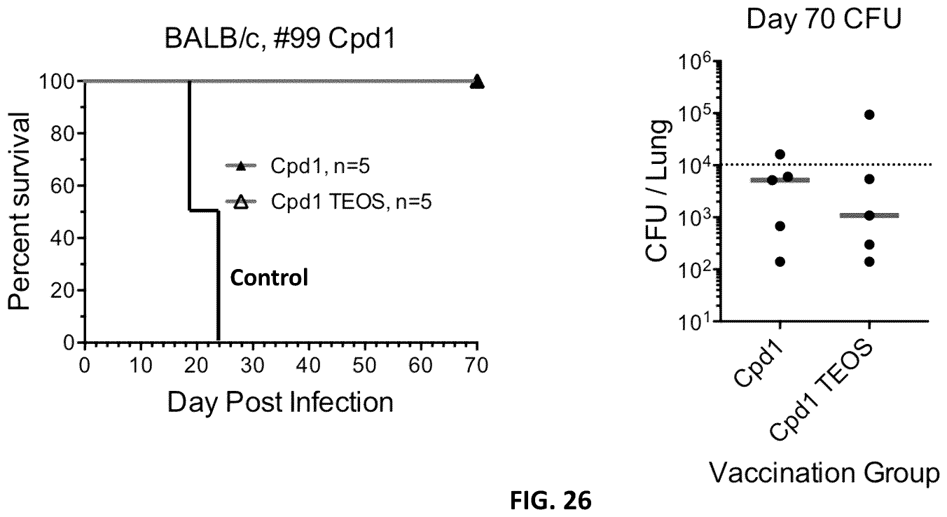

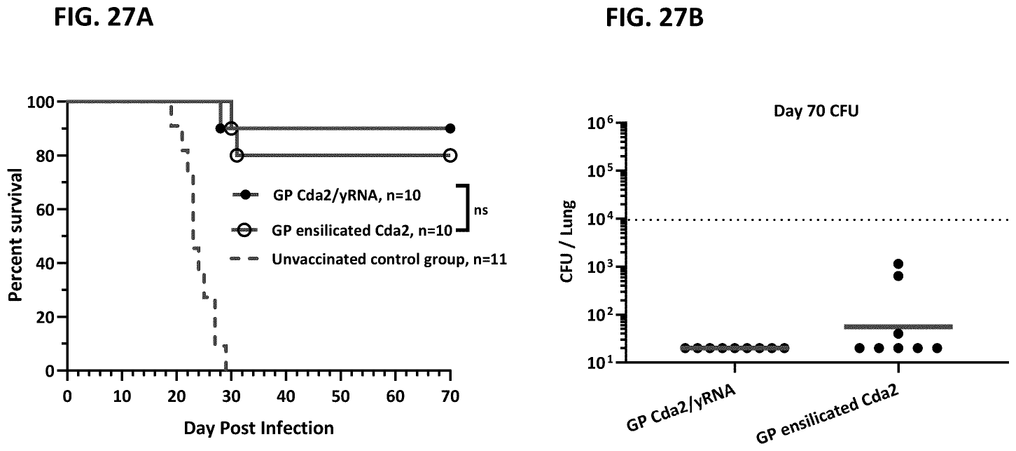

To develop and test the efficacy of cryptococcal vaccines in mice, vaccines were prepared by encapsulating cryptococcal protein antigens carboxy peptidase (Cpd1) or chitin deacetylase (Cda2) in GPs. Antigens were either trapped in GPs using yRNA (GP Cpd1 MSA/yRNA or GP Cda2 MSA/yRNA) or ensilicating antigen in GPs with TEOS (GP Cpd1 TEOS or GP Cda2 TEOS). Mice (BALB/c) were immunized with GPs carrying each single antigen payload. Vaccinated and unvaccinated mice were challenged with a lethal cryptococcal infection and percent survival was observed. FIG. 26A shows that almost all unvaccinated (control) mice died around 25 days post infection while 100% of the mice vaccinated with the Cpd1 antigen (either trapped with yRNA or ensilicated within GPs) survived for 70 days post infection. FIG. 26B shows that in the vaccinated mice that received GPs with ensilicated antigen showed fewer bacterial colony forming units (CFUs) in the lung that mice vaccinated with yRNA trapped antigen. This indicates that ensilicated antigen promoted a stronger protective immunity in vaccinated mice. FIG. 27A shows survival rates of mice vaccinated with the Cda2 antigen in GPs (either trapped with yRNA or ensilicated) and FIG. 27B shows the Cryptococcus colony forming units (CFUs) in mice at day 70. FIG. 27A shows that almost all unvaccinated (control) mice died around 30 days post infection while 80-90% of the mice vaccinated with the Cpd2 antigen (either trapped with yRNA or ensilicated within GPs) survived for 70 days post infection.

### Example 12: Ensilication of Lysozyme (Lys, MW=14 kD)

Ensilication Efficiency with Lysozyme Payload

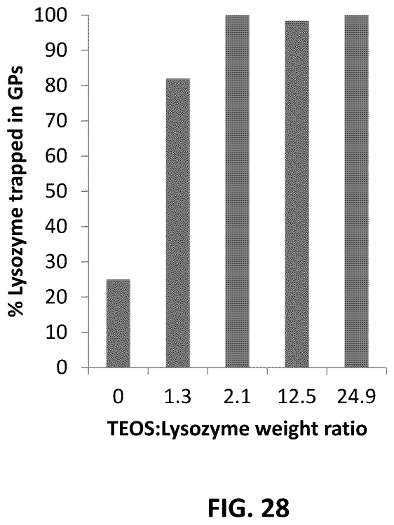

GPs were loaded with lysozyme (Lys) using the same procedure described before for loading of BSA in GPs. TEOS was partially pre-polymerized at pH 2 and the lysozyme payload inside GPs was ensilicated as described before. FIG. 28 shows the efficiency at which lysozyme was ensilicated in GPs at various TEOS:lysozyme w/w ratios.

### Thermal Stability of Ensilicated Lysozyme

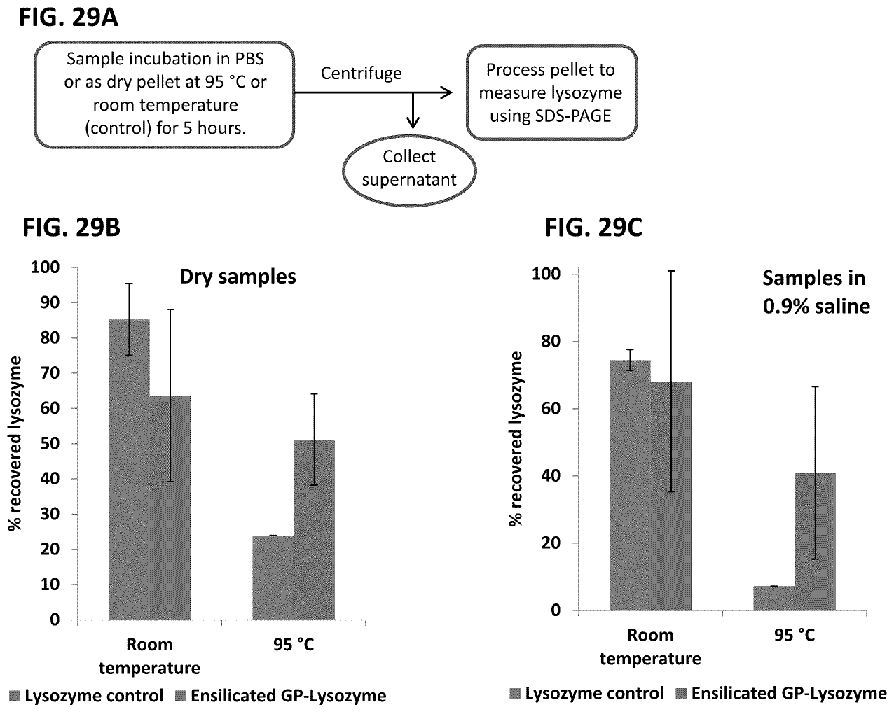

Lysozyme was loaded in GPs by the procedure as described before for BSA. The lysozyme payload was then ensilicated using TEOS (TEOS:Lysozyme ratio 12.5:1) as described before for BSA. GPs containing ensilicated lysozyme were incubated in dry form or in 0.9% saline at 95° C. or room temperature for 5 hours. Samples were centrifuged and supernatants discarded. The BSA in the pellet was extracted with 6M Urea and SDS loading dye. The extracted BSA was centrifuged. The precipitated protein in the pellet was discarded and BSA remaining in the supernatant was quantified by SDS-PAGE. FIG. 29A summarizes the protocol used to evaluate the percent of lysozyme remaining in GPs after prolonged incubation at given temperatures. FIG. 29 shows percent of lysozyme remaining in dry (FIG. 29B) or 0.9% saline+/−heating (FIG. 29C) GP-lysozyme samples after prolonged exposure to room or high temperature. Free BSA and BSA encapsulated in GPs by trapping with yRNA were used as control.

Activity of Ensilicated Lysozyme after Exposure to High Temperature

The activity of the ensilicated lysozyme recovered from GPs after incubation at 95° C. for 5 hours in the experiment above was also tested. Lysozyme activity was quantified by spectrophotometric measurement of p-nitrophenol (λ_{abs}=405 nm) released following reaction of lysozyme with p-nitrophenyl β-D-N,N′,N″-triacetyl-chitotriose. The lysozyme assay used is described by Osawa et al. (Toshiaki Osawa, Yasuo Nakazawa, Lysozyme substrates. Chemical synthesis of p-nitrophenyl O-(2-acetamido-2-deoxy-β-D-glucopyranosyl)-(1→4)-2-acctamido-2-deoxy-β-D-glucopyranoside and its reaction with lysozyme, Biochim. Biophys. Acta, 130 (1966) 56-63.)

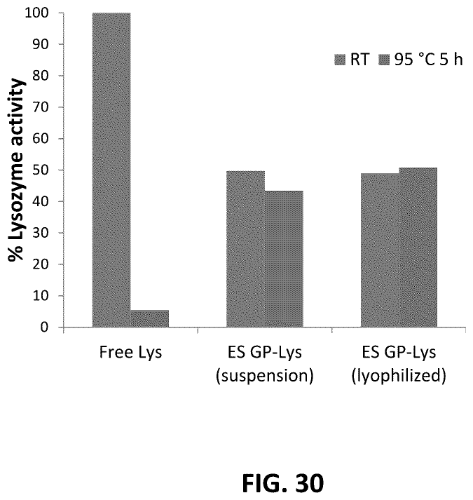

FIG. 30 shows the percent lysozyme activity retained after exposure to high temperature in samples containing free lysozyme or GP with ensilicated lysozyme (either dry/lyophilized sample or sample suspended in 0.9% saline). Results show that ensilication protected lysozyme activity during prolonged high temperature incubation.

### Short Term Storage Stability of GP-Ensilicated Lysozyme

The storage stability of free lysozyme, lysozyme encapsulated within GPs, lysozyme trapped in GPs with yRNA, and lysozyme ensilicated within GPs was tested after incubation at 45° C. for a total of 144 hours. Samples were stored either as dry, lyophilized powders or liquid samples stored in PBS with 2 mM sodium azide (NaN₃). Lysozyme activity was assayed as described before incubation and after 24 hour period.

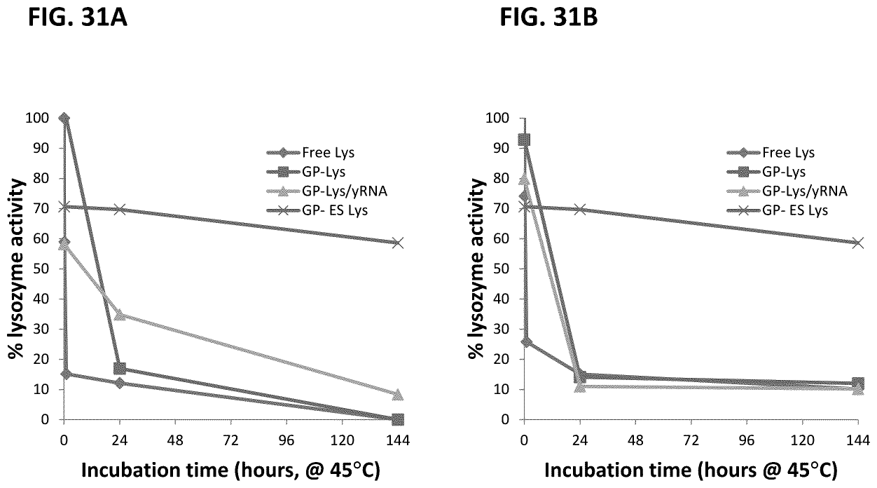

FIG. 31 shows the percent lysozyme activity at various time points during incubation. Free lysozyme (Lys) and GP-Lys samples lost >85% bioactivity within 1-24 h incubation at 45° C. Lysozyme trapped in GPs using yRNA as trapping agent lost >85% bioactivity after 24 hours (sample stored in PBS suspension) or 7 days (dry powder) of incubation at 45° C. GP Ensilicated (ES) lysozyme incubated at 45° C. retained >50% bioactivity after incubation at 45° C. for >7 days. The half-life (T½) for thermal inactivation of free lysozyme was <1 hour and for GP ES LYS was >144 hours.

### Long Term Storage Stability of GP-Ensilicated Lysozyme

The storage stability of free lysozyme and lysozyme ensilicated within GPs was tested after incubation at 45° C. for a total of 90 days. Samples were stored either as dry, lyophilized powders or liquid samples stored in PBS with 2 mM sodium azide (NaN₃). Lysozyme activity was assayed as described before incubation and after 15 days.

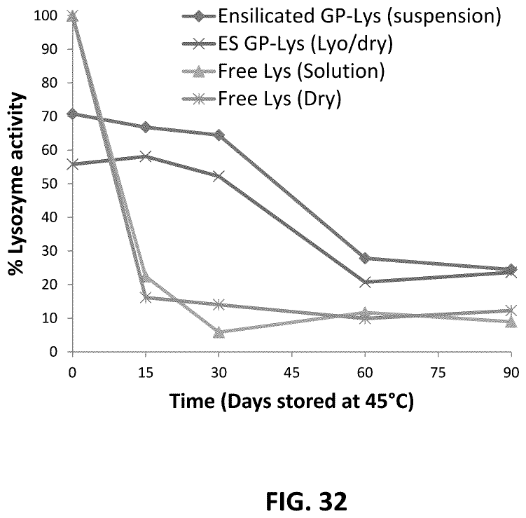

FIG. 32 shows the percent lysozyme activity at various time points during incubation. GP ensilicated lysozyme incubated at 45° C. retained >50% bioactivity for >30 days.

### Example 13: Ensilication of Other Payloads

### Ensilication of IgG Antibody (MW=150 kD)

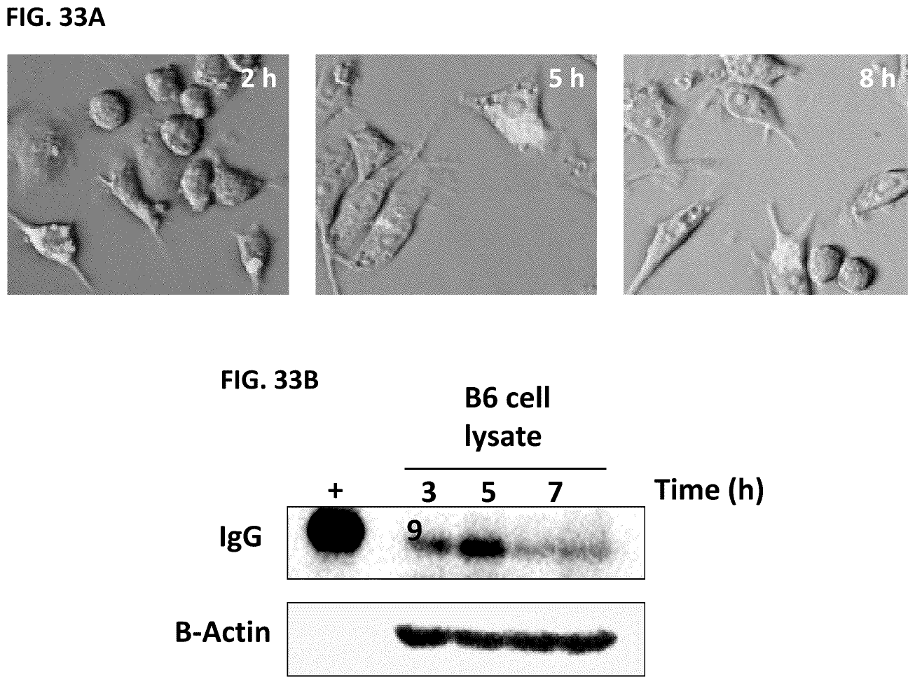

Antibodies (IgG 488) were loaded and ensilicated within GPs by the same process described before for BSA. GPs loaded with ensilicated IgG (IgG 488-(SiO₂)n) were allowed to be phagocytosed by cells of the B6 cell line. FIG. 33A shows that IgG was released inside macrophage cells and diffused throughout the cytoplasm. FIG. 33B shows that intact IgG was detected in B6 cells incubated with GP-IgG-(SiO₂)n.

### Ensilication of Peptides

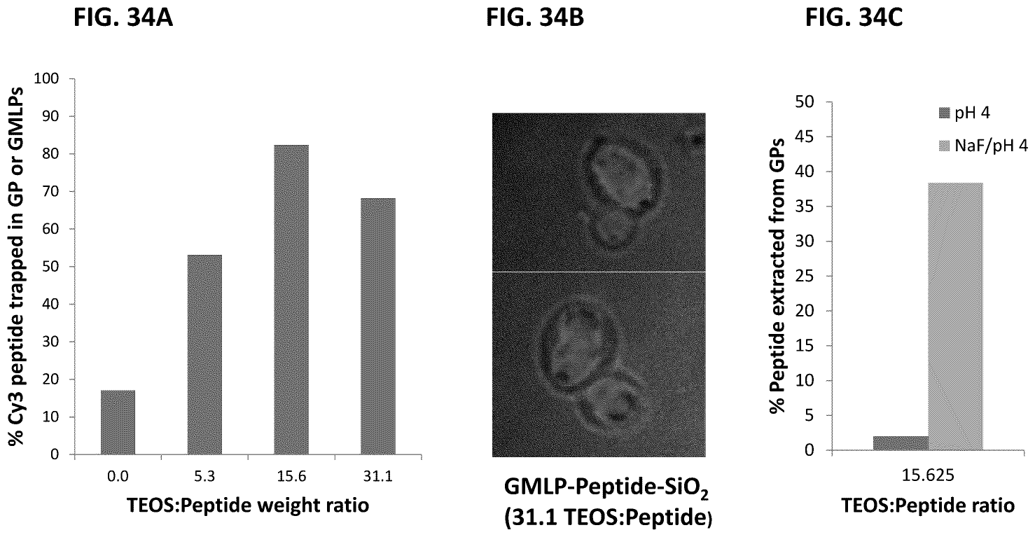

Hairpin peptide (MW 4570.96) and Cda2 Peptide 1 were loaded and ensilicated within GPs or glucan mannan lipid particles (GMLPs) by the same process described above for BSA. Control (0) and 15.6 TEOS samples were prepared in GPs, 5.3 and 31.1 samples were prepared in GMLPs. Percent of peptide ensilicated within GPs was assessed at various TEOS:peptide weight ratios. Over 80% of hairpin peptide was ensilicated within GPs when the TEOS:peptide ratio was 15.6 (FIG. 34A). Fluorescent micrograph showed that the peptide was trapped within GMLPs (FIG. 34B). Hairpin peptide ensilicated within GPs using a TEOS:peptide ratio of 15.6 was extracted from GPs at pH 4 using hydrochloric acid (HCl) or sodium fluoride (NaF). Extraction was most efficient when NaF was used (FIG. 34C).

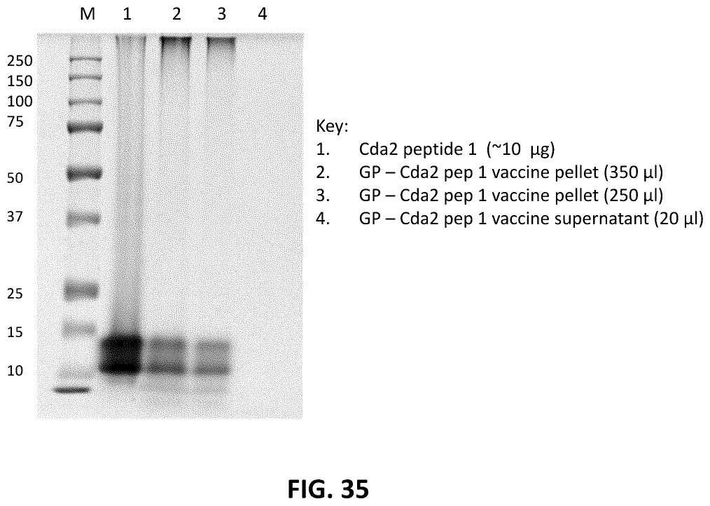

Cda2 peptide 1 was also ensilicated efficiently in GPs as it was detected in the GP pellet but not in the supernatant (FIG. 35).

### Ensilication of Nucleic Acids

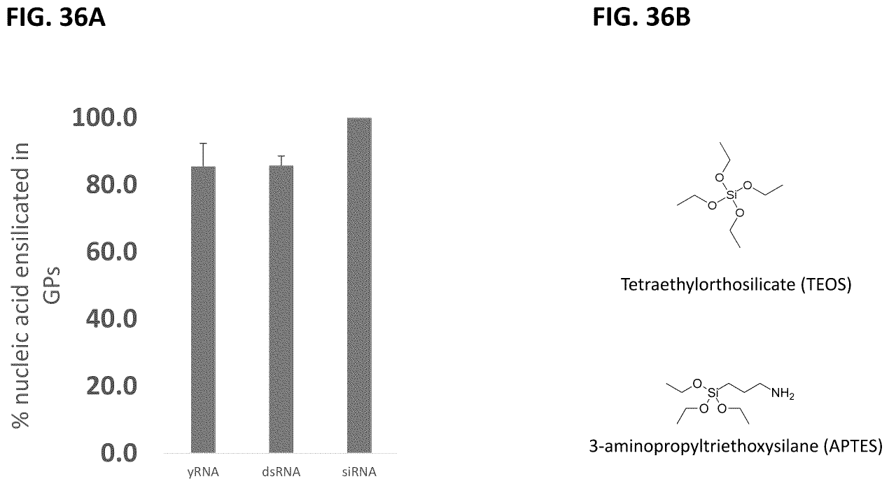

Payloads comprising of yRNA, dsRNA, or siRNA were loaded and ensilicated within GPs by the same process described before for BSA but using 90% TEOS and 10% APTES (see molecular structures of TEOS and APTES in FIG. 36B). Ensilication was carried out in the presence of pH 2 or pH 4 HCl. FIG. 36A shows that all payloads were efficiently ensilicated within GPs. Ensilication of RNA with TEOS yields 50-65% encapsulation whereas encapsulation of RNA with a mixture of 90% TEOS and 10% APTES improves encapsulation efficiency to >85%.

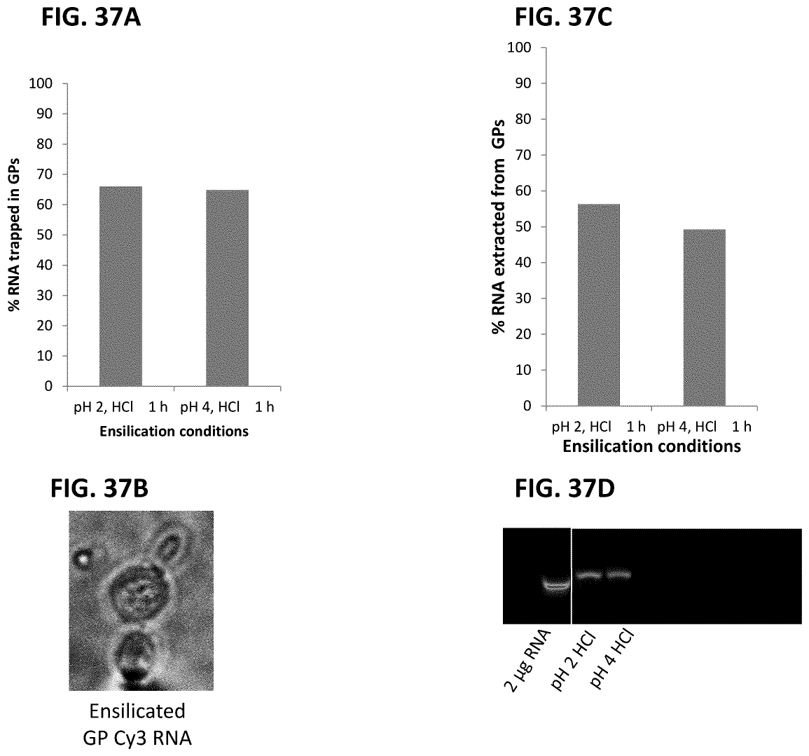

A 300-mer Cy3 RNA, an FITC labeled single stranded DNA (ssDNA), and Cy3-siRNA were loaded and ensilicated within GPs using 100% TEOS by the same process described before for BSA. Ensilication was carried out in the presence of pH 2 or pH 4 HCl. After ensilication, intact RNA was extracted from GPs using 200 mM NaF at pH 4. Ensilication was equally efficient in HCl at pH 2 and 4 (FIG. 37A). Fluorescent micrographs showed that RNA was located inside GPs (FIG. 37B). RNA ensilicated in HCl at pH 2 or pH 4 was efficiently extracted with NaF (FIGS. 37C and 37D).

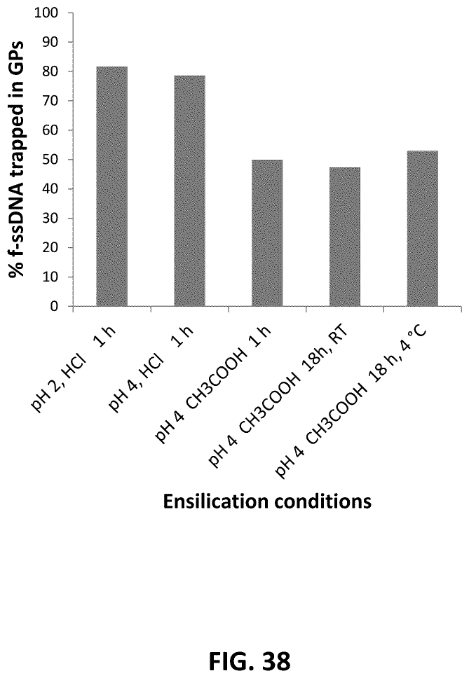

FITC labeled ssDNA was also efficiently ensilicated within GPs when TEOS was partially pre-polymerized at pH 4 in the presence of HCl or acetic acid (CH₃COOH). TEOS partially pre-polymerized in acetic acid was incubated with payload containing GPs at room temperature (RT) or 4° C. for 1 hour or 18 hours (FIG. 38).

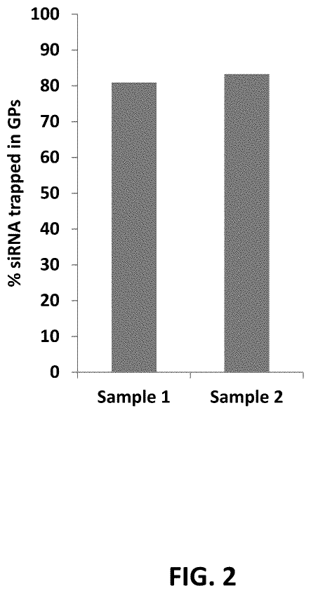

Similarly, Cy3-siRNA was also efficiently ensilicated within GPs using TEOS partially pre-polymerized at pH 2 using HCl (FIG. 2).

### Example 14: Functionalization of YP Surface for Enhanced Uptake

### Preparation of PDA-fGPs

Chemical modification of YP surface with polydopamine (PDA) coat to facilitate intestinal mucin penetration and provide reactive groups for intestinal cell surface receptor ligand attachment for targeted epithelial cell delivery is described in detail by Soto et al. (Polydopamine Coating of Glucan Particles Increases Uptake into Peyer's Patches, ACS Appl. Bio Mater. 2019, 2, 9, 3748-3754) the disclosure of which is incorporated herein in its entirety.

### Example 15: Surface Derivatization of GPs with Epithelial Cell Receptor Ligands to Enhance Uptake

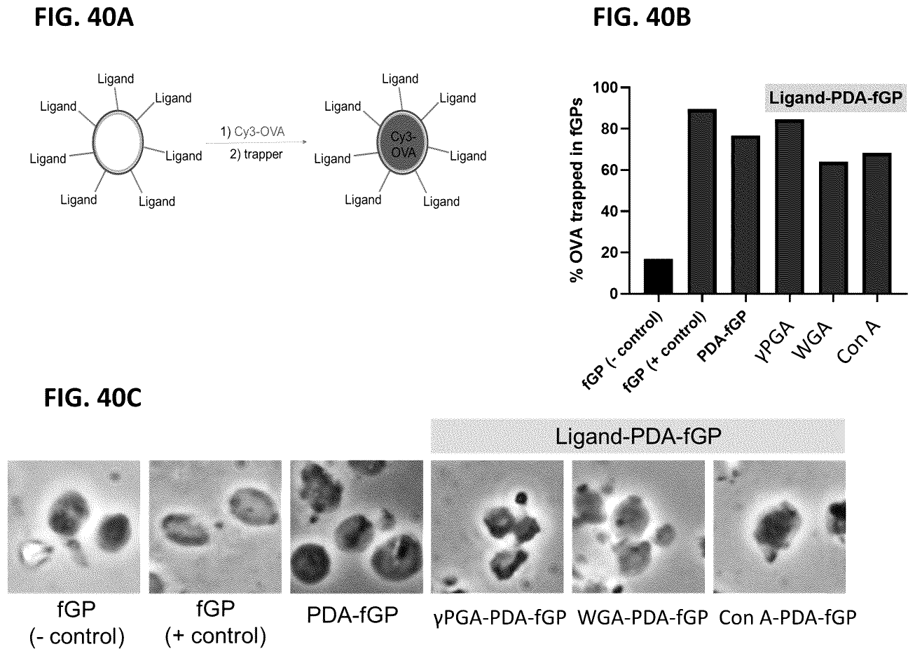

A series of potential ligands identified from the literature were covalently linked to the PDA coated fluorescently labeled YPs (PDA-fYP). Payload loading ability of surface derivatized YPs was tested. As shown in FIG. 40, surface derivatization did not affect the particle loading capacity using fluorescently labeled ovalbumin (Cy3-OVA) as a model payload. Surface derivatized fYPs trapped almost as much ovalbumin as fYPs or PDA-coated fYPs (FIG. 40B). Fluorescent photomicrographs (FIG. 40C) corroborated that fluorescently labeled ovalbumin was located inside the fYPs.

Surface derivatized YPs were screened for binding to an intestinal epithelial cell line in vitro.

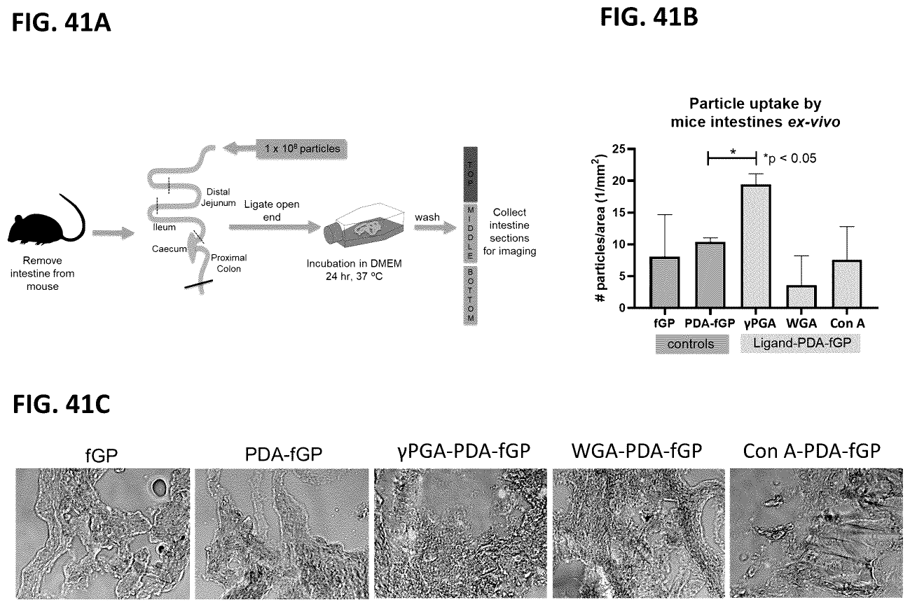

Six potential receptor ligands were selected for ex vivo evaluation in an intestinal loop model (FIG. 41A). The selected ligands were:
γ-PGA: gamma-polyglutamic acid
WGA: wheat germ agglutinin
ConA: concanavalin-A,;

As schematically depicted in FIG. 41A, Intestines from mice were ligated on the large intestinal end and samples of the control and test Ligand-PDA-YCWP-fluorescent formulations were added into the lumen of small intestinal end of the loop. The small intestinal end was ligated to form a sealed tube. After incubation for 18 hours the tissue was fixed, sectioned and imaged.

FIG. 41B shows that GPs carrying γ-PGA ligand on their surface showed 2-fold increased uptake compared to control GPs, i.e., fGPs and PDA-fGPs without any surface ligand.

FIG. 41C shows fluorescent photomicrograph showing enhanced uptake of GPs carrying γ-PGA ligand on their surface by the mouse intestinal cells. This indicates that ligands on GP surface provide increased mucosal barrier penetration.

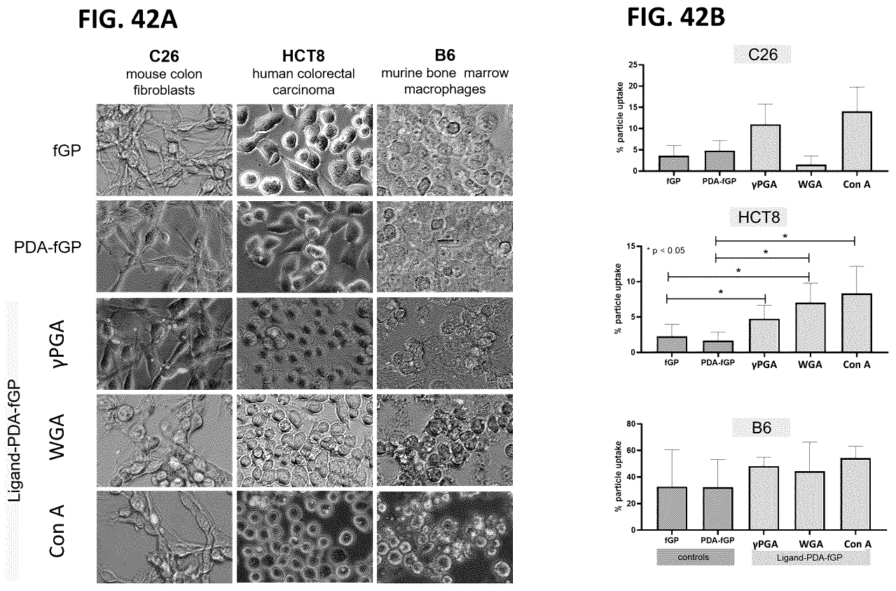

FIG. 42 shows uptake of fGP, PDA-fGPs and ligand-PDA-fGPs by three different cell lines in vitro. For all cell lines tested, the percent uptake of ligand-PDA-fGPs was 2 to 3 fold higher than control GPs without surface ligands.

### Example 16: In Vivo Uptake and Biodistribution of Surface Derivatized GPs

Further in vivo studies were carried out using ligands A-gamma-polyglutamic acid, γ-PGA; E-Arginylglycylaspartic acid (RGD) peptide and D-. C-terminal Src kinase (CSK) peptide (CSK)

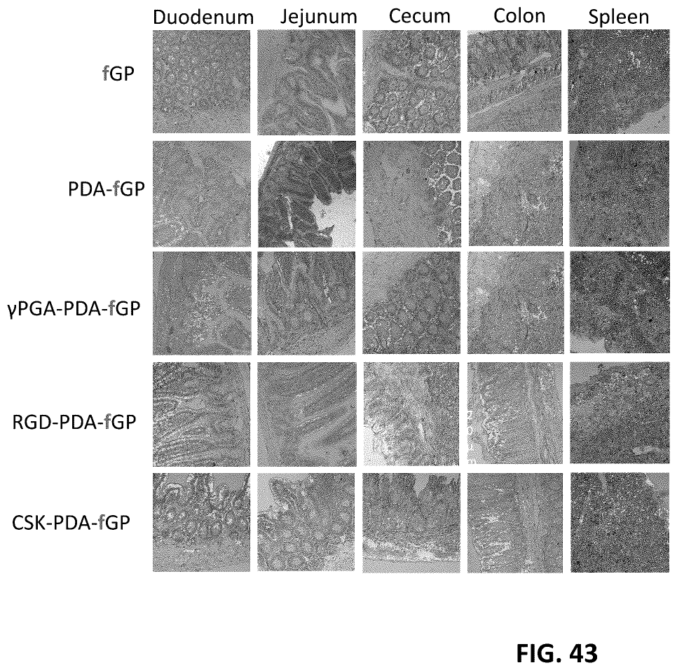

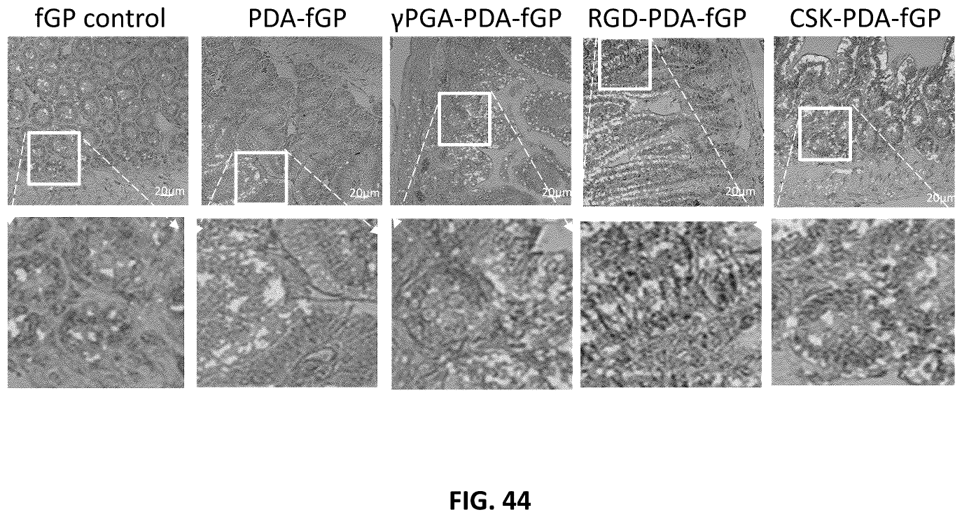

Control PDA-fGP and ligand-PDA-fGP formulations with 1×10⁸ particles (200 μg) of surface modified GPs were gavaged into mice. Mice were sacrificed after 24 hours, and stomach, intestines, lymph nodes, spleens and livers were collected, fixed sectioned and imaged. The imaging results of the tissues evaluated qualitatively show that PDA-fGPs surface derivatized with any of the three ligands were better taken up into intestinal epithelial cells, underlying GALT tissues, and spleen (see FIGS. 43 and 44). The CSK ligand appeared to work better than the RGD and gamma-polyglutamic acid ligands.

### Example 17: Enteric Coating of YPs for Payload Protection from Release and/or Degradation in the Digestive Tract

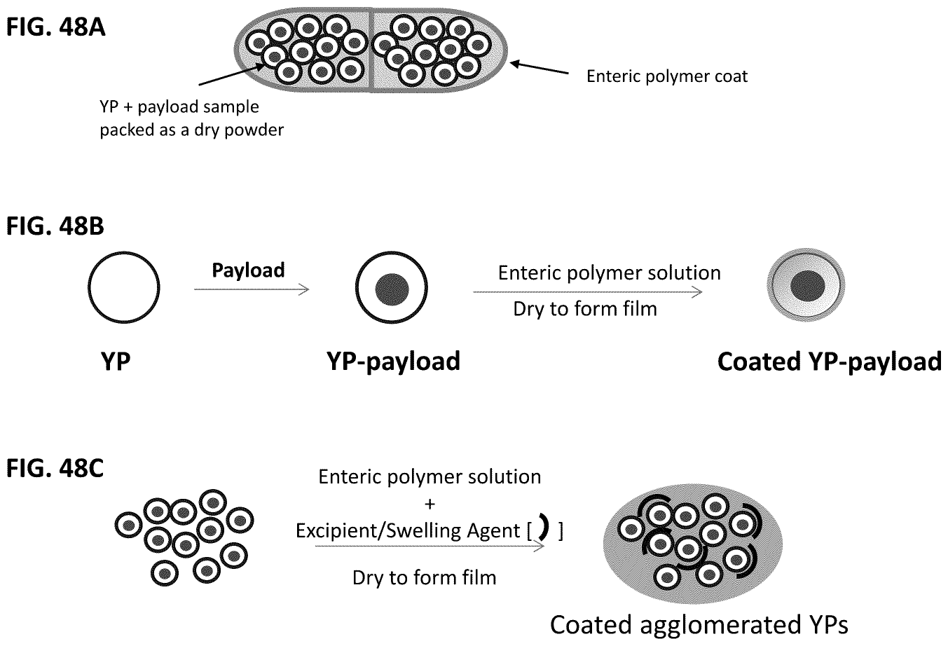

YPs can be packed in commercially available enteric coated capsules as shown in FIG. 48A.

YPs can be individually coated in a hydrodynamic volume of enteric polymer as shown in FIG. 48B. Enteric coating polymer solution is added to YPs and allowed to dry to form a film of enteric polymer around each individual YP. One or multiple coats of the polymer can be applied to the individual YPs to maximize payload protection. Example of single particle coating is also described in Example 7 and 8 and depicted in FIG. 20. Eudragit coated GPs with ensilicated BSA better protect the payload when exposed to SGF.

Alternatively, agglomerated YPs can be coated with an enteric polymer as shown in FIG. 48C. The enteric polymer solution is added to the YPs along with an excipient or swelling agent and subsequently dried form a film of enteric polymer around agglomerated YPs. The excipient acts as a swelling agent to disaggregate YPs after dissolution of the enteric coat. Swelling agents such as hydroxypropyl methylcellulose (HPMC) can be used.

### Example 18: Ensilication within GPs of Proteins with Different Molecular Weights and Isoelectric Points

The experimental procedure described in Example 3 was further optimized for protein ensilication at a TEOS:protein weight ratio of 12.4:1. Ensilication efficiency was tested using payloads comprising lysozyme (Lys), ovalbumin (OVA), transferrin (TFN), and glucose oxidase (GOx).

20 μL of a 25 mg of protein in aqueous solution was added to a dry sample of 5 mg GPs. The samples were mixed, incubated at room temperature for 30 minutes, frozen and lyophilized to remove solvent. The loading cycle was repeated by adding 10 μL of water to the dry GP protein sample, the sample was mixed and lyophilized to remove solvent.

TEOS was mixed with 0.01 M HCl solution (pH 2). The mixture was mixed for 1 hour to achieve TEOS pre-polymerization and formation of a single aqueous phase in which the TEOS was close to gel point. Solutions with different ratios of TEOS and 0.01 M HCl were prepared to target TEOS:protein weight ratios of 12.4. The partially pre-polymerized TEOS mixture was added to the dry GP-protein pellet (20 μL/5 mg GP). The sample was mixed and incubated at room temperature for 1 hour.

The ensilicated fGP-rBSA samples were washed three times with 0.9% saline. The percent protein encapsulation was quantified by SDS-PAGE. GPs containing non-ensilicated lysozyme, ovalbumin, transferrin, and glucose oxidase were used as control. GOx was loaded and encapsulated as dimer (160 k). It is extracted and quantified as monomer by SDS-PAGE as a protein denaturing solution is necessary for quantitative extraction.

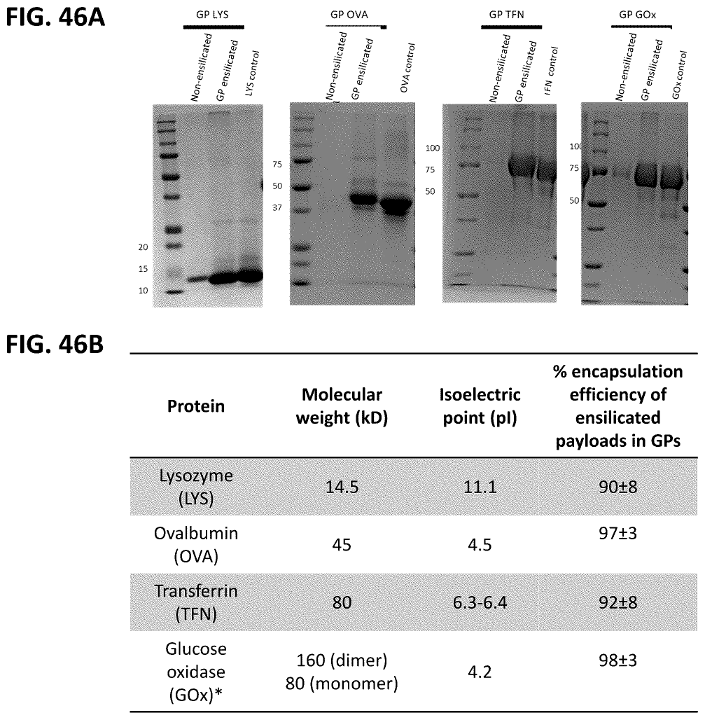

FIG. 46 shows that ensilication of lysozyme, ovalbumin, transferrin, and glucose oxidase allowed their encapsulation into GPs at over >90% encapsulation efficiency. This shows that proteins with varying molecular weight and isoelectric points can be encapsulated within GPs with ensilication.

### Example 19: Transfection Using RNA Ensilicated within GPs

5 μL of a 1 mg mCherry mRNA/mL aqueous solution was added to a dry sample of 1 mg GPs. The samples were mixed, incubated at 4° C. for 30 minutes, frozen and lyophilized to remove solvent. The loading cycle was repeated by adding 10 μL of water to the dry GP protein sample, the sample was mixed and lyophilized to remove solvent.

A mixture of 10% volume APTES and 90% volume TEOS was mixed with 0.01 M HCl solution (pH 2). The mixture was mixed for 15 minutes to achieve APTES and TEOS pre-polymerization and formation of a single aqueous phase in which the APTES/TEOS mixture was close to gel point. The pre-polymerized solution was prepared to target (TEOS+APTES):mRNA weight ratio of 12.4. The partially pre-polymerized APTES/TEOS mixture was added to the dry GP-mRNA pellet (4 μL/1 mg GP). The sample was mixed and incubated at room temperature for 1 hour.

GP yRNA/PEI/mCherry mRNA control sample was prepared as previously described in the literature for GP RNA core and layer-by-layer formulations (Soto et al (2008), Bioonjug. Chem. 19 (4), 840-848; Aouadi et al. (200) Nature 458 (7242): 1180-4).

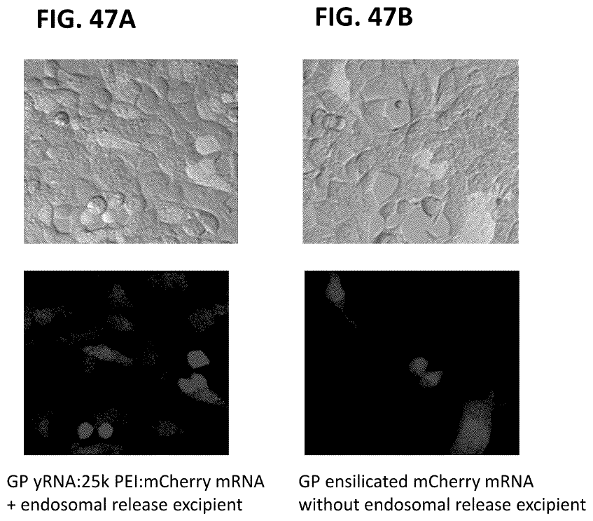

Samples of GP yRNA/PEI/mCherry mRNA and GP ensilicated mCherry mRNA in 0.9% saline were added to 3T3-D1 cells in DMEM at a GP:cell ratio of 5:1. The samples were incubated 24 h at 37° C., 5% CO₂. Cells were imaged with a confocal microscope to evaluate mCherry protein expression by fluorescence of mCherry protein. FIG. 47 shows the fluorescence photomicrographs of mCherry protein expression in 3T3-D1 cells with (A) or without (B) brightfield micrograph overlay.

### LITERATURE CITED

Toshiaki Osawa, Yasuo Nakazawa, Lysozyme substrates. Chemical synthesis of p-nitrophenyl O-(2-acetamido-2-deoxy-β-D-glucopyranosyl)-(1→4)-2-acetamido-2-deoxy-β-D-glucopyranoside and its reaction with lysozyme, Biochimica et Biophysica Acta (BBA)—General Subjects, Volume 130, Issue 1, 1966, Pages 56-63, ISSN 0304-4165, https://doi.org/10.1016/0304-4165 (66) 90008-0.
Buckley, A. M., & Greenblatt, M. (1994). The sol-gel preparation of silica gels. Journal of Chemical Education, 71 (7), 599-602.
Chen, Y C., Smith, T., Hicks, R. et al. Thermal stability, storage and release of proteins with tailored fit in silica. Sci Rep 7, 46568 (2017). https://doi.org/10.1038/srep46568.
Wahid, A. A., Doekhie, A., Sartbaeva, A. et al. Ensilication Improves the Thermal Stability of the Tuberculosis Antigen Ag85b and an Sbi-Ag85b Vaccine Conjugate. Sci Rep 9, 11409 (2019). https://doi.org/10.1038/s41598-019-47657-9.
Aouadi M, Tesz G J, Nicoloro S M, Wang M, Chouinard M, Soto E, Ostroff G R, Czech M P. Orally delivered siRNA targeting macrophage Map4k4 suppresses systemic inflammation. Nature. 2009 Apr. 30; 458 (7242): 1180-4. doi: 10.1038/nature07774. PMID: 19407801; PMCID: PMC2879154.
Soto, E; Ostroff, G; Oral Macrophage Mediated siRNA Delivery System. Nanotech 2008 Volume 2, Chapter 4—Drug and Gene Delivery Systems, pages 332-335.
Ernesto R. Soto and Gary R. Ostroff. Characterization of Multilayered Nanoparticles Encapsulated in Yeast Cell Wall Particles for DNA Delivery Bioconjugate Chemistry 2008 19 (4), 840-848 DOI: 10.1021/bc700329p
Soto, E. R; Ostroff, G. R; Yeast Cell Wall Particles As A Versatile Macromolecular Delivery System. PMSE Preprints (2008), 98 591-593.
SOTO, E; OSTROFF, G. R; Oral Macrophage Mediated Gene Delivery System. Nanotech 2007 Volume 2, Chapter 5-Drug Delivery, pages 378-381.
Soto, E; Ostroff, G; Glucan Particles as Carriers of Nanoparticles for Macrophage-Targeted Delivery, Nanomaterials for Biomedicine, Chapter 3, 2012, pp 57-79, ACS Symposium Series, Volume 1119.
Soto E R, Caras A C, Kut L C, Castle M K, Ostroff G R. Glucan particles for macrophage targeted delivery of nanoparticles. J Drug Deliv. 2012; 2012:143524. doi: 10.1155/2012/143524. Epub 2011 Oct. 13. PMID: 22013535; PMCID: PMC3195348.
Soto, E; Rus, F; Ostroff, G. Yeast Cell Wall Particle Encapsulation of Pro-Terpene Payloads, 2019, TechConnect Briefs 2019, 435-438
Calo, J. R.; Crandall, P. G.; O'Bryan, C. A.; Ricke S. C. Essential Oils as Antimicrobials in Food Systems-A Review. Food Control. 4602015, 54, 111-119.
Bakry, A. M.; Abbas, S.; Ali, B.; Majeed, H.; Abouelwafa, M. Y.; Mousa, A.; Liang, L. Microencapsulation of Oils: A Comprehen-462sive Review of Benefits, Techniques, and Applications. Compr. Rev. Food Sci. Food Saf. 2016, 15, 143-182. 463.
Bhalerao, Y. P.; Wagh S. J. A Review on Thymol Encapsulation and its Controlled Release through Biodegradable Polymer Shells. 465Int. J. Pharm. Sci. 2018, 2, 4522-4532. doi: 10.13040/IJPSR.0975-8232.9 (11).4522-32 466.
Gómez, B.; Barba, F. J.; Domínguez, R.; Putnik, P.; Bursać Kovačević, D.; Pateiro, M.; Toldrá, F.; Lorenzo, J. M. Microencapsula-467tion of Antioxidant Compounds through Innovative Technologies and Its Specific Application in Meat Processing. Trends Food 468Sci. Technol. 2018, 82, 135-147.
Saifullah, M.; Shishir, M.; Ferdowsi, R.; Rahman, M.; Van Vuong, Q. Micro and Nano Encapsulation, Retention and Controlled Release of Flavor and Aroma Compounds: A Critical Review. Trends Food Sci. Technol. 2019, 86, 230-251. 471.
Soto, E., Kim, H., De Jesus, Y. M., Ostroff, G. Polydopamine Coating of Glucan Particles Increases Uptake into Peyer's Patches, ACS Appl. Bio Mater. 2019, 2, 9, 3748-3754. DOI: 10.1021/acsabm.9b00379
Wagner, T. E, Vaccine Delivery Systems Using Yeast Cell Wall Particles, 2017, U.S. Pat. No. 10,166,195, and United States Patent Application publication 2017/0007688 A1
Poinard, B.; Kamaluddin, S.; Tan, A.; Neoh, K.; Kah, J. Polydopamine Coating Enhances Mucopenetration and Cell Uptake of Nanoparticles. ACS Appl. Mater. Interfaces 2019, 11, 4777-4789.
Soto, E. R.; Rus, F.; Li, H.; Garceau, C.; Chicca, J.; Elfawal, M.; Gazzola, D.; Nielsen, M. K.; Urban, J. F., Jr.; Aroian, R. V.; Ostroff, G. R. Yeast Particle Encapsulation of Scaffolded Terpene Compounds for Controlled Terpene Release. Foods 2021, 10, 1207. https://doi.org/10.3390/foods10061207

1. A gastric resistant, enteric coated nano-silica yeast particle (YP) delivery system comprising:
(i) a YP with a hollow inner cavity and at least one first payload,
wherein the at least one first payload is substantially encapsulated by a nano-silica cage, optionally wherein the at least one first payload is completely encapsulated by a nano-silica cage; and
wherein the nano-silica cage and the at least one first payload are both confined within the hollow inner cavity of the YP;
(ii) the YP surface is modified with a mucus penetrating layer, optionally a polydopamine (PDA) layer;
(iii) the YP surface is further modified to display a receptor ligand; and
(iv) The YP is coated with an enteric coat.

2. (canceled)

3. The gastric resistant, enteric coated nano-silica yeast particle (YP) delivery system of claim 1, wherein the YP is selected from the group consisting of a yeast cell wall particle (YCWP), a glucan particle (GP), a yeast glucan particle (YGP), a yeast glucan-mannan particle (YGMP), a glucan lipid particle (GLP), a whole glucan particle (WGP), a glucan mannan lipid particle (GMLP), a glucan chitin particle (GCP) and a mixture thereof.

4. The gastric resistant, enteric coated nano-silica yeast particle (YP) delivery system of claim 1, wherein the at least one first payload is selected from the group consisting of a protein, a peptide, a peptide antigen, an enzyme, an antibody, a nanobody, an antigen binding fragment of an antibody, a single stranded nucleic acid, a double stranded nucleic acid, and any mixtures thereof.

5. The gastric resistant, enteric coated nano-silica yeast particle (YP) delivery system of claim 1, wherein the nano-silica cage comprises a chemical selected from the group consisting of tetraethylorthosilicate (TEOS), tetraethylorthogermanate (TEOG), tetramethylorthosilicate (TMOS), aminopropyl triethoxysilicate (APTES), 3-(triethoxysylil)-propyl-isocyanate (TEPI), bis[3-triethoxysilyl) propyl]disulfide (BTEPDS), bis[3-(triethoxysilyl)propyl]tetrasulfide (BTEPTS) and any combinations thereof.

6-7. (canceled)

8. The gastric resistant, enteric coated nano-silica yeast particle (YP) delivery system of claim 1, further comprising a coating polymer,
wherein the coating polymer is located on the outside of the nano-silica cage, or on the outside of the YP,
wherein the coating polymer is nontoxic and has no pharmacologic activity.

9. The gastric resistant, enteric coated nano-silica yeast particle (YP) delivery system of claim 8, wherein the coating polymer resists breakdown in the presence of gastric fluids in the oral cavity, esophagus, stomach, or small intestine.

10. (canceled)

11. The gastric resistant, enteric coated nano-silica yeast particle (YP) delivery system of claim 8, where in the coating polymer is selected from the group consisting of methacrylic acid methylmethacrylate copolymer, methacrylic acid ethyl acrylate copolymer, cellulose acetate phthalate (CAP), cellulose acetate trimellate (CAT), hydroxy propyl methyl cellulose phthalate (HPMCP), hydroxyl propyl methyl cellulose acetate succinate (HPMCAS), polyvinyl acetate (PVAP), methacrylic acid polymer, and any combinations thereof.

12. The gastric resistant, enteric coated nano-silica yeast particle (YP) delivery system of claim 1, wherein the payload is stable after a short-term or a long-term exposure to high temperature,
optionally wherein the payload is stable after exposure to a temperature of 25° C., 45° C., or 95° C.,
optionally wherein the payload is stable after exposure to the said high temperature for about 30 minutes, about 2 hours, about 5 hours, 15 days, 30 days, 45 days, 60 days, 75 days or 90 days.

13-14. (canceled)

15. The gastric resistant, enteric coated nano-silica yeast particle (YP) delivery system of any one of claim 1 further comprising a second payload, wherein the second payload is not confined in the nano-silica cage,
optionally wherein the second payload is selected from the group consisting of a protein, a peptide, a peptide antigen, an enzyme, an antibody, an antigen binding fragment of an antibody, a single stranded nucleic acid, a double stranded nucleic acid, and a mixture thereof.

16. (canceled)

17. The gastric resistant, enteric coated nano-silica yeast particle (YP) delivery system of claim 1, wherein the receptor ligand is an intestinal epithelial cell surface receptor ligand,
optionally wherein the receptor ligand is an intestinal epithelial cell surface receptor ligand selected from a group consisting of gamma-polyglutamic acid (γ-PGA), wheat germ agglutinin (WGA), concanavalin-A, (ConA), C-terminal Src kinase (CSK) peptide, arginylglycylaspartic acid (RGD) peptide, Fc fragment and a combination thereof.

18. (canceled)

19. The gastric resistant, enteric coated nano-silica yeast particle (YP) delivery system of claim 1, wherein the enteric coat is a polymer,
optionally wherein the enteric coat is a polymer selected from a group consisting of cellulose acetate phthalate (CAP), cellulose acetate trimellate (CAT), hydroxy propyl methyl cellulose phthalate (HPMCP), hydroxyl propyl methyl cellulose acetate succinate (HPMCAS), polyvinyl acetate (PVAP), and methacrylic acid polymers.

20. (canceled)

21. The gastric resistant, enteric coated nano-silica yeast particle (YP) delivery system of claim 1 and a pharmaceutically acceptable carrier or excipient.

22. A kit comprising gastric resistant, enteric coated nano-silica yeast particle (YP) delivery system of claim 1 and optional instructions for use.

23. A method of preparing a gastric resistant, enteric coated nano-silica yeast particle (YP) delivery system comprising the steps of:
(a) loading a YP comprising a hollow inner cavity with at least one first payload; and
(b) resuspending the YP in partially pre-polymerized tetrahydroorthosilicate (TEOS) in half hydrodynamic volume,
wherein the partially pre-polymerized TEOS is partially pre-polymerized at a pH of about 2 to about 4, wherein the TEOS polymerizes to form a nano-silica cage within the hollow inner cavity, and
wherein the nano-silica cage substantially encapsulates the at least one first payload at an encapsulation efficiency of at least 90%,
optionally wherein the nano-silica cage completely encapsulates the at least one first payload,
optionally wherein the at least one first payload is selected from the group consisting of a protein, a peptide, a peptide antigen, an enzyme, an antibody, a nanobody, an antigen binding fragment of an antibody, a single stranded nucleic acid, a double stranded nucleic acid, and a mixture thereof; and
wherein the nano-silica cage and the at least one first payload are both confined within the hollow inner cavity of the YP,
(c) coating the surface of the YP with a mucus penetrating layer, optionally a polydopamine (PDA) layer;
(d) modifying the surface of the YP to display a receptor ligand,
optionally wherein the receptor ligand is an intestinal epithelial cell surface receptor ligand,
optionally wherein the intestinal epithelial cell surface receptor ligand selected from a group consisting of gamma-polyglutamic acid (γ-PGA), wheat germ agglutinin (WGA), concanavalin-A, (ConA), C-terminal Src kinase (CSK) peptide, arginylglycylaspartic acid (RGD) peptide, Fc fragment and a combination thereof;
(e) coating the YP with an enteric coat,
optionally wherein the enteric coat is a polymer,
optionally wherein the enteric coat is a polymer selected from a group consisting of cellulose acetate phthalate (CAP), cellulose acetate trimellate (CAT), hydroxy propyl methyl cellulose phthalate (HPMCP), hydroxyl propyl methyl cellulose acetate succinate (HPMCAS), polyvinyl acetate (PVAP), and methacrylic acid polymers.

24-25. (canceled)

26. The method of claim 23, further comprising the step of loading a coating polymer in the YP, and/or the step of loading a second payload in the YP,
optionally wherein the second payload is selected from the group consisting of a protein, a peptide, a peptide antigen, an enzyme, an antibody, an antigen binding fragment of an antibody, a single stranded nucleic acid, a double stranded nucleic acid, and a mixture thereof.

27-32. (canceled)

33. A pharmaceutical composition comprising a gastric resistant, enteric coated nano-silica yeast particle (YP) delivery system comprising:
(i) a YP with a hollow inner cavity and at least one first payload,
wherein the at least one first payload is substantially encapsulated by a nano-silica cage,
optionally wherein the at least one first payload is completely encapsulated by a nano-silica cage,
optionally wherein the at least one first payload is selected from the group consisting of a protein, a peptide, a nucleic acid, and any combinations thereof; and
wherein the nano-silica cage and the at least one first payload are both confined within the hollow inner cavity of the YP;
(ii) the YP surface is modified with a a mucus penetrating layer, optionally a polydopamine (PDA) layer;
(iii) the YP surface is further modified to display a receptor ligand; and
(iv) The YP is coated with an enteric coat.

34-35. (canceled)

36. A method of treating a disease or disorder in a subject, comprising administering the pharmaceutical composition of claim 33 to a subject in need thereof.

37. A vaccine comprising a gastric resistant, enteric coated nano-silica yeast particle (YP) delivery system comprising:
(i) a YP with a hollow inner cavity and at least one first payload,
wherein the at least one first payload is substantially encapsulated by a nano-silica cage,
optionally wherein the at least one first payload is completely encapsulated by a nano-silica cage, and
wherein the nano-silica cage and the at least one first payload are both confined within the hollow inner cavity of the YP;
(ii) the YP surface is modified with a mucus penetrating layer, optionally a polydopamine (PDA) layer; (iii) the YP surface is further modified to display a receptor ligand; and
(iv) The YP is coated with an enteric coat.

38. (canceled)

39. The vaccine of claim 37, wherein the at least one first payload is selected from the group consisting of a protein, a peptide, a glycoprotein, a lipoprotein, a toxoid, a polysaccharide, a nucleic acid and any combination thereof.

40. A method of preventing a disease condition in a subject, comprising administering to the subject the vaccine of claim 37.
